(19) United States
(12) Patent Application Publication
(10) Pub. No.: US 2005/0202005 A1
(43) Pub. Date: Sep. 15, 2005

(54) Uses of inhibitors for the activation of CXCR4 receptor by SDF-1 in treating rheumatoid arthritis

(75) Inventors: Robert J. Winchester, New York, NY (US); Tetsunori Seki, Roosevelt Island, NY (US); Percio Gulko, Riverdale, NY (US)

Correspondence Address: John P. White;Cooper & Dunham LLP, 1185 Avenue of the Americas, New York, NY, 10036, US

(73) Assignee: The Trustees of Columbia University in the City of New York

(21) Appl. No.: 10/984,482

(22) Filed: Nov. 8, 2004

Related U.S. Application Data
(63) Continuation of application No. 09/500,746, filed on Feb. 9, 2000, now abandoned.
Application No. 09/500,746 is a continuation-in-part of application No. PCT/US99/17178, filed on Jul. 29, 1999.
Application No. PCT/US99/17178 is a continuation-in-part of application No. 09/127,651, filed on Jul. 31, 1998, now abandoned.

(51) Int. Cl. A61K 39/395

(52) U.S. Cl. 424/133.1; 424/144.1; 424/141.1

(57) ABSTRACT

This invention provides a method for treating rheumatoid arthritis or other forms of inflammatory arthritis which comprises administering to a subject an amount of an agent effective to inhibit the activation of the CXCR4 receptor by SDF-1. This invention further provides a composition for treating rheumatoid arthritis comprising an effective amount of an agent capable of inhibiting the activation of the CXCR4 receptor by SDF-1 and a pharmaceutically acceptable carrier. This invention also provides a method for determining whether an agent is capable of inhibiting the activation of the CXCR4 receptor by SDF-1 comprising: (a) contacting cells expressing the CXCR4 receptor in the presence of SDF-1 with the agent under conditions permitting activation of the CXCR4 receptor by SDF-1 if the agent is absent; and (b) determining whether the amount of activation of the CXCR4 receptor by SDF-1 is decreased in the presence of the agent relative to the amount of activation in its absence, such a decrease indicating that the agent is capable of inhibiting the activation of the CXCR4 receptor by SDF-1. Finally, this invention provides agents identified by such a method.

This application is a continuation-in-part application of International Application No. PCT/US99/17178, filed Jul. 29, 1999, which claims priority of U.S. Ser. No. 09/127,651, filed Jul. 31, 1998 the contents of which are hereby incorporated by reference into this application.

Throughout this application various references are referred to within parenthesis. Disclosures of these publications in their entireties are hereby incorporated by reference into this application to more fully describe the state of the art to which this invention pertains.

### BACKGROUND OF THE INVENTION

The architecture, cellular composition and state of cellular activation of the synovial membrane in rheumatoid arthritis have been well described (Winchester 1995, Barland 1962) but fundamental questions still remain unanswered regarding the precise molecular nature and biologic significance of these inflammatory changes. The intimal synovial lining layer that is extensively altered in synovitis synovium through hyperplasia and infiltration is formed by the interaction of two distinct cell types: intimal synoviocytes derived from the fibroblastoid lineage and intercalated, hemopoietically-derived, monocytoid lineage cells (Norton 1966, Burmester 1983, Edwards 1997). During histogenesis of the normal joint the lining cell apparently provides both guidance clues and receptor interactions to the specialized synovial monocytoid cells that result in its incorporation into the lining layer (Winchester 1995). Together, the cells comprising the intimal layer carry out a number of functions responsible for the integrity and sustenance of the joint.

The form and function of the intimal synoviocyte apparently distinguishes them from fibroblastoid cells found deeper in the synovium, although relatively little is known about the differences between these members of the fibroblastoid lineage (Morales-Ducret 1992). Several genes have been identified that are selectively expressed in the normal intimal, but not subintimal synoviocytes including vascular cell adhesion molecule 1 (VCAM-1) (Klareskog 1982), uridine diphosphoglucose dehydrogenase (UDPGD) and decay accelerating factor (DAF) (Morales-Ducret 1992). In chronic synovitis immunopathologic studies have shown that the fibroblastoid intimal synoviocytes respond to the events by proliferating and altering their pattern of gene expression to include expression of a variety of molecules that range from MHC class II structures, through cytokines to enzymes that directly participate in the destructive remodelling of joint tissues (Winchester 1995, Trabandt 1990, Firestein 1990, Arend 1990, Koch 1991, Winchester 1981, Werb 1977). In parallel, some of the fibroblasts in subintimal locations similarly express MHC class II and VCAM-1 (Morales-Ducret 1992, Winchester 1981). However, the performance of more analytic studies of synoviocyte cell biology has been constrained because there is no basement membrane that delimits intimal synoviocytes from the subintimal fibroblastoid cells in either normal or inflamed joint tissues, and the purification and separate culture of these two potentially distinct lineages has been difficult, if not impossible.

For many years it has been recognized that long term cultures of fibroblastoid cells obtained from synovial tissue of individuals with rheumatoid arthritis and marked degrees of intimal hyperplasia continue to exhibit several phenotypes that together are characterized by varying degrees of striking ‘stellate’ or ‘dendritic’ morphology, enhanced growth, increased glucose consumption, altered adherence behavior, constitutive overproduction of metalloproteinases and the elaboration of proinflammatory cytokines (Werb 1977, Caster 1977, Bucala 1991, Smith 1971). The distinctive but not entirely uniform phenotype of rheumatoid synoviocytes is not found in similarly cultured synoviocytes obtained from osteoarthritis synovia that lack lining cell hyperplasia and any inflammatory cell infiltration (Smith 1971). The occurrence of this distinctive phenotype has been shown to be characteristic of, but not unique to, cell lines established from rheumatoid arthritis synovia, as it is also demonstrable in cultures initiated from a number of different entities characterized by chronic inflammation, including osteoarthritis synovia with considerable degrees of inflammation (16). These cell lines have been used to gain a series of interesting insights into the biology of joint inflammation (Bucala 1991, Smith 1971, Wynne-Roberts 1972, Anastassiades 1978, Ponteziere 1990, Goddard 1990, Winchester 1993, Kriegsmann 1995), although the origin of the cells in culture is somewhat uncertain and at least at the time of initiation includes hyperplastic intimal synoviocytes, subintimal synoviocytes, other fibroblastoid cells as well as non mesenchymal cells that do not survive after three passages. We and others have postulated that the distinctive changes in synoviocyte phenotype observed in these cell lines mirror certain similar events occurring in the inflamed synovium itself (Castor 1977, Bucala 1991, Ritchlin 1989, Ritchlin 1994, Lisitsyn 1993).

Finding additional genes that may be selectively expressed in the cultured synoviocyte obtained from inflammatory synovitis would likely provide further insight into the origin of the synoviocytes comprising the cultures, the biology of the intimal synoviocyte and the alterations that this cell and other synovial fibroblasts undergo in synovitis. To further this gene discovery process, a general approach was adapted based on the construction of representational difference libraries (Hubank 1994, Sambrook 1989) that had been used to clone the differences between two complex genomes. It involves a cloning procedure with PCR amplification of cDNA to generate simplified representations of the expressed genes followed by a modified subtractive step and subsequent screening to facilitate the gene identification.

By identification of these genes, it is discovered that SDF-1 are expressed on the synoviocytes which can activate the CXCP4 receptors on lymphocytes and monocytes, either causing them to enter the joint and initiate inflammation through a chemokine effect, or activate these cells that have entered the joint to enhance inflammation.

### SUMMARY OF THE INVENTION

This invention provides a method for treating rheumatoid arthritis or other forms of inflammatory arthritis which comprises administering to a subject an amount of an agent effective to inhibit the activation of the CXCR4 receptor by SDF-1.

This invention provides a composition for treating rheumatoid arthritis comprising an effective amount of an agent capable of inhibiting the activation of the CXCR4 receptor by SDF-1 and a pharmaceutically acceptable carrier.

This invention also provides a method for determining whether an agent is capable of inhibiting the activation of a CXCR4 receptor by SDF-1 comprising: (a) contacting cells expressing the CXCR4 receptor in the presence of SDF-1 with the agent under conditions permitting activation of the CXCR4 receptor by SDF-1 if the agent is absent; and (b) determining whether activation of the CXCR4 receptor by SDF-1 is decreased in the presence of the agent relative to the amount of activation in its absence, such a decrease in the amount of activation indicating that the agent is capable of inhibiting the activation of the CXCR4 receptor by SDF-1. Finally, this invention provides agents identified by such a method.

### BRIEF DESCRIPTION OF FIGURES

FIG. 1 Schematic chart for the identification of genes overexpressed in rheumatoid arthritis synoviocytes.

FIG. 2 Comparison of the amino acid sequence of human semaphorin III, IV, V, and mouse semaphorin E with the predicted sequence of human semaphorin VI. Nucleotide sequence of the cDNA fragment of human semaphorin VI was translated into an amino acid sequence, and compared to that of the corresponding region of human semaphorin III, IV, V and mouse semaphorin E. Conserved amino acids are indicated with boxes. One amino acid gap introduced in the human semaphorin III and V to obtain the best alignment was marked by X.

FIG. 3 Comparison of amino acid sequence of the human N-acetylglucosamine-6-sulfatase and predicted amino acid sequence from the C. elegans cosmid. K09C4 and the clone ts99. Nucleotide sequence of the cDNA fragment of the clone ts99 was translated to an amino acid sequence, and the corresponding region of the human N-acetylglucosamine-6-sulfatase and C. elegans cosmid K09C4 were compared. Conserved amino acids are marked with boxes.

FIG. 4 Representative Northern blot analysis of the isolated clones. lug polyA⁺ RNA was used to run on a 1% agarose gel. The probe used are clone ML2122, clone ML2115, lumican, IGFBP5, SDF-1-α, semaphorin VI, collagenase type IV. The first lane of each blot is RNA from cultured rheumatoid arthritis synoviocytes, and the second lane is RNA from cultured osteoarthritis synoviocytes.

FIG. 5 Lineage relationships of cells derived from a mesenchymal fibroblast precursor. These cell give rise to fibroblasts including the intimal and subintimal.

FIG. 6 Receptor-mediated homotypic cell-cell interaction of fibroblast-like intimal synoviocytes with each other and their heterotypic interaction with monocytoid intimal synoviocytes. The polarized state of the intimal cells is indicated by their interaction of one surface on the right with the subintimal connective tissue matrix and on the left with the hyaluronate-rich synovial fluid.

FIG. 7 Fibroblast-like intimal synoviocyte exhibiting stellate or ÒdendriticÓ morphology, like an astrocyte in culture.

FIG. 8 The third interpretation of the basis of the distinctive phenotype of cultured rheumatoid arthritis synoviocytes. This is the lineage model that bases the distinctive phenotype on the fact that the starting point of the culture differs greatly in the proportion of intimal and subintimal synoviocytes in rheumatoid arthritis and osteoarthritis. This model postulates that the intimal and subintimal synoviocytes have different phenotypes based on their differentiation lineages and that the difference in the phenotype of the cultured cells simply reflects the varying starting proportions of the two cell types.

FIG. 9 Northern analysis of lumican, IGFBP5, SDF-1a, semaphorin VI, collagenase type IV and the two novel transcripts of yet unidentified genes ML2122 and ML2115.

FIG. 10 Directed leukocyte egress. Cytokines act in the endothelium and chemokines act in the leukocyte to regulate their efflux from blood vessels.

FIG. 11 Proposed stages in the development of rheumatoid arthritis.

FIG. 12 SDF-1 Sequence

### DETAILED DESCRIPTION OF THE INVENTION

Throughout this application, reference to specific nucleotides are to nucleotides present on the coding strand of the nucleic acid. The following standard abbreviations are used throughout the specification to indicate specific nucleotides:

| | |
|---|---|
| C = cytosine | A = adenosine |
| T = thymidine | G = guanosine |

This invention provides a method for treating an inflammatory arthritis which comprises administering to the subject an agent that binds an SDF-1 protein expressed on synoviocytes so as to inhibit the interaction of the SDF-1 protein with CXCR4 receptors on lymphocytes and monocytes, and thus treat inflammatory arthritis.

This invention provides a method for treating rheumatoid arthritis or other forms of inflammatory arthritis which comprises administering to a subject suffering from such a condition an amount of an agent effective to inhibit the activation of a CXCR4 receptor by SDF-1, particulary the human CXCR4 receptor. Diseases which represent other forms of inflammatory arthritis are known in the art, and include, but are not limited to, psoriatic arthritis and inflammatory osteoarthritis.

In an embodiment of the invention, the activation is blocked by an agent.

In a further embodiment, the agent is an oligopeptide or a polypeptide. In a further embodiment, the agent is an antibody or a portion of an antibody wherein the antibody is preferably human, chimeric, or a humanized antibody or portion thereof.

In another embodiment, the agent is a nonpeptidyl agent. In a further embodiment, the nonpeptidyl agent is a bicyclam such as AMD3100 (Donzella, G. A., et al (1998), AMD3100, a small molecule inhibitor of HIV-1 entry via the CXCR4 co-receptor, Nature Medicine, 4:72-77). AMD3100 is a bicyclam derivative and is representative of this class of chemicals. See DeVreese, K. et al., Antiviral Research, 29, 209-219 (1996).

This invention provides a composition for treating rheumatoid arthritis comprising an effective amounts of an agent capable of inhibiting the activation of the CXCR4 receptor by SDF-1 and a pharmaceutically acceptable carrier. In an embodiment, the agent is oligopeptide. In another embodimet, the agent is a polypeptide. In a further embodiment, the agent is an antibody or a portion of an antibody, wherein the antibody is, a human chimeric or humanized antibody.

Pharmaceutically acceptable carriers are well-known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

The agent may be administered orally, parenterally or intra-articularly.

In another embodiment, the agent is a nonpeptidyl agent. In an embodiment, wherein the nonpeptidyl agent is a bicyclam such as AMD3100.

This invention also provides a method for determining whether an agent is capable of inhibiting the activation of the CXCR4 receptor by SDF-1 comprising: (a) contacting the cells expressing CXCR4 receptor in the presence of SDF-1 with the agent under condition permitting activation of the CXCR4 by SDF-1 if the agent is absent; and (b) determinig whether the amount of activation of the CXCR4 receptor by SDF-1 is decreased in the presence of the agent relative to the amount of activators in its absence, such a decrease in the amount of activation indicating that the agent is capable of inhibiting the activation of the CXCR4 receptor by SDF-1. In an embodiment, the CXCR4 is expressed in cells. In a further embodiment the cells are lymphocytes or monocytes. In a separate embodiment the CXCR4 expressed artificially in prokaryotic or eukaryotic cells. Such cells include but are not limited to bacterial, fungal, plant or animal cells using methods well known in the art.

Finally, this invention provides an agent identified by the above-described method and a composition comprising an amount of an agent identified by the above-described method effective to inhibit the activation of the CXCR4 receptor by SDF-1 and a suitable carrier. In the practice of this invention the agent may be, but is not limited to a polypepteide of VCAM-1, a 110 kd member of the immunoglobulin gene superfamily, and Mac-2 binding protein (Mac-2BP), also termed 90 k tumor associated protein or IGFBP5 (insulin-like growth factor binding protein-5).

As used herein “agent” means an antibody, polypeptide, peptide, analogue of a peptide, a nucleic acid, or an organic molecule that is capable of binding an SDF-1 protein expressed on synoviocytes so as to inhibit the interaction of the SDF-1 protein with CXCR4 receptors. In the case of polypeptides, the polypeptide may be an advanced glycation endproduct polypeptide or a portion thereof. The polypeptide may be synthesized chemically or produced by standard recombinant DNA methods. In the case of antibodies the antibody may be capable of specifically binding to the SDF-1 receptor. In another case the antibodies may be capable of specifically binding to the CXCR-4 reseptor. The antibody may be a monoclonal antibody, a polyclonal antibody. The portion or fragment or a F_{c} fragment. The portion or fragment of the antibody may comprise a complementarity determining region or a variable region.

The present invention also provides for a method for inhibiting. CXCR-4 interaction with a protein receptor of a protein associated with the genes identified in rheumatoid arthritis when the receptor is on the surface of a cell, which comprises contacting the cell with an amount of an inhibitor of said interaction effective to inhibit interaction of the CXCR-4 with the protein receptor.

The present invention provides for an isolated peptide having an amino acid sequence of SDF-1, SDF-1α or SDF-1β corresponding to the amino acid sequence of the II56 kd protein. 8+34, IGFBP5, 30+77 and SDF-1α 28+41. The present invention also provides for a method of treating or ameliorating symptoms in a subject which is associated with a disease, wherein the disease in an inflammatory disease of the joint such as rheumatoid arthritis, which comprises administering to the subject an amount of the isolated peptide of the present invention or an agent capable of inhibitiing the interaction between CXCR-4 and SDF-1, effective to inhibit the interaction so as to treat or ameliorate the disease or condition in the subject. The method may also prevent such conditions from occurring in the subject.

The present invention provides for an isolated peptide having an amino acid sequence which corresponds to the amino acid sequence of 36 of the amino acids of SDF-1 protein.

In the practice of the method the route of administration and frequency of administration is subject to various variables such as age and condition of the subject, area of the subject to which administration is desired and the like.

In connection with the method of this invention, a therapeutically effective amount may include dosages which take into account the size and weight of the subject, the age of the subject, the severity of the symptom, and the efficacy of the agent. One of ordinary skill in the art would be readily able to determine the exact dosages and exact times of administration based upon such factors. For example, a therapeutically effective amount may be a dose of from about 0.1-10 mg/kg. In this regard, the dose may also be administered as a single dose or as a series of doses over a period of time.

The use of antibodies, polypeptides, peptides or analogues of peptides to treat rheumatoid arthritis is known in the art. The following publications are hereby incorporated by reference: Rankin, E. C., et al. (1995) “The Therapeutic Effects of Engineered Human Anti-Tumour Necrosi Factor α antibody (CDP571) in Rheumatoid Arthritis”; Dinant, H. J. and Dijkmans, B. A. (1999) “New Therapeutic. Targets for Rheumatoid Arthritis”; and Maini, R. N. et al. (1998). “Therapeutic Efficacy of Multiple Intravenous Infusions of Anti-Tumour Necrosis Factor Alpha Monoclonal Antibody Combined with Low-dose Weekly Methotrexate in Rheumatoid Arthritis”. Discolosed is the use of engineered human antibody that neutralizes tumour necrosis factor alpha was administered intravenously in single doses of 0.1, 1.0 or 10 mg/kg to patients with active rheumatoid arthritis. Short-term significant beneficial effect on Rheumatoid Arthritis disease activity has been established in a small but rapidly growing number of double-blind placebo-controlled trials now including recombinant human IL-1 receptor antagonist, chimeric (mouse/human) monoclonal antibodies and recombinant human tumour necrosis factor receptor fusion protein. The disclosures of the publications referred to herein, in their entireties, are hereby incorporated by reference into this application in order to more fully describe the state of the art as known to those skilled therein.

In an embodiment of this invention an improvement in arthritis is associated with anti-chemokine therapy by blocking overexpression of SDF-1 in normal synoviocytes using antibody theraphy specific to SDF-1.

In an embodiment of this invention a soluable CXCR-4 biological agent recombinant receptor antagonist to SDF-1 may be used as an antagonist to SDF-1 to bind circulating SDF-1 thereby preventing SDF-1 from binding the CXCR-4 receptor. In an embodiment of the invention the soluable CXCR-4 binds to a receptor on a monocyte, an example of which. Is IGFBP5.

In another embodiment small molecules may be used to block signal transduction of SDF-1.

This invention will be better understood from the Experimental Details which follow. However, one skilled in the art will readily appreciate that the specific methods and results discussed are merely illustrative of the invention as described more fully in the claims which follow thereafter.

Experimental Details

First Series of Experiments

Synoviocyte culture. Synovial tissue was obtained at the time of joint replacement from a classic rheumatoid arthritis with 10-12 layers of hyperplastic lining cells which intensively expressed HLA-DR and HLA-DQ molecules, and showed replacement of the superficial lining layer with monocytoid cells and an extensive subintimal infiltration of lymphocyte aggregates and monocytes. The osteoarthritis sample was taken from a synovium that had no lining cell hyperplasia and no subintimal cellular infiltration. The tissue was minced, enzymatically dissociated and cultured through five passages in Isocove's Modified Dulbecco's Media (Gibco, Grand Island, N.Y.) supplemented with selected lots of 10% fetal calf serum (Gibco, Grand Island, N.Y.) and 1% penicillin-streptomycin (Sigma, St. Louis, Mo.) as described (Edwards 1997). The resulting cells which presumably included intimal and subintimal synoviocytes in varying proportions according to their proportion in the starting material were grown to confluence and passaged by brief exposure to dilutions of 1% trypsin-EDTA (Sigma, St. Louis, Mo.).

Construction of the subtraction library and preliminary sequencing. PolyA+ RNA was isolated from the fifth passage synoviocytes using a mRNA Isolation Kit (Stratagene). 2 ug of twice purified polyA+ RNA was used as a template for cDNA synthesis in the RiboClone cDNA Synthesis System (Promega). The synthesized cDNA was ligated with the oligonucleotides GATCCGCGGCCGC and GCGGCCGCGT as described (Hubank 1994). After selection of fragments larger than 250 nucleotides by fractionation through a Sephacryl S-400 column (Pharmacia) and phosphorylation with T4 polynucleotide kinase, the cDNA was digested with the restriction enzyme MboI. The fragments were then ligated with oligonucleotides J-Bam-24 ACCGACGTCGACTATCCATGAACG and J-Bam-12 GATCCGTTCATG, and amplified as described (Hubank 1994). The PCR products, after fractionation through Sephacryl S-400 column, were digested with MboI and they comprised the primary amplicon. DNA from rheumatoid arthritis synoviocytes was further ligated with oligonucleotides N-Bam-24 AGGCAACTGTGCTATCCGAGGGAG and N-Bam-12 GATCCTCCCTCG. The hybridization was performed as described (26) except that the ratio of tester and driver was kept 1:100 throughout. 10 ug of the osteoarthritis primary amplicon were hybridized with 0.1 ug of the rheumatoid arthritis primary amplicon in 5 ul of 24 mM EPPS, pH8.0, 1 mM EDTA, 1M NaCl for 20 hr at 67 C. The hybridized DNA was subjected to 10 cycles of PCR with N-Bam-24 as a primer, followed by digestion with mung bean nuclease. One four hundredth of the digests was further amplified for 20 cycles. After digestion with MboI, the DNA product was ligated with oligonucleotides R-Bam-24 AGCACTCTCCAGCCTCTCACCGAG and R-Bam-12 GATCCTCGGTGA. Hybridization and amplification steps were repeated. After redigestion with MboI, the resulting differentially subtracted cDNA fragments were cloned into a BamHI site of the plasmid pUC18. The recombinants were inoculated in an ordered grid pattern on nitrocellulose filters, which were then probed with the osteoarthritis cDNA amplicon ³²P-labeled with the Megaprime DNA labeling System (Amersham). The DNA sequence of the non-hybridized recombinants was determined in an Applied Biosystems DNA Sequencer Model 3.73A or 377 using standard dye terminator chemistry. The seqman module of the Lasergene program (DNAstar) was used for identification of homologous recombinants and grouping them into groups. The Genman module of the Lasergene program was used to search the GenBank databases including the expressed sequence tag database on. CDROM. BLAST was used to verify the identification of sequences that showed no homology with entries in the CDROM database.

Northern blot analysis. Probes were prepared from the clones by PCR amplification of the inserts, digestion with MboI and isolation by electrophoresis on a 1% agarose gel. lug of the once purified polyA+ RNA of the same preparation used for the construction of subtraction library was run on a 1% agarose gel, containing 1.9% formaldehyde and hybridized with the ³²P-labelled probes as described (Seki 1989). The membranes were re-probed several times after stripping off the previous probe.

Construction of a rheumatoid arthritis cDNA library. The same preparation of the cDNA from the rheumatoid arthritis patient used for the construction of the subtraction library was ligated with EcoRI adapters. These constructs were cloned into λgt10 by standard procedures and the library was screened as described previously (Shirozu 1995).

Experimental Results

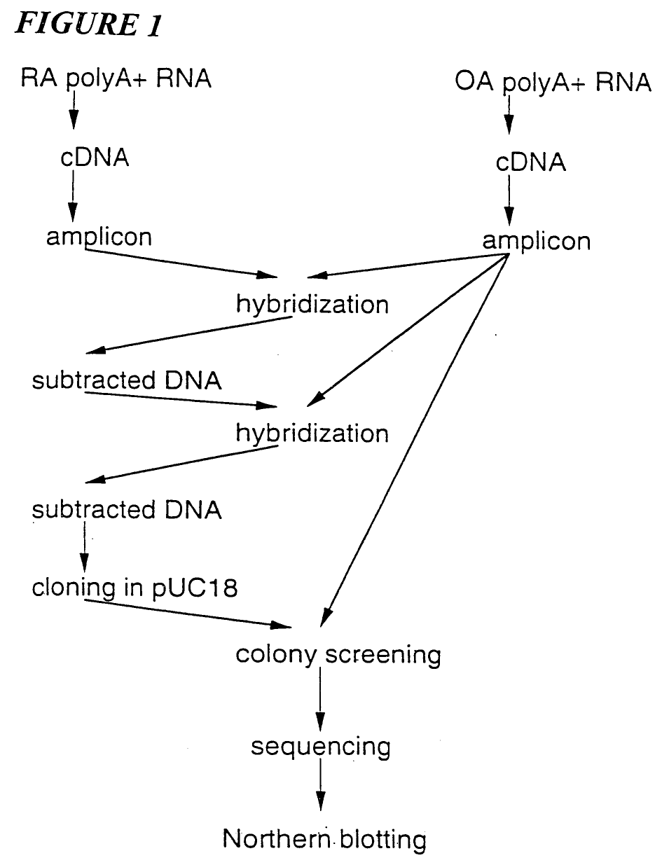

Identification of Genes Differently Represented in the Cultured Rheumatoid Arthritis and Osteoarthritis Synoviocytes. To identify genes that may be differently expressed in the cultured rheumatoid arthritis and osteoarthritis synoviocytes, cell lines originating from a carefully selected highly inflammatory rheumatoid arthritis synovium and an osteoarthritis synovium with no lining cell hyperplasia or inflammatory cell infiltration were chosen. Two subtraction cycles were performed between polyA+ RNA from fifth passage rheumatoid arthritis and osteoarthritis synoviocytes followed by negative screening of the resulting difference representation clones with a probe consisting of the ³²P-labelled osteoarthritis synovial fibroblastoid cDNA amplicons (FIG. 1). 319 recombinant clones were selected for further analysis by DNA sequencing.

Nucleotide sequencing revealed that many of those 319 recombinants had the same sequence, comprising of distinct 24 sequence groups. As would be expected, the number of recombinants representing each group varied considerably, ranging from just one to as many as 77 recombinants (Table 1). Comparison of the sequence with the GenBank database revealed that 16 sequence groups showed more than 97% homology with the previously identified human genes (Table 1). In the case of insulin-like growth factor binding protein 5 (IGFBP5) and interferon-inducible 56 kd protein (II56kd protein) two cDNA fragments derived from the different portion of the same gene.

TABLE 1
List of the identified genes and number of obtained
clones.

| Name of gene | Number of Clones |
|---|---|
| Group 1* | |
| Manganese superoxide dismutase | 8 |
| Collagenase type IV | 4 |
| Complement factor B | 4 |
| α-B crystallin | 1 |
| Interferon-gamma IEF SSP 5111 | 1 |
| B94 protein | 1 |
| HLA-E heavy chain | 1 |
| NMB protein | 9 |
| Muscle fatty-acid-binding protein | 1 |
| Group 2* | |
| VCAM-1 | 2 |
| II56 kdprotein | 42 |
| 71 kd 2′-5′-oligoadenylate | 1 |
| synthetase | |
| Mac2 binding protein | 21 |
| Biglycan | 16 |
| Lumican | 3 |
| IGFBP5 | 107 |
| SDF-1-α | 69 |
| Semaphorin VI | 1 |

On Northern blot analysis, Group 1 genes showed little difference in the intensity of hybridization between cultured rheumatoid and osteoarthritis synoviocyte RNA. Group 2 genes exhibited overexpression in rheumatoid arthritis synoviocytes compared with osteoarthritis synoviocyte. In the case of the genes that were represented in two different sequence groups, a total number of clones are shown in the table. Those genes are II56 kd protein, 8+34, IGFBP5, 30+77, and SDF-1α 28+41.

Characterization of novel genes. Of the remaining 8 sequence groups, two highly represented clones with copy numbers of 28 and 41 in the library had 32% and 25% similarity, respectively, to the 3′-untranslated region of the mouse SDF-1α. These fragments hybridized with the same clones from the λgt10 rheumatoid arthritis synoviocyte library, indicating that they derived from the same transcript.

The nucleotide sequence of the clones showed high homology with mouse SDF-1α in the coding region (data not shown), and was almost identical with the subsequently published sequence of the human SDF-1α gene (Tan 1990).

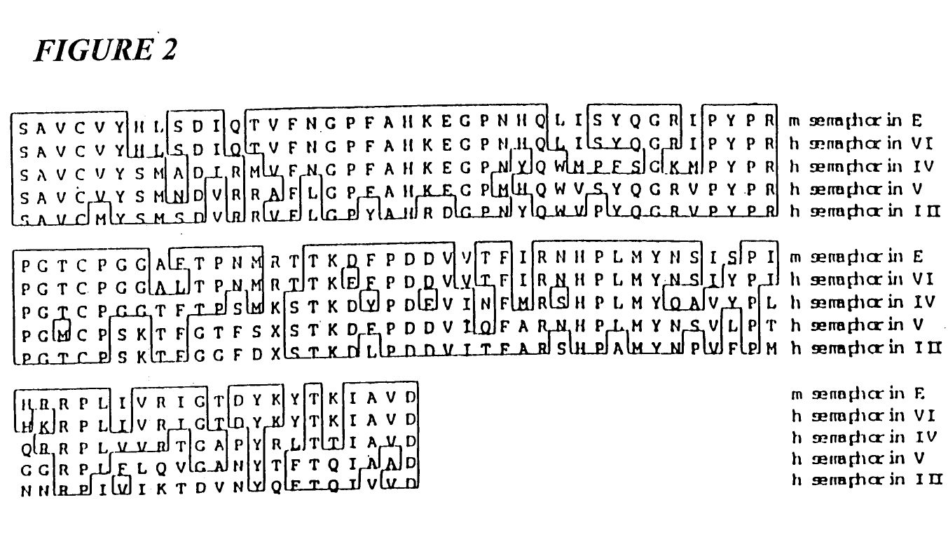

Another clone was found to have 90% homology with mouse semaphorin E at the nucleotide level and 94% at the putative amino acid level. This suggested that the isolated clone was a human homologue of the mouse semaphorin E, and it was tentatively named human “semaphorin VI”. A comparison of the amino acid sequences with the previously described human semaphorins III, IV, V and mouse semaphorin E is shown in FIG. 2.

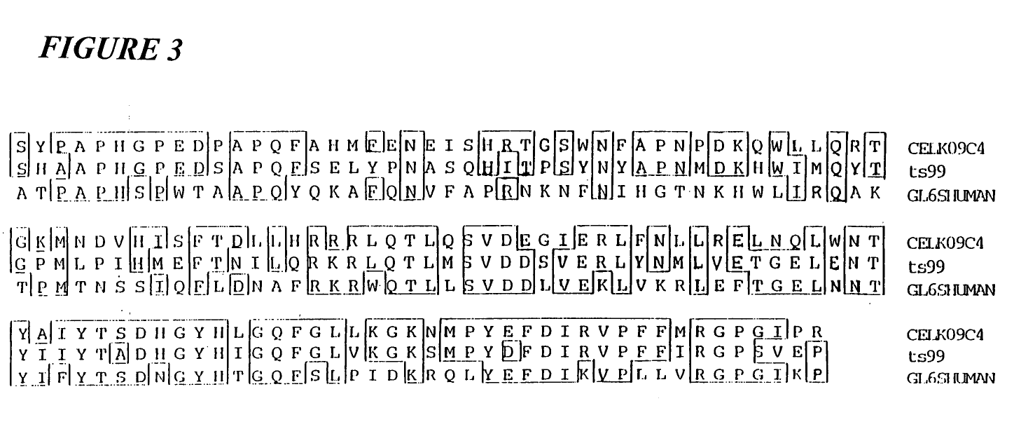

Analysis of another clone showed some homology at the nucleotide level and more significantly at the putative amino acid sequence level with a variety of sulfatases. Among human genes the greatest similarity was with the human N-acetyl-glycosamine sulfatase. However the sequence of this clone was most homologous with the putative amino acid sequence derived from the C. elegans genomic cosmid KO9C (FIG. 3).

A portion of the sequence of clone ML2115 was 99% identical with the EST sequence AA447232. The remaining clones did not show significant homology to any known genes in either nucleotide level nor in translated amino acid level, and their identification is continuing.

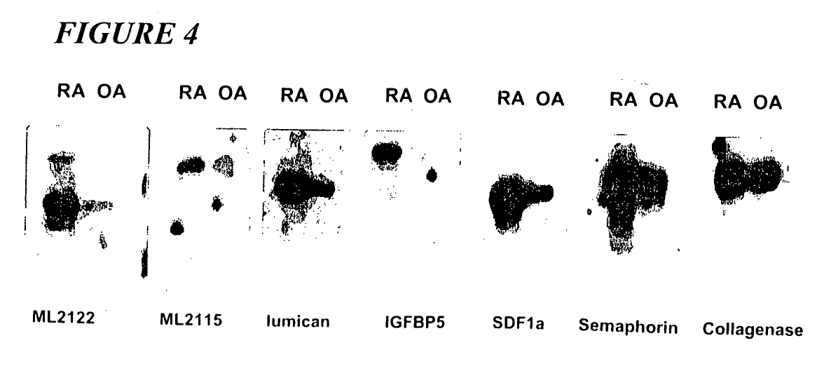

Northern analysis. To determine the actual difference in level of expression of the genes characterized by the 24 different recombinant clones, Northern analysis of polyA+ RNA from the two cell lines used to make the difference library was performed. The level of GAPDH expression was not detectably different between both synoviocytes (data not shown). FIG. 4 illustrates a representative gel using inserts of clones as probes from, lumican, IGFBP5, SDF-1α, semaphorin VI, collagenase type IV and the two clones, ML2122 and ML2115 which did not show appreciative homology to the known genes As shown, the expression of collagenase-type IV did not differ significantly between the two-RNA preparation. Similarly, the expression of genes depicted in Group 1, Table 1, such as HLA-E, α-B-crystallin and manganese superoxide dismutase had minimally increased or essentially equivalent levels of expression in the osteoarthritis and rheumatoid arthritis synoviocyte cell lines.

However, of the genes identified in this study, 11 had moderate to marked differentially elevated expression in the rheumatoid arthritis synoviocyte line used for the subtraction (Table 1. Group 2), suggesting that these genes were constitutively overexpressed in cultured rheumatoid arthritis synoviocytes. These 11 genes included: VCAM-1, Mac-2 binding protein (Mac-2BP), IGFBP5, biglycan, lumican, SDF-1α, II56 kd protein, 71 kd 2′-5′ oligoadenylate synthetase, semaphorin VI, and two clones ML2115 and ML2122. The clone ML2115 hybridized with a 6 kb mRNA. The clone ML2122 hybridized with three species of mRNA of which 4.7 kb was the major one (FIG. 4). The characterization of these clones is being continued.

Since SDF-1α has an alternatively spliced form SDF-1-β with which it shares the most of coding region but a different 3′-untranslated region (30), the expression of SDF-1β was investigated. Its expression was also found to be increased in parallel with that of SDF-1α in the rheumatoid arthritis synoviocytes compared to the osteoarthritis cells (data not shown).

Experimental Discussion

The objective of the present study was to develop a method to identify additional genes that comprise the distinctive biochemical and cell physiologic phenotype of cultured rheumatoid arthritis fibroblastoid synoviocytes. Of 24 genes characterized by this procedure, 11 were found to be constitutively overexpressed by Northern analysis in the rheumatoid arthritis synoviocyte culture used for subtraction and three were novel genes. The relatively unbiased gene discovery approach used to subtract differential representations of the expressed genes in the two prototype cell lines is a general method useful for the identification of differentially expressed genes. The characteristics of the genes identified in the present study direct increased attention to the possibilities that synoviocytes from synovia with marked lining cell hyperplasia are characterized both by different matrix and cell-cell interactions and by the fact that they likely provide guidance clues and sites for receptor interaction to infiltrating monocytes and lymphocytes during normal histogenesis of the synovial lining, providing a mechanism for the location of monocyte lineage cells in the intimal layer. Moreover, in an exaggerated mode of leukocyte ingress that could occur during synovial hyperplasia, these gene products might foster the localization of an immune or autoimmune response to the joint. Taken together the results direct further attention to the role of mesenchymal cells in immune-mediated diseases.

In the present experiments special attention was directed to the selection of the tissue source of the two cell lines used in the subtraction. Prior studies showed that cell lines obtained from patients clinically characterized as osteoarthritis with various degrees of inflammatory synovitis elaborated proinflammatory cytokines in patterns often similar to those found in rheumatoid arthritis samples (Smith 1971, Ritchlin 1994). In this study the reference synovial sample was from a patient with osteoarthritis who had no evidence of synovitis with only a single cell layer of intimal synoviocytes. In contrast the rheumatoid arthritis synovium used for gene isolation had 10-12 layers of hyperplastic lining cells. It should be stressed that a limitation of this study is that it is not possible to identify the site of origin in the synovial lining of the cultured synoviocytes, although application of reagents directed to identification of these products of these genes in situ should facilitate resolving the question of their origin.

The gene discovery approach used in this work was initially developed to detect the absolute difference between two genomes where each gene is present in the same ratio (Hubank 1994). Because of the differences in the number of each mRNA species and the likelihood that the frequencies of certain mRNA species relatively differed between cultured rheumatoid arthritis and osteoarthritis synoviocytes, the subtraction steps were modified by reducing the ratio of the tester and driver DNA. This had the effect of decreasing the completeness of the subtraction step, but increasing the possibility of discovering genes expressed at a variety of different levels in the two cell lines. To compensate for any potential inefficiency of subtraction, a negative selection screening step was added using the driver osteoarthritis synoviocyte cDNA amplicon as a probe, and the constitutive increase in expression of the identified genes was confirmed in Northern analysis.

Several technical points require comment. The cDNA synthesis was primed with oligo (dT) to bias the ultimate library towards one rich in 3′-untranslated regions, because the nucleotide sequence of this region is more divergent than that of the coding regions. The restriction enzyme MboI was chosen to create DNA fragments of relatively small size to facilitate efficient and even amplification by PCR, and to increases the chance of isolating genes which are differentially spliced and/or members of a supergene family. The DNA fragments were fractionated through a Sephacryl S-400 column to avoid biased amplification of numerous fragments smaller than 250 nucleotides.

The subtractive method is less influenced by differences in a low copy number mRNA species than the related differential display method, however the number of recombinants analyzed places a sampling error limit on the identification of a rare species. In the present study, some differentially expressed genes were identified only by the presence of a single recombinant. There are additional technical reasons, such as the absence of appropriate Mbo 1 sites why some genes previously expressed in cultured inflammatory synoviocytes might not be identified (Smith 1971, Lisitsyn 1993, Koths 1993).

Of the 11 genes constitutively increased in expression in the rheumatoid synoviocytes, VCAM-1, a 110 kd member of the immunoglobulin gene superfamily, and Mac-2BP, also termed ‘90 k tumor associated protein’, both exhibit properties that suggest they could mediate heterotypic binding of monocyte-lineage intimal synoviocytes to fibroblastoid lineage synoviocytes. VCAM-1 has been previously described as markedly increased on rheumatoid arthritis synoviocytes (Winchester 1995, Ritchlin 1989) and the identification of VCAM-1 by this difference method supports the validity of this gene discovery approach for intimal synoviocytes. VCAM-1 binds circulating monocytes and lymphocytes expressing the α₄β₁ (VLA4) integrin. Mac-2BP, a heavily N-glycosylated secreted protein which binds stoichiometrically to the macrophage-associated lectin Mac-2 (galectin-3)(Inohara 1996, Yu 1995), also binds to the monocyte CD14 structure in the presence of LPS and LBP (34). Binding of Mac-2BP to these receptors initiates monocyte-lineage cells to secrete IL-1 and IL-6 and increases their expression of ICAM-1 (Ullrich 1994, Luo 1995). This alteration in monocyte state could be one of the factors modulating the cell into a synovial lining macrophage.

The overexpression of the semaphorin VI by synoviocytes is intriguing because the semaphorins are a family of transmembrane signalling and secreted guidance glycoprotein molecules that are implicated in directing axonal extension (Hall 1996). However, in view of the relatively small number of axons in the synovium, it seems unlikely that the physiologic role of the semaphorin VI molecule is to signal through an axonal receptor. Rather, one might conjecture semaphorin VI plays some role in chemotaxis of monocytes and their differentiation. Suggesting a broader role of semaphorin molecules in cellular interaction, CD100 which plays a role in B-cell activation parallel to that of CD40 ligand has recently been identified as a member of this family (Mangasser-Stephan 1997). A report of the overexpression of semaphorin VI gene in rheumatoid arthritis synovial fibroblastoid cells by the differential display method appeared while this manuscript was in preparation (Nagasawa 1994).

Another molecule constitutively expressed by the rheumatoid synoviocyte was the chemokine SDF-1α. It was first identified as a pre-B cell growth stimulating factor produced by marrow stromal cells (Tashiro 1993, D'Apuzzo 1997). SDF-1-α attracts pro- and pre-B cells (Ajuti 1997) as well as CD34+ hematopoietic progenitor cells (Nagasawa 1996). Mice genetically deficient for SDF-1α lack B-cells and have hematopoiesis only in their liver (44). SDF-1α is the ligand for the CXCR-4 chemokine receptor that serves as a co receptor for entry of T-tropic syncytial inducing forms of HIV into T-cells (Bleul 1996). SDF-1α has recently been the subject of an interesting series of studies that demonstrated this chemokine to be a highly efficacious transendothelial chemoattractant for both monocytes and T-lymphocytes (Rada 1993). It is not clear that SDF-1β has a biologic activity different from that of SDF-1α at the moment. We speculate that the production of SDF-1 by intimal synoviocytes in the normal joint could act as a guidance cue for the continual entrance into the intimal synovial membrane of monocyte lineage precursors committed to differentiation into phagocytic lining cells. Similarly SDF-1 and other chemokines elaborated by the normal synoviocytes may act to enhance the ingress of lymphocytes into the joint tissues to facilitate physiologic surveillance functions.

Several genes were identified as constitutively expressed, indicating the possibility of altered cell-matrix interactions as part of the distinctive rheumatoid arthritis synoviocyte phenotype. Lumican is a keratan sulfate proteoglycan that plays a critical role in the basis of corneal transparency (Grover 1995). In adult cartilage lumican exists predominantly in a glycoprotein form lacking keratan sulfate (Funderburgh 1997). Macrophages do not adhere to intact corneal keratan sulfate proteoglycans but attach and spread rapidly on the lumican core protein after the removal of keratan sulfate chains (Hildebrand 1994). This observation suggests some species of lumican could also act to localize macrophages to sites of the synovium. Biglycan, a dermatan sulfate-proteoglycan, is both induced by TGF-β, and binds TGF-β β (Ungefroren 1996), suggesting that biglycan may down regulate TGF-β activity by sequestering this growth factor in the extracellular matrix. IL-6 stimulates the expression of biglycan, while TNF-α depresses its expression (Jones 1993). IGFBP5, was the most highly represented species in the difference library. This molecule increases IGF-1 binding to the fibroblast membrane by attaching to the extracellular-matrix proteins, types III and IV collagen, laminin and fibronectin (Tyler 1989). IGFBP5 may have an antiinflammatory role that opposes the effect exhibited by IL-1 and TNF-α of stimulating proteoglycan degradation and decreasing proteoglycan synthesis (Pash 1996). The observation that IGFBP5 is further induced by exposure of cells to prostaglandin E2 (54) is of interest with respect to the pattern of morphologic change and gene activation observed in synoviocyte cultures upon addition of this agent (Marie 1992).

The 71 kd 2′-5′ oligoadenylate synthetase is a subunit of one of several interferon-induced enzymes that, when activated by double-stranded RNA, convert ATP into 2′-5′ linked oligomers of adenosine (Wathelet 1986). The interferon-inducible 56 kd protein is of unknown function, but in common with 2′-5′ oligoadenylate synthetase is strongly induced by interferons (Mellors 1961). The expression of these two genes directs attention to the presence of activation-like features in the phenotype of the rheumatoid arthritis synoviocytes.

In prior studies it was found that the relative overexpression of known genes comprising the distinctive phenotype of cultured inflammatory synoviocytes varied somewhat from cell line to cell line (Smith 1971, Lisitsyn 1993). Preliminary evidence using these newly isolated genes indicates similar sample to sample variation in the relative degree of expression of one overexpressed gene relative to another by Northern analysis. Similarly, additional studies will be required to determine whether the levels of expression of the remaining genes that were not preferentially overexpressed in rheumatoid synoviocytes distinguish synoviocytes in general from fibroblastoid cells in other anatomic sites.

The identification of a group of constitutively overexpressed genes in this study is relevant to the three principal cell biologic possibilities explaining the origin of the distinctive phenotype of these cultured rheumatoid synoviocytes. We and others have postulated that the phenotype could result from sustained modulation of gene expression in several fibroblast lineage cells of the joint that developed as a response to prolonged paracrine signalling through products of a local immune response, analogous to a phenotypic imprinting process (Barland 1962). A second possibility is that the cells are primarily ‘transformed’ as suggested by Gay and colleagues (Firestein 1990). However, perhaps most likely in view of the features of the genes isolated in this study, is a third possibility that the phenotype exhibited by these cells is similar to that of the normal intimal synoviocyte. Thus at the start of an experiment, a culture derived from rheumatoid arthritis synovia characterized by marked intimal synoviocyte hyperplasia would contain an increased proportion of intimal lining synoviocytes that are responsible for the resulting phenotype of the cultured cells because of their lineage difference in patterns of gene expression.

Each of these three potential origins shares in common the possibility that the presence of increased quantities of these guidance and cell interaction molecules may itself create a novel synovial microenvironment that could facilitate interactions with monocyte lineage cells and foster the entry of large numbers of inflammatory and immune leukocytes. The first two mechanisms imply that the contribution of synoviocytes to the cell biologic basis of synovitis is qualitatively based due to the presence of abnormally activated or modulated cells while the third mechanism implies a quantitative over representation of members of a normal cell lineage that physiologically exhibits distinctive properties . . . In each case, the resulting environment may modulate or deviate an ongoing immune response and reenforce its subsequent evolution into an autoimmune process.

Since inflammatory imprinting or hyperplasia could be initiated by a non specific minor traumatic event or even driven by a local immune response to a common pathogen, this might provide a non antigen-specific mechanism for localizing potential pathogenic immune responses to the joint. For example, an additional action of SDF-1 at higher concentrations could be the facilitation of earlier stages of peripheral B-cell development in the synovial milieu that are relevant to the presence and maturation of abundant B-cells in the rheumatoid synovium and to their production of rheumatoid factors (Oritani 1996). Furthermore, several additional molecules produced by the synoviocyte can interact to facilitate other aspects of B-cell development. IL-6, a cytokine with effects on B-cell differentiation, is constitutively increased in synoviocytes obtained from rheumatoid arthritis patients (Smith 1971) and its synthesis by monocytes is induced by Mac-2BP, as described above. Interleukin 7-dependent proliferation of pre-B cells is also enhanced upon exposure to biglycan (Grimley 1966). Similarly these molecules could attract and facilitate interaction with and activation of monocytes. For example, Mac-2BP which induces homotypic monocyte aggregation and activation (Yu 1995) could be a factor present in supernatants from cultured rheumatoid arthritis synoviocytes that induces blood monocytes to form giant cells (Grimley 1966). Thus, along with the variety of genes that mediate the well recognized effector functions of matrix remodelling and tissue destruction (Marie 1992), the genes expressed by the mesenchymal cells of the joint may affect antigen non specific immune localization or amplification mechanisms that could play a role in the puzzling phenomenon of why localized joint inflammation develops in many disparate diseases in the setting of immune responses that apparently have little to do with the joint.

Second Series of Experiments

Immunolocalization of SDF-1 and CXCR-4 to Different Cells in the Joints of Patients with Rheumatoid Arthritis.

Objective: In support of the prior observation of the synthesis of SDF-1 on Northern analysis by cultured synovial lining cells from rheumatoid arthritis and other forms of inflammatory arthritis, the synovial tissues of patients with rheumatoid arthritis were studies using a polyclonal goat anti SDF-1 antibody. Similarly, the tissue was studied for the expression of CXCR4, the receptor for SDF-1.

Results: The hyperplastic layer of fibroblastoid synovial lining cells showed intense staining for the presence of SDF-
1. The lymphocytes and monocytes infiltrating in the sub lining cell region of the joint exhibited intense staining for the expression of CXCR4. Similarly, the monocyte-lineage cell in the synovial lining, but not the fibrolastoid synovial lining cells also expressed CXCR4.

Interpretation: The observations are consistence with the first series of experiments. That SDF-1 is made by fibroblastoid synovial lining cells and that this chemokine attracts lymphocytes and monocytes into the joint tissue to cause join inflammation.

Third Series of Experiments

Expression of Chemokine SDF-1 by Intimal Synoviocytes. The chemokine stromal derived factor-1 (SDF-1) was first identified as a pre-B cell growth stimulating factor produced by marrow stromal cells necessary for its population by pro- and pre-B cells and CD34+ hematopoietic progenitor cells. SDF-1 has known to be highly efficacious transendothelial chemoattractant for monocytes and T-cells. The SDF-1 receptor, CxCR4, also serves as a co-receptor for HIV entry into T cells. SDF-1 was identified as a gene overexpressed by cultrured synovial fibroblastoid cells from an individual with rheumatoid arthritis (RA) compared with those from osteoarthritis (OA) by differential subtraction. To investigate whether SDF-1 is generally overexpressed in RA synovial fibroblastoid cell lines, Northern analysis was performed with RNA from fibroblastoid cell lines of 11 RA and 2 OA samples. Eight of the RA lines were from synova with marked lining cell hyperplasia, massive inflammatory infiltration and neovasculization. All 8 exhibited moderate to marked overexpression of SDF-1. The remaining 3 RA individuals had only mild infiltration with little lining cell hyperplasia but considerable neovasculization. These 3 RA and 2 noninflammatory OA cell lines had much lower expression of SDF-1, suggesting a correlation between the level of SDF-1 expression in synoviocyte lines and features of the tissue from which they were derived. Staining of synovial tissues from 3 OA and 2 RA synovia with a polyclonal antibody to SDF-1 revealed 60-70% positivity of intimal synoviocytes in OA. In RA there was markedly stronger and more extensive SDF-1 staining in the hyperplasic lining with additional staining of some subintimal fibroblastoid cells. The results suggest that increased SDF-1 elaboration by intimal synoviocytes and possible other fibroblastoid cells may participate in the pathology of RA by enhancing recruitment of monocytes and T-lymphocytes into the synovium.

Fourth Series of Experiments

The joint is a functionally unique structure primarily formed from mesenchymal cells. Its distinctive cavity is mainly lined by specialized cells belonging to the fibroblast lineage that are designated as fibroblast-like intimal synoviocytes. Through their unique and increasingly defined pattern of gene expression the fibroblast-like intimal synoviocytes appear to be differentiated to perform a series of functions critical to the biologic function of the normal joint. The characteristics of the fibroblast-like intimal synoviocyte and its pattern of gene expression suggests that this cell is closer to the undifferentiated mesenchymal stem cell than to the typical well-differentiated fibroblast. This phenotype also appears to confer the potential for a special role in fostering the development and synovial localization of the autoimmune response underlying rheumatoid arthritis, and to participate in joint destruction.

In addition to the fibroblast-like intimal synoviocytes [1, 2] that account for approximately two thirds of the lining cells in an uninflammed joint, the intima contains a second intercalated cell type determined by their morphology, phenotype and function to be derived from the CD14 positive branch of the monocyte lineage [3]. This latter cell is designated the monocytoid intimal synoviocyte. These two types of lining cells were originally named “type B” and “type A” respectively according to their appearance in electron microscopy [4], but referring to them by their lineage derivation is more descriptive. The monocytoid intimal synoviocyte, with its equally specialized phenotype that distinguishes it from the typical monocyte exhibits some features found in certain types of dendritic cells.

The monocyte progenitors of the monocytoid intimal synoviocyte enter the intima after leaving blood vessels and differentiate into their mature form in response to guidance clues and interactions apparently provided by the fibroblast-like intimal synoviocytes. The molecules that are responsible for this critical phase of joint histogenesis are beg-inning to be identified. The function and especially the interactions of these two cell types is of special importance in understanding both the biology of the normal joint and in the inflammation of rheumatoid arthritis.

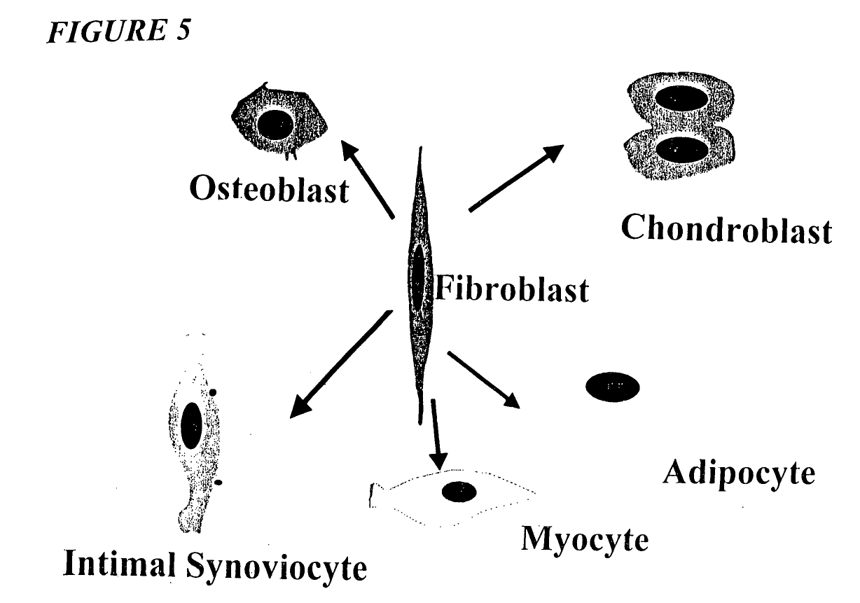

Beneath the intimal lining layer composed of these two cell types lies a thin zone of vascular connective tissue, the subintima that may also contain variable numbers of adipocytes. In contrast to the fibroblast-like intimal synoviocyte, the subintimal synoviocytes appear to be more typical connective tissue fibroblasts. The interrelationships of the various members of the mesenchymal cell-like lineage is depicted in FIG. 5.

The majority of the newly identified genes, including those constitutively expressed at high levels in cultured synoviocytes, appeared very likely to be performing physiologic functions. The result is an emphasis placed on attempting to incorporate the pattern of expression of these genes into schemes that reflect on the normal biology of the joint. For a more detailed and comprehensive treatment, the reader is referred to several comprehensive reviews of the synoviocyte and synovitis for additional information [5-7].

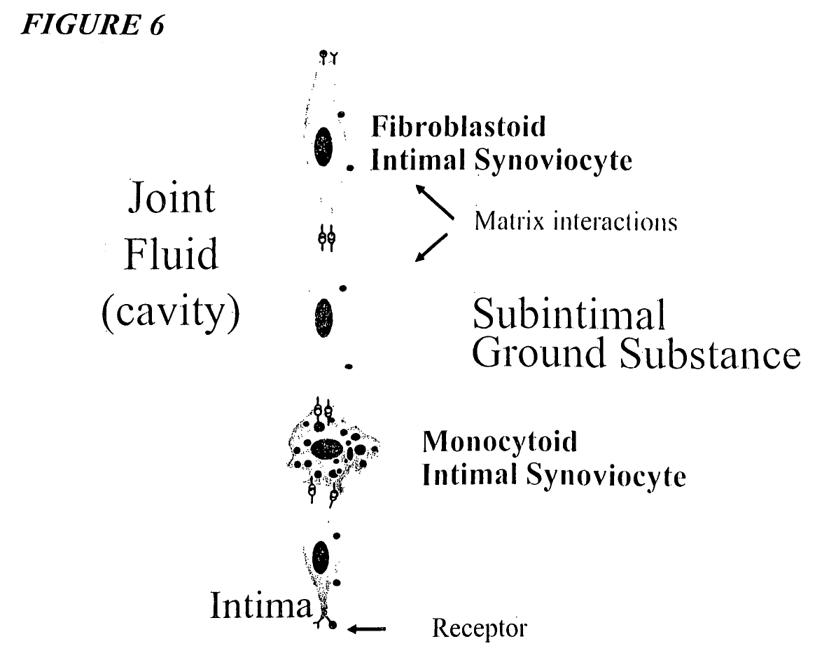

Because the function of the joint is to permit weight bearing motion on avascular cartilage, the lining cells appear specialized for performing a series of physiologic roles aimed at maintaining the integrity of the joint. These functions (table 1) include responsibility for maintaining cartilage viability and function, removal of cartilage debris resulting from impact and weight bearing stresses, and coordinating the immunologic surveillance of this relatively large fluid space. Interestingly, in the instance of the fibroblast-like intimal synoviocyte, these functions require a degree of spatial polarization and organization unusual for a fibroblast and more commonly encountered in an epithelium. One face of the synoviocyte interacts with extracellular matrix fibers and the lining cells, while the other face interacts with the hyaluronate-rich synovial fluid (FIG. 6). The intimal lining, however, has none of the structural features of an epithelium such as a basement membrane or tight junctions.

TABLE 10.1
Partial list of fibroblast-like intimal synoviocyte functions

Surface specialization for:
a) synovial fluid face
b) extra-cellular matrix face of subintima
c) receptors for
1. homotypic (fibroblast-fibroblast)
2. heterotypic (fibroblast-monocyte/macrophage)
Synthesis of components of the synovial fluid and factors for cartilage
nutrition and function
Histogenic functions
guidances clues to monocyte entrance
Immune surveillance
Matrix remodeling
a) metalloproteinases and other proteinases
b) synthesis of matrix components

The functions of fibroblast-like intimal synoviocytes, and their presence in early stages of the development of the joint suggest that these fibroblast-like synoviocytes are responsible for attracting circulating monocyte lineage cells to become resident phagocytic cells in the intima. It is likely that the specific accumulation of monocyte lineage cells in the joint reflects the need for specialized innate immune surveillance and debris removal in this vulnerable mesenchymal cavity. Furthermore, interactions between the monocyte and synoviocyte are responsible for patterning the histogenesis of the normal joint [2]. Similarly, it is possible that the fibroblast-like intimal synoviocyte provides an enhanced recruitment of T- and B-cells into the joint cavity to more effectively perform parallel cognitive immune surveillance functions in this large extracellular space.

Genes essential to physiologic synoviocyte function form the basis of the role of the synoviocyte in disease. Based on the identification of certain genes expressed in fibroblast-like intimal synoviocytes inflammation is a direct consequence of their physiologic role. This is especially evident in the function of genes potentially involved in patterning the histogenesis of the normal joint and the functional adaptations required of fibroblast lineage cells to maintain joint integrity.

An exaggeration of this patterning process, in part attributed to genetic polymorphisms, is involved in the entrance of large numbers of monocytes, macrophages and lymphocytes into the milieu of the rheumatoid arthritis joint, accounting in part for the feedback loops between these cells evident in chronic arthritis. Indeed this potentially proinflammatory surveillance function may suffice to attract autoimmune responses into the joint without the requirement of postulating a drive by a joint-specific autoantigen [8]. Gene programs that are involved in the extensive structural and matrix modifications invoked during embryogenesis of the joint are also involved in development of some of the seemingly aberrant destructive events involving the synoviocytes in rheumatoid arthritis apparently under the paracrine drive of the immune response underlying rheumatoid arthritis.

Lineage, disposition and cell-cell interactions of fibroblast-like intimal synoviocytes. The fibroblast-like intimal synoviocyte appears to be as distinct from a fibroblast as are the other members of this lineage, such as osteocytes and chondrocytes that originate from the same mesenchymal stem cell progenitor as illustrated in FIG. 5. One feature of this differentiation discussed above is that fibroblast-like intimal synoviocytes exhibit a polarization unusual for a typical connective tissue fibroblast, as illustrated in FIG. 6. It is likely that the various surfaces of the intimal synoviocyte are specialized to perform these disparate functions. However it is probable that the receptors for interaction with collagen and other elements of the subintimal ground are disposed only on the abluminal surface of the cell, and that the lateral margins of the intimal synoviocyte exhibit a density of receptors for cell-cell contact, not found on the luminal or abluminal surfaces.

Another lineage feature is that there are two types of cell-cell interactions exhibited by the fibroblast-like intimal synoviocyte. One is the homotypic cell-cell interaction of fibroblast-like intimal synoviocytes with each other and the second is the heterotypic interaction of the fibroblast-like intimal synoviocytes with monocytoid intimal synoviocytes. In electron microscopic studies tight junctions or desmosomes, characteristic of epithelial cells, are not seen suggesting that homotypic and heterotypic cellular interactions during the continued histogenesis of the synovial lining are perhaps entirely receptor-mediated. Additionally, matrix components like collagen VI have been implicated in maintaining cells attached to each other and to the matrix [9]. The expression of these receptors and/or their ligands in the cell are also likely to be polarized.

A third feature is that the fibroblast-like intimal synoviocytes appear responsible for the localization and guidance clues that result in the entrance and differentiation of monocytes in the intima. The property of forming a cell cell relationship with monocytes is another feature that distinguishes the synoviocyte from typical fibroblasts in this lineage. The phenotype of the fibroblast-like intimal synovial lining cell exhibits a phenotype that suggests it also provides receptors for the engagement of counter receptors on the entering monocytoid cells, and that receptor engagement involved in this interaction results in both monocyte adherence and their subsequent differentiation into macrophage-like monocytoid intimal synovial lining cells. In view of the terminal differentiation state of macrophages, there is continual repopulation of the intima by newly entering monocyte lineage cells from the blood.

This patterning, histogenesis and organization of the intimal membrane in joint organogenesis is of particular interest. In fact, it is possible that some of the mechanisms involved in joint development may also be involved in tissue damage during inflammation. During fetal development, cavitation occurs within the primitive skeleton along planes destined to become the articular surfaces of synovial joints. Evidence suggesting that joint cavitation is dependent on the behaviour of fibroblastic cells and/or adjacent chondrocytes, rather than macrophages has been presented in a histochemical study of human fetal limbs [2]. Macrophages are found in the site of the future joint prior to cavitation in the periphery of joint interzones but not at the presumptive joint line in the central interzone, suggesting that macrophages are not actively involved in the process of cavity formation [2]. Uridine diphosphoglucose dehydrogenase (UDPGD) activity was increased in a narrow band of cells at the presumptive joint line prior to cavitation. Since UDPGD activity is involved in hyaluronan synthesis, Edwards has proposed that joint cavitation is facilitated by a rise in local hyaluronan concentration in an area of tissue where cohesion is dependent on the interaction between cellular CD44 and extracellular hyaluronan. It is possible that an early role of macrophages in the histogenesis of the joint is removal of cells that may undergo apoptosis in the formation of the joint cavity, and dysfunction in apoptosis could contribute to synovial hyperplasia, as discussed subsequently.

At a more fundamenental level, the genes involved in the regulation of these and earlier events in joint formation are beginning to be delineated. The mouse brachypodism locus encodes a bone morphogenetic protein called growth/differentiation factor 5. Transcripts of this gene are expressed in a pattern of transverse stripes within many skeletal precursors in the developing limb, corresponding to the sites where joints will later form between skeletal elements. Null mutations in this gene disrupt the formation of many synovial joints in the limb, leading to complete or partial fusions between particular skeletal elements, and changes in the patterns of repeating structures in the digits, wrists and ankles [10]. Particular bone morphogenetic protein family members may also play an essential role in the segmentation process that cleaves skeletal precursors into separate elements. This process helps determine the number of elements in repeating series in both limbs and sternum, and is required for normal generation of the functional articulations between many adjacent structures in the vertebrate skeleton.

Potential role for genes expressed in fibroblast-like intimal synoviocytes as candidates of the genetic susceptibility to rheumatoid arthritis. In terms of the relationship between disease pathogenesis and genetic susceptibility, several of the genes differentially expressed in fibroblast-like synoviocytes from rheumatoid arthritis as compared to osteoarthritis map to non-MHC chromosomal regions where both susceptibility loci for rheumatoid arthritis and experimental arthritis in rodents are located. This makes these genes candidate susceptibility genes whose expression may be regulated differently in alternate gene forms. It is a distinct possibility that normal down-regulatory pathways that operate to protect the joint from going into a state of persistent inflammatory and local immune response are deficient in patients with rheumatoid arthritis.

Problems in the study of synoviocytes. The two fundamental questions asked of the fibroblast-like intimal synoviocyte are: a. what genes are expressed that enable it to perform its distinctive functions and how does this pattern of gene expression differ from that of the typical fibroblast and from the subintimal fibroblast-like synoviocyte? b. How is this pattern of gene expression altered in inflammation and what are the functional consequences of this change? This, because of several features of the biology of both the normal and the inflamed joint, including the fact that the single layer of fibroblast-like intimal synoviocytes is not separated from the subintima by a basement membrane, (FIG. 2) making the isolation of fibroblast-like intimal synoviocytes from the normal joint a difficult problem.

Accordingly, because of the uncertainty of whether a given fibroblast-like cell propagated in tissue culture originated from the intima or subintima, cells cultured from the joint are referred to as fibroblast-like synoviocytes. In rheumatoid arthritis and many other chronic arthritides the synovial intimal membrane becomes highly hyperplastic, forming multiple layers of cells that in short term cultures exhibit a stellate or dendritic-like phenotype. However, despite the greatly, increased number of fibroblast-like intimal synoviocytes, the same anatomic problem persists of the inability to reliably distinguish the origin of a cell from the intima or subintima.

The second major problem is that the inflammatory state has induced additional alterations in phenotype by what is likely to be a paracrine mechanism. These cytokines and growth factors may be either directly derived from infiltrating lymphocytes or reflect additional activation pathways involving monocytes and/or fibroblast-like cells. By definition, these paracrine effects are short lived and disappear in culture after several days. It however has been hypothesized, but not established, that a consequence of prolonged exposure to these paracrine effects may persist leaving a phenotypic immunologic imprinting that could account for a significant percentage of the phenotypic behavior of these cells in culture [11, 12].

Nevertheless, many studies use inflammatory synoviocytes as a starting point, particularly after they have been cultured in an attempt to isolate them from the short-lived monocytoid cells and allow their phenotype to recover from most, if not all, of the paracrine effects of exposure to the products of an immune response.

Distinctive phenotype of cultured synoviocytes from inflammatory synovitis. In freshly enzyme-dissociated preparations of cells obtained from rheumatoid arthritis synovia and those from other inflammatory arthritides, many fibroblast-like lineage cells are found that have a striking stellate or dendritic morphology. The striking HLA-DR expression [15, 16] and morphologic phenotype of the fibroblast-like synoviocyte freshly isolated from a rheumatoid synovium has sometimes warranted the term dendritic cell although this cell is also referred to as a stellate cell (FIG. 3). The use of the term dendritic brings up the question of whether these cells exhibit a functional relationship to the dendritic cells of the myeloid and lymphoid lineages that are increasingly being recognized to play a key role in the early events of the immune response. These cells lack the property of enhanced endocytosis or phagocytosis, expression of CD14, Fc receptors and the leukocyte common antigen, CD45 [17], making it very likely that they belong to the fibroblast lineage and are most probably fibroblast-like intimal synoviocytes. When preparations of these cells are placed into culture, the preponderance of these cells loose expression of HLA-DR, but the stellate morphology remains, strongly suggesting an astrocyte morphology. However, the precise lineage and fate of the cells that express massive amounts of HLA-DR molecules have not been carefully traced, particularly with their relationship to the stellate synoviocytes that characterize rheumatoid arthritis samples and are most likely to be fibroblast-like intimal synoviocytes. Moreover, it is not known whether these cells are efficient antigen-presenting cells. Zvaifler, et al. have also emphasized the increased percentages of true dendritic cells in the joint [18].

During the first three passages of these cells many of the marked phenotypic alterations such as the expression of MHC class II molecules greatly diminishes, emphasizing the role of paracrine and cell interaction factors in inducing some of the phenotypic alterations found in the freshly isolated synoviocytes [17, 19]. However, the synoviocytes obtained from synovial tissue of individuals with rheumatoid arthritis [4, 20, 2.1] and other disorders with marked degrees of intimal hyperplasia do not revert to an entirely typical fibroblast-like morphology and behavior, maintaining a complex phenotype that includes varying degrees of stellate morphology, enhanced growth, increased glucose consumption, altered adherence behavior, constitutive overproduction of metalloproteinases and the elaboration of proinflammatory cytokines [14, 15, 22, 23], as well as loss of contact inhibition [20].

The distinctive but not entirely uniform phenotype of the remaining cultured rheumatoid synoviocytes is not found in similarly cultured synoviocytes obtained from osteoarthritis synovia that have been shown to lack lining cell hyperplasia and any inflammatory cell infiltration [14, 24] (Winchester unpublished observations). There is postulation that the distinctive changes in synoviocyte phenotype observed in these cultured cell lines mirror certain similar events occurring in the inflamed synovium itself [11, 13, 22, 23, 25].

The pattern of gene expression in these cultured cells has been characterized in a series of studies [8, 11, 14, 26]. Two features of the cells exhibiting the distinctive phenotype were identified: The first is that cells exhibiting this phenotype are not specific for rheumatoid arthritis, as it is also demonstrable in cultures initiated from a number of different entities characterized by chronic inflammation, including psoriatic arthritis and cases of what was termed osteoarthritis, but presented considerable degrees of inflammation [13, 14]. The second feature is that the pattern of gene expression is similar, but not at all identical among samples from different inflammatory synovia.

There are four possible explanations for the distinctive phenotype and function of these long term cultured cells (table 2). Each of these possibilities has a different implication in terms of whether the genes found to be overexpressed in these cultures are identifiable in the normal synovium.

TABLE 10.2
Four possible explanations for the distinct phenotype of rheumatoid
arthritis cultured synovial fibroblasts

Disease-specific sustained modulation in gene expression
Local paracrine regulation
Phenotypic ‘imprinting’
Transformed cells
Secondary to unidentified viral infection
Normal intimal cell phenotype
Differences represent different percentages of intimal versus subintimal
cells in the synovial tissue
Normal intimal cell phenotype and is dependent on genetic polymorphisms
arthritis susceptibility genes

First, the phenotype could be a consequence of a disease-specific sustained modulation in gene expression in the intimal and subintimal fibroblast lineage cells of the joint that developed as a response to prolonged paracrine signaling through products of a local immune response as has been postulated in earlier work [11, 13, 14, 27]. This sustained modulation, analogous to a phenotypic imprinting process, would be clearly distinct from the paracrine mediated activation phenotype in that it does not decay quickly in culture. Rather the phenotype would be maintained as a sustained pattern of altered gene expression through many months of culture. The implication of this pattern is that neither normal fibroblast-like intimal synoviocytes, nor subintimal synoviocytes would have increased expression of genes found in both of these cell types in rheumatoid arthritis.

A second possibility is that the cells are primarily transformed as suggested by Gay and colleagues [28], where there would be a disease specific nature of the distinctive phenotype. This viewpoint considers the disease of rheumatoid arthritis to result from an immune response against the agent responsible for the transformation, or a possible innate abnormal regulation of oncogene expression leading to a hyperproliferating cell. The lack of disease specificity for the phenotype renders this otherwise attractive possibility more remote. The implication of this model is that normal fibroblast-like intimal synoviocytes and subintimal synoviocytes would not express these genes, and that it is likely that the overexpression pattern is specific for rheumatoid arthritis and not other chronic synovitides.

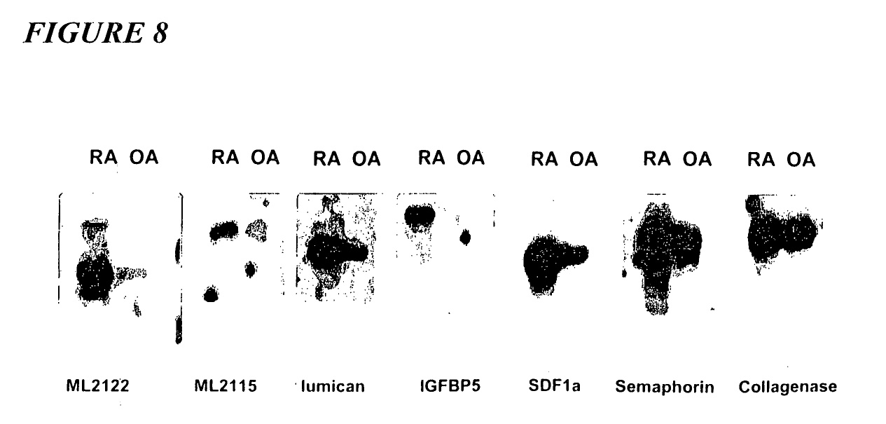

Thirdly, the distinctive phenotype observed in these cultures could be the normal phenotype of the fibroblast-like intimal synoviocyte found in the normal joints in all individuals [8]. The differences in cultural phenotype between inflammatory and non inflammatory synovitis would simply reflect the increased proportion of fibroblast-like intimal synoviccytes compared to subintimal synoviocytes in the starting culture material obtained from a joint with intimal hyperplasia, as illustrated in FIG. 8. By parsimony, this more prosaic concept is the simplest of the explanations. The implication here is that normal fibroblast-like intimal synoviocytes, but not subintimal synoviocytes would express the genes found constitutively increased in cultured rheumatoid arthritis synoviocytes. In synovitis, the hyperplasia of fibroblast-like intimal synoviocytes would lead to a relative increased expression of genes that are normally more characteristic of the mesenchymal stem cell.

The fourth possibility is essentially a variation of the third, with the important distinction that while the over expression phenotype reflects the starting phenotype of the individuals pre-arthritis intimal synoviocytes, this phenotype of genetically determined increased expression is intrinsically abnormal because of the presence in the allele of a regulatory polymorphism that predisposes to immunologically-mediated arthritis. Thus, genes that are over- or under expressed in the intimal synoviocyte in arthritic disease are candidate genes for genetic polymorphisms defining the susceptibility state. A variation on this last possibility is the situation where the genetic abnormality arises somatically through mutation, rather than through the inheritance of alternate forms of germline genes. The implication of this model is that in the patient, unaffected joints containing normal fibroblast-like intimal synoviocytes would over express these genes before arthritis developed, but that the overexpression of these genes would be lacking, or reduced in entirely normal individuals without rheumatoid arthritis.

Strategy for the identification of genes responsible for the distinctive phenotype and their relationship to intimal and subintimal synoviocyte lineages. To identify the genes responsible for the distinctive phenotype of the cultured synoviocytes obtained from a rheumatoid arthritis patient, a gene discovery approach has been taken that involves identifying genes similarly and differently expressed compared to a line derived from a selected osteoarthritis sample. Our recent approach was based on the construction of representational difference libraries [29, 30] that had been used to clone the differences between two complex genomes. It involves a cloning procedure with PCR amplification of cDNA to generate simplified representations of the expressed genes followed by a modified subtractive step and subsequent screening to facilitate the gene identification.

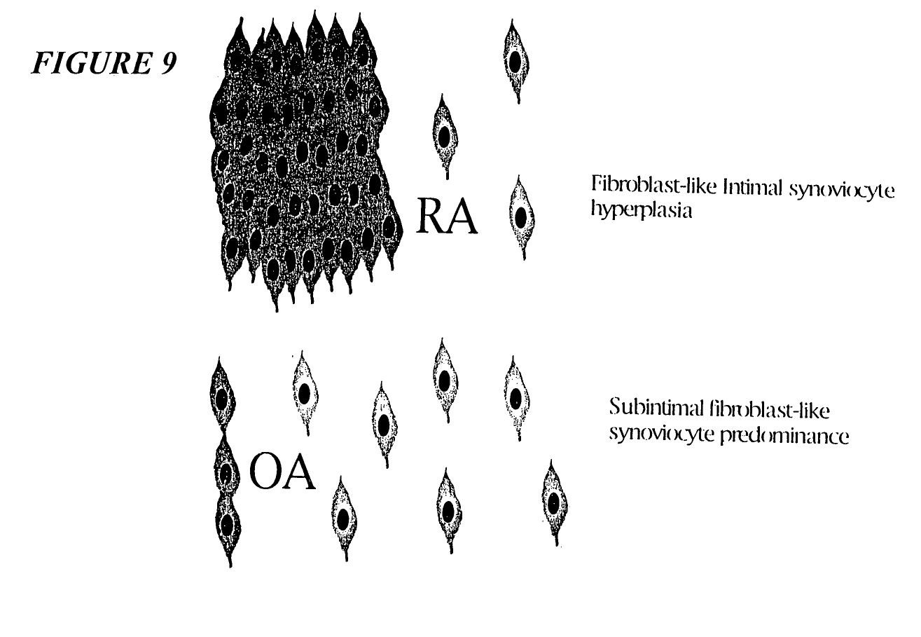

A number of genes were identified in cultured synoviocytes obtained from both rheumatoid arthritis and osteoarthritis that were expressed at approximately the same or similar levels in each parent cell line on Northern or equivalent analyses. In light of the possibilities explaining the basis of the distinctive phenotype, we interpret these genes as being expressed in both fibroblast-like intimal and in subintimal synoviocytes. In contrast, other genes exhibited increased expression by Northern analysis in only the rheumatoid arthritis synoviocytes (FIG. 9). Interpretation of this result implies that the genes found constitutively overexpressed in the rheumatoid arthritis synoviocyte culture are expressed at high levels in fibroblast-like intimal synoviocytes and are primary markers of the phenotype of this lineage (table 3).

TABLE 10.3
Genes identified through a subtraction method differentially expressed
in RA and OA fibroblast-like synovial cultures and correlation
with their possible lineage origin

| Genes preferentially | Genes expressed in both RA |
|---|---|
| expressed in RA synovial cultures: | and OA synovial fibroblast culture |
| intimal origin | intimal and subintimal origin |
| Biglycam | Adrenomodulin |
| IFN-induced 56 Kd | subunit of GsGTP binding protein |
| IFN-induced 71 Kd | .-B-crystallin |
| 2′5′-oligoadenylate synthetase | |
| IGF-BP5 | B94 protein |
| Lumican | beta subunit of prolyl-4-hydroxylase |
| Mac2-binding protein | Candidate sulphatase |
| ML2115 | Cathepsin B |
| ML2122 | Collagen alpha 1 type III |
| SDF-1a | Collagenase IV |
| Semaphorin VI | Complement C1r |
| VCAM-1 | Complement C1s |
| | Complement factor B |
| | DNA-binding protein TAXREB10 |
| | Elongation factor 2 |
| | Epithelin |
| | Extracellular protein (SI-5) |
| | HLA-E heavy chain |
| | Interferon- IEF SSP 5111 |
| | Manganese superoxide dismutase |
| | Milk fat globule protein |
| | Muscle fatty acid binding protein |
| | NMB protein |
| | Osteoblast specific factor 2 (OSF-2) |

Considering the pattern of gene expression to reflect cell lineage is the least biologically complex of the possible interpretations. However, whether this interpretation is correct for some or all of the identified genes will be determined by the results of studies designed to characterize their expression on normal and inflamed joint tissue samples, specifically distinguishing intimal from sub-intimal cells. At this moment immunophenotypic distinction between these two cell lines, or a simple method to differentiate them remains the subject of ongoing studies.

The specialized functions of the fibroblast-like intimal synoviocytes are mediated by either quantitative differences in the expression of genes found on other members of the fibroblast-like lineage, or the qualitative expression of genes unique to the synoviocyte sublineage. The genes identified by the subtraction library method as differentially expressed in rheumatoid arthritis synoviocyte cultures are likely candidates for comprising the fibroblast-like intimal synoviocyte phenotype [8]. These include differentially expressed chemokines like SDF-1, connective tissue matrix components like biglycan and lumican, adhesion molecules like VCAM-1 and other molecules of a less clear function in the synovial tissue like semaphorin VI, Mac2-binding protein, IGF-BP5, interferon-inducible 56 kd protein and interferon-induced 71 kb 25 oligoadenylate synthetase (table 3). Additionally, two genes that were not homologous to any known genes were overexpressed in rheumatoid arthritis Synovial fibroblast-like cultures (ML2122 and ML2115).

VCAM-1 had previously been identified by Edwards as being expressed by normal fibroblast-like intimal synoviocytes [2, 6], and this observation supports the interpretation that the remaining genes found by the subtraction method are characteristic of fibroblast-like intimal synoviocytes. In addition, several other genes have been identified as being selectively, or more highly, expressed by fibroblast-like intimal synoviocytes, either from staining patterns in normal or diseased synovial membranes or from cultured cells. These include other chemokines such as IL-8, RANTES, MCP-1 [8, 31-33], cytokines like TNF-α, IL-1, IL-6, GM-CSF [14, 34, 35], IL-11 [36, 37], metaloproteinases and other proteinases [13, 38, 39], adhesion molecules like ICAM-1 [40-44], integrins [45] and CD44 [43, 46, 47] and co-stimulatory molecules like CD40 [48] (Winchester R., unpublished observations) (table 4).

A number of genes were similarly expressed in the rheumatoid arthritis and osteoarthritis synovial fibroblast cultures, and likely are genes expressed by both cultured fibroblast-like intimal and sub-intimal synoviocytes. Among these genes are some involved in cellular and matrix turn-over like collagenase IV, genes involved in the inflammatory response like manganese superoxide dismutase, complement factor B, interferon-γ IEF SSP 5111 and HLA-E heavy chain, and other genes with unknown function in the synovium like NMB protein, α-B crystallin, B94 protein and muscle fatty-acid-binding protein. Other genes have been shown to be either similarly expressed in synovial fibroblasts from patients with a variety of diseases or specifically expressed in both intimal and subintimal layer by in situ hybridization or staining with monoclonal antibodies (tables 3 and 5).

Chemokines, Cytokines and Growth Factors

Chemokines D The finding that a number of chemokines have been identified as being likely overexpressed by the fibroblast like intimal synoviocytes, including IL-8 [49], Gro-α [50], MCP-1 [31, 49], MIP-1α [51] and SDF-1 [8] directs attention to the role of these molecules in the normal synovium Moreover, it is of interest whether they could participate in fostering the localization or intensification of an autoimmune or immune response into the joint.

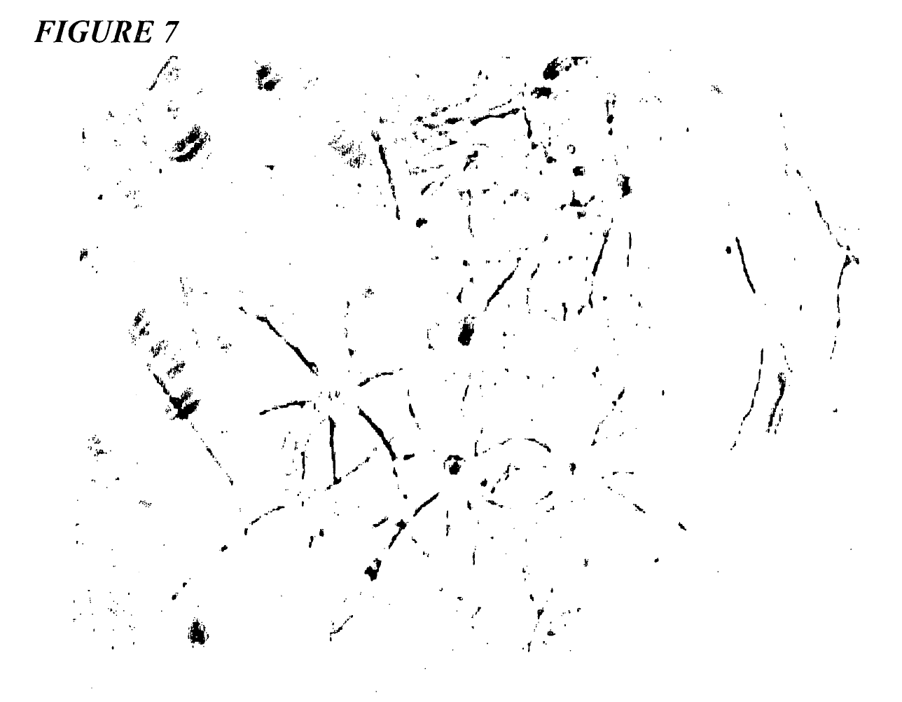

Expression of chemokines and their receptors has been demonstrated to have a critical role in the regulation of the attachment of leukocytes and endothelial cells, and in their passage into the tissues [52, 53]. For instance, leukocyte egress from blood vessels occurs in four identifiable stages [54] (FIG. 7). During most of these stages, chemokine receptor expression in the leukocytes is critical in regulating directed leukocyte egress.

Among chemokines, SDF-1 is one of the most efficacious in T cell and monocyte migration [52]. Both CD4+ and CD8+ cells, as well as CD45RA+ naive and, less effectively, CD45RO+ memory T-lymphocyte subsets in peripheral blood are subject to SDF-1 chemoattractive effects [55]. Similarly, monocyte-lineage dendritic cells acquire CXCR4 upon induction with GM-CSF and IL-4 [56]. Additionally, SDF-1 is an important B cell developmental and maturation factor, as revealed by the observation that mice lacking SDF-1 show defects on B-cell lymphopoiesis and bone marrow myelopoiesis [57].

The SDF-1 receptor, CXCR4, is a seven-transmembrane-spanning, G-protein-coupled receptor and is a co-receptor for T-cell-line tropic human immunodeficiency virus HIV-1. CXCR-4 is constitutively expressed by quiescent, resting EC. A similar phenotype was identified in both SDF-1 and CXCR4 knock-out mice [57, 58]. Cytokine stimulation studies revealed that bFGF upregulates endothelial CXCR-4 expression, whereas TNF-α downregulates it. In addition to the abnormalities seen in the SDF-1 knock-out, mice lacking CXCR4 also have defective formation of the large vessels supplying the gastrointestinal tract, defective in vascular development, and show many proliferating granule cells invading the cerebellar anlage, suggesting the involvement of the SDF-1/CXCR4 system in neuronal cell migration and patterning in the central nervous system.

The chemokine receptors CXCR3 and CCR5, recently described to be preferentially expressed in Th1 T cells [59], are expressed in the majority of the synovium infiltrating T cells in rheumatoid arthritis [60]. In contrast, CXCR4, the SDF-1 receptor, does not appear to be preferentially expressed in Th1 or Th2 cells, but is preferentially expressed on na•ve cells. IP-10, one of the chemokine ligands for CXCR3, is produced by synovial fibroblast cultures in response to IL-1 and TNF [61]. Mig, another CXCR3 binding chemokine, has not been studied in the rheumatoid synovium. The CCR5 chemokine ligands RANTES [33, 62], MIP-1α [51, 62] and MIP-1β [62] have been described as overexpressed in the rheumatoid arthritis synovium. Furthermore, antibodies to RANTES ameliorated adjuvant-induced arthritis in rats [63] The production of chemokines by synovial fibroblasts, the fact that the infiltrating lymphocytes express the corresponding receptors and are likely Th1 cells, and the improvement of arthritis in experimental animal models using anti-chemokine therapy support the concept that chemokines have an important role in the localization of T cells and the inflammatory process to the synovial membrane.

We speculate that the production of SDF-1 by fibroblast-like intimal synoviocytes in the normal joint could act as a guidance cue for the continual entrance into the intimal synovial membrane of monocyte lineage precursors committed to differentiation into phagocytic lining cells or to progress through normal differentiation pathways [8]. Similarly SDF-1 and other chemokines elaborated by the normal synoviocytes may act to enhance the ingress of lymphocytes into the joint tissues to facilitate increasing innate and acquired immune surveillance in the synovial cavity. These same mechanisms may be relevant to the immune response of rheumatoid arthritis in two ways: first, the chemokines may attract an autoimmune response in the synovial intima, although it is not driven by an antigen uniquely expressed there. Second, the chemokines may modify the responsiveness and organization of the ongoing autoimmune response and cells related to it such as dendritic cells. Furthermore, since these genes are constitutively expressed as part of normal physiologic properties of the cells, it is likely that regulatory or suppressor mechanisms exist to normally protect the joint from developing a chronic inflammatory process. Abnormalities in those regulatory pathways, especially those that occur through genetic polymorphisms could have a fundamental role in disease susceptibility.

TABLE 10.4
Genes differentially expressed in cultured rheumatoid fibroblast-like
intimal cells, their chromosomal location, their relationship with
susceptibility loci mapped in rheumatoid arthritis and with
homologous loci regulating experimental arthritis in rats.

| | | RA | |
|---|---|---|---|
| | Human | susceptibility | Arthritis |
| Genes | chromosome | loci^{e} | loci in rats |
| Cytokines | | | |
| IL-1 | 2q12 | | |
| IL-6 | 7p14 | | Cia3, Aia3 |
| IL-11 | 19q13.3-q13.4 | | Cia2 |
| IL-15 | 4q31 | | |
| TNF-α | 6p21.3-21.1 | Cornelis | Cia1, Aia1, |
| | | | Oia1 |
| GM-CSF | Sq23.1-23.3 | | |
| TGF-β | 6p11.1 | | |
| Chemokines | | | |
| SDF-1 | 10q11.1 | | |
| RANTES | 17q11.2-q12 | | |
| MCP-1 | 17q11.2-q12 | | |
| MIP-1α | 17q11-q21 | | Cia5, Oia3 |
| IL-8 | 17q24.2-24.3 | | Cia5, Oia3 |
| MMP^{d}, proteases and | | | |
| inhibitors | | | |
| MMP-1 (collagenase) | 11q14.2 | | Cia2 |
| MMP-2 (gelatinase A) | 16q 12.1 | | |
| MMP-3 (stromelysin) | 11q14.2 | | Cia2 |
| MMP-10 (stromlysin 2) | 11q14.2 | | Cia2 |
| MMP-13 (collagenase 3) | 11q14.2 | | Cia2 |
| Cathepsin B | 3p21.1 | | |
| Cathepsin L | 9q21.2 | | |
| Cathepsin S | 1q21.1 | | |
| TIMP-1 | Xp11.4-q11.2 | | |
| TIMP-2 | 17q25 | | |
| Oncogenes | | | |
| and transcription factors | | | |
| Myc | 8q24, 12-q24,13 | | Cia3 |
| Fos | 14q22-q23 | | |
| Jun | 1p31.1-22.3 | | |
| NF-kappaB p50/p65 | 4q24/11q13 | | |
| Matrix compounds | | | |
| Biglycam | Xq28 | Cornelis | |
| Lumicam | 12q21.3 | | Cia8^{f} |
| Adhesion Molecules | | | |
| VCAM-1 | 1p13.3-p11 | | Cia10^{c} |
| CD44 | 11pter-p13 | | |
| Others | | | |
| CD40 | 20q12-q13.2 | | |
| HLA-E | 6p21.3-p21.1 | Cornelis | Cia1, Aia1, |
| | | | Oia1 |
| Igf-bp5 | 2q33-q34 | | |
| Mac-2BP | 17q25 | | |
| Semaphorin VI/E | 7q21 | | Aia2 |
| IFN-inducible 56 kd^{b} | 10q22.3 | | |
| IFN-inducible 71 kd 2′5′ | 12q21.3-q22 | | Cia8^{f} |
| oligo a.s.^{b} | | | |

a Cia = collagen-induced arthritis; Aia = adjuvant-induced arthritis; Oia = oil-induced arthritis.
^{b}IFN = interferon; oligo a.s. = oligoadenylate synthetase.
^{c}Suggestive of linkage;
^{d}MMP = matrix metalloproteinase, TIMP = tissue inhibitor of metalloproteinase.
^{e}Cornelis = Cornelis et al. 1998;
^{f}= Dracheva et al., unpublished observations.

TABLE 10.5
Partial list of genes expressed by fibroblast-like synoviocytes^{a}

Matrix components
Collagen I
Collagen III
Collagen IV
Biglycam
Laminin
Lumicam
Perlecam
Hyaluronan
Fibronectin
Proteoglycans
Glycosaminoglycans
Metalloproteinases, other proteinases
and inhibitors
MMP-1 (collagenase)
MMP-2 (gelatinase A, collagenase 4)
MMP-3 (stromelysin)
MMP-10 (stromelysin 2)
MMP-13 (collagenase 3)
Cathepsin B
Cathepsin L
TIMP-1
TIMP-2
Cell-cell, cell-matrix interactions,
receptors
Mac2-binding protein
VCAM-1
ICAM-1
ICAM-2
alpha-alpha6 integrins
beta1, beta4 integrins
PECAM/CD31
CD44
IGF-BP5
Cadherin-11
Plasminogen receptor
Cytokines and growth factors
IL-1
IL-6
IL-15
IL-11
TNF-α
GN-CSF
bFGF
TGFb
PDGF
Semaphorin VI/E
Oncostatin M
LIF
Chemokines
SDF-1
MIP1α
MCP-1
IL-8
RANTES
GRO-α
Co-stimulatory molecules
CD40
Complement
Complement C1r
Complement C1s
Complement factor B
HLA genes and activation markers
HLA-A, B, C
HLA-DR
HLA-DQ
HLA-E heavy chain
Interferon-gamma IEF SSP 5111
IFN-induced 71 kDa 2′5′-oligoadenylate
synthetase
IFN-induced 56 kDa
Oncogenes and transcription factors
NF-kappaB
c-Jun
c-Fos
Sis
c-Myc
Egr-2
Ras
Apoptosis regulatory genes
p53
FAS
Bcl-2
Other inflammation-related genes
Manganese superoxide dismutase
Cox1
Cox2
PGE-2
Neurohormones
Substance P
Parathyroid hormone
Others with unclear function in the
synovium
Adrenomodulin
α subunit of GsGTP binding
protein
α-B-crystalline
B94 protein
β subunit of prolyl-4-hydroxylase
Candidate sulphatase
DNA-binding protein TAXREB10
Elongation factor 2
Epithelin
Extracellular protein (SI-5)
Milk fat globule protein
ML2115
ML2122
Muscle fatty acid binding protein
NMB protein
Osteoblast specific factor 2
(OSF-2)
^{a}The character of this chapter does not permit the coverage of all the genes expressed by synovial fibroblasts. Most of these genes are referenced in the text.

Increased expression of several cytokines including TNFα, IL-1, IL-6, IL-11, IL-15, GM-CSF, TGFβ, PDGF and bFGF by fibroblast-like intimal synoviocytes has been demonstrated [13, 14, 35-37, 64-69].

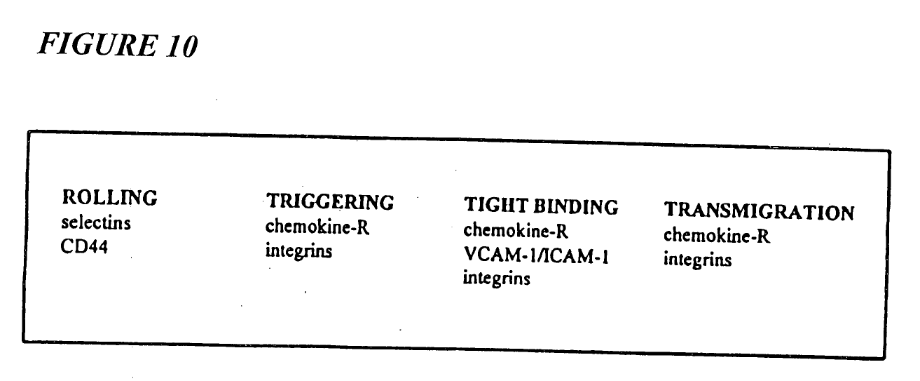

Cytokines have key functions in altering the pattern of gene expression and consequently cell function at several levels in the activation of the endothelium, in the regulation of the immune response, including immune deviation between TH1 and TH2, and on cells in the area of an onging immune response. Some of these effects are on cell-cell and cell-matrix interactions, while other effects may affect different cell functions. Some of these alterations mediate cartilage damage The migration of a given inflammatory cell into the synovium is not a random event but one determined by the prior immunologic history of the particular cell as well as that of the endothelium. The increased production of certain cytokines like IL-1 and TNF-α has been shown to activate the endothelium and to initiate overexpression of certain adhesion molecules. This would affect phases 2, 3 and possibly 4 in the diagram of directed cellular egress (FIG. 10). Accordingly, the increased activation status of the endothelium in concert with increased chemokine production could highly facilitate the localization of inflammatory and autoimmune cells to the synovial membrane, further perpetuating the disease process and tissue injury. A similar failure in the normal suppressor regulatory pathways proposed above could also operate to release an unopposed pro-inflammatory cytokine production. Some selected examples follow:

IL-1 induces the expression of more IL-1α and IL-1β, in a positive feedback loop [70]. IL-1 also induces transcriptional activation of protein kinase C, and by a separate pathway induces the synthesis of PGE₂ [71]. This latter response is much greater when rheumatoid arthritis fibroblast-like cells are used instead of cells from osteoarthritis synovia and the response is potentiated by PDGF and certain other polypeptide growth factors, suggesting that fibroblast-like intimal synoviocytes are the source of increased amounts of this cytokine [72]. These two pathways initiated by IL-1 also converge to regulate the transcriptional activation of stromelysin. IL-1 induces fibroblast-like synovial cell lines to increase IL-6 gene expression, by an incompletely defined pathway that is suppressed by corticosteroids [68]. Similarly IL-8 expression is induced by the addition of IL-1 or TNFα [73], although to much lower levels than those elaborated by synovial monocytoid cells from rheumatoid arthritis samples. IL-1 induces the expression of GM-CSF mRNA with a maximum at 4 hours [66]. IL-1 also induces the production of fibronectin and types I and III collagen [74].

TNFα is produced by fibroblast-like intimal and subintimal synoviocytes, and by synovial monocytes/macrophages, and among several functions it is capable of inducing fibroblast-like intimal synoviocytes cellular proliferation, matrix metalloproteinases (MMP) [75] and cathepsin production [76]. As with IL-1, the addition of TNFα to fibroblast-like cell cultures induces the expression of GM-CSF [66]. A number of studies have documented the importance of TNF in the development of arthritis, including a TNF-transgenic mouse that develops chronic arthritis [77], and the significant improvement of disease with agents targeting TNF [78-80].

IL-6, a cytokine with effects on B-cell differentiation, is constitutively expressed in synovial fibroblasts obtained from rheumatoid arthritis patients [14, 34]. This cytokine appears critical to the development of a pathway leading to arthritis in mice, as gene-targeted mutation prevents disease [81]. IL-6, particularly in the presence of soluble IL-6 receptor, induces synovial fibroblast proliferation and IL-1 production [82]. However, IL-6 does not appear to directly induce MMP expression [83]; On the contrary, IL-6 can a potent inducer of TIMP-1 [84, 85]. IL-6 regulates osteoclast activity and through this mechanism perhaps participates in the bone loss and in the erosive process seen in RA. Other molecules of the IL-6 family like oncostatin M, leukemia inhibitory factor (LIF) and IL-11 have been reported by other groups to be differentially expressed in synovial fibroblasts of rheumatoid arthritis compared to osteoarthritis [67, 86], again suggesting that they are constitutively produced by fibroblast-like intimal synoviocytes. Oncostatin M, but not IL-6 or LIF, increased MMP-1 [87], particularly in the presence of IL-1 [88]. In addition, Oncostatin M [88] and LIF, like IL-6, are able to induce TIMP-1 expression in synovial fibroblasts [85].

Interestingly, both IL-6R and LIFR map to an interval in the human genome syntenic to an interval where a non MHC arthritis severity regulatory locus has been mapped in collagen-induced arthritis, suggesting that these genes are candidate susceptibility/severity genes [89].

IL-15 is another cytokine produced by synovial fibroblasts (McInnes, personal communication) capable of activating T cells in the absence of IL-2, as well as inducing TNFα production [90]. Furthermore, it is also a potent chemotactic factor for leukocytes to migrate into the synovial membrane [69, 90]. GM-CSF, a growth-factor produced by fibroblast-like intimal synoviocytes, is important for the maturation and homing of macrophages and dendritic cells, and it is produced by normal and rheumatoid arthritis fibroblast-like intimal synoviocytes [13, 91]. A recent study of collagen-induced arthritis in GM-CSF knock-out mice described significant resistance to disease, despite evidence for T and B cell-mediated autoimmune responses [92]. This suggests that GM-CSF, like SDF-1 and other cytokines and chemokines produced by the fibroblast-like intimal synoviocyte, may not be necessarily directly involved in the genesis of the autoimmune response, but instead, operate to localize this systemic autoimmune response to the joint.

TGF-β is widely distributed in the rheumatoid synovium, predominantly located in the lining cell layer and in the perivascular lymphoid aggregates. Both fibroblast-like and monocytoid lineage cells expressed this growth and immunoregulatory factor [93]. If TGF-β is synthesized in an attempt to downregulate the inflammatory and destructive processes it apparently does not fully succeed in this task.

The overexpression of the semaphorin VI, human homologue of mouse semaphorin. E by synovial fibroblasts [8, 94] is intriguing because the semaphorins are a family of transmembrane signaling and secreted guidance glycoprotein molecules that are implicated in directing axonal extension and operate broadly in neuronal patterning [95]. However, in view of the relatively small number of axons in the synovium, it seems unlikely that the physiologic role of the semaphorin VI molecule is to signal through an axonal receptor. Indeed its expression has been observed in a wide variety of tissues such as the heart, skeletal muscle, colon, small intestine, ovary, testis, and prostate. This suggests strongly that the semaphorins may have a function other than in guidance of axon, and preliminary evidence suggests that semaphorin VI plays some role in chemotaxis of monocytes and their differentiation. Its function on fibroblast-like cells is to be determined. It is noteworthy to point out that neuropilin, a receptor for semaphorin, is expressed on vascular endothelial cells. Neuropilin expression is upregulated by TNF-α and implicated in angiogenesis as a co-receptor of VEGF. Previously, other molecules initially identified in the central nervous system were found in the synovium and described as having pro-inflammatory properties, like substance P, CRH and others [⁹⁰]. Therefore, it seems reasonable to speculate that in addition to chemotaxis, semaphorin VI may also have a direct role in the local inflammatory process. Semaphorin may, however, play other roles as its identity as a multidrug resistance element and loss in certain tumors suggests. [97, 98]

Several molecules expressed by fibroblast-like intimal synoviocytes appear to be candidates for mediating cell-cell interactions involved in the histogenesis of the normal synovium. These include monocytoid-fibroblast-like and fibroblast-like-fibroblast-like synoviocyte interactions. Some of these genes have a well defined role in cell-cell interaction, while others have the potential to act as cell interaction receptor-ligand systems, but also have other actions.

Among the well recognized adhesion molecules and receptors differentially expressed in the rheumatoid fibroblast-like intimal synoviocytes are VCAM-1, a 110 kd, member of the immunoglobulin gene superfamily, and Mac-2 binding protein (Mac-2BP), also termed 90 k tumor associated protein. Both VCAM-1 and Mac-2BP exhibit properties that suggest they could mediate heterotypic and homotypic binding of monocyte-lineage intimal synoviocytes to fibroblast-like intimal synoviocytes VCAM-1 has been previously described as markedly increased on rheumatoid arthritis synoviocytes [6, 25] and it binds circulating monocytes and lymphocytes expressing the α4§1 (VLA-4) integrin.

Mac-2BP, a heavily N-glycosylated secreted protein which binds stoichiometrically to the macrophage associated lectin Mac-2 (galectin-3) [99, 100], has been shown to increase in the serum of cancer and HIV positive patients, suggesting an implication of its participation in some aspects of immune reaction. Mac-2BP also binds to the monocyte CD14 structure in the presence of LPS and LPS-binding protein. [101]. Binding of Mac-2BP to these receptors initiates monocyte lineage cells to secrete IL-1 [102].

Similarly, these molecules could attract and facilitate interaction with and activation of monocytes. For example, Mac-2BP that induces homotypic monocyte aggregation and activation [100] could be a factor present in supernatants from cultured rheumatoid arthritis synoviocytes that induces blood monocytes to form giant cells [103]. Thus, along with the variety of genes that mediate the well recognized effector functions of matrix remodeling and tissue destruction [74], the genes expressed by the mesenchymal cells of the joint may affect antigen non specific immune localization or amplification mechanisms that could play a role in the puzzling phenomenon of why localized joint inflammation develops in many disparate diseases in the setting of immune responses that apparently have little to do with the joint.

Cadherin-11 was recently reported to be expressed in rheumatoid arthritis synovial fibroblasts [104]. Cadherin may participate in the mediation of homophilic adhesion between synoviocytes. All of the cell-cell interaction have the important potential of reverse signaling which could influence synovial proliferation and pannus invasion into cartilage or could engage in a heterophilic interaction that anchors lymphocytes within the synovial membrane.

In view of the unusual situation of the intimal synoviocyte to delimit a fluid environment from a typical connective tissue matrix, the typical fibroblast-matrix interactions with collagen and other fixed fibrillar structures occur in a polarized manner on one side of the cell, and likely necessitate a polarized localization of the gene products. Although the precise polarization of gene products has not been studied, several matrix component genes exhibited a pattern of expression suggesting that they are constitutively produced by fibroblast-like intimal synoviocytes. Lumican was identified as likely to be constitutively expressed by fibroblast-like intimal synoviocytes. [8]. Lumicans role in the synovium is not understood. However, its role in corneal transparency [105], and in inhibition of macrophages adhesion to intact corneal keratan sulfate proteoglycans are of interest. Keratan sulfate chains modulate the biologic activity of this molecule. After the removal of the keratan sulfate chains, macrophages rapidly attach to the lumican core protein [106]. Although the state of the keratan sulfate chains in the synovial lumican molecule is unknown, this observation suggests some species of lumican could also act to localize macrophages to sites of the synovium.

Biglycan, another gene likely to be constitutively expressed by fibroblast-like intimal synoviocytes, is a dermatan sulfate-proteoglycan. It is both induced by TGF-β, and binds TGF-β [107] suggesting that biglycan may down regulate TGF-β activity by sequestering this growth factor in the extracellular matrix. IL-6 stimulates the expression of biglycan, while TNF-α depresses its expression [108].

Hyaluronan is an abundant constituent of the extracellular matrix and is especially increased in the synovial fluid. Both high and low (fragments) molecular weight forms bind to CD44. Recent studies have demonstrated that in alveolar macrophages lower molecular weight hyaluronan fragments induce the production of chemokines like IL-8 and MIP-1α through its receptor CD44, while the high molecular weight inhibits chemokine production [109]. CD44 is predominantly expressed by intimal, as opposed to subintimal, fibroblast-like synoviocytes [46, 47]. Although this pathway has not been extensively studied in fibroblast-like intimal synoviocytes, one could envision similar effects in the synovium. It is also conceivable that a similar concept may apply to other matrix components. For instance, if intact and large matrix components predominate in the synovium, representing absence of injury, a chemokine/cytokine inhibitory signal would predominate. On the other hand, when traumatic or inflammatory injury occur, signaling through hyaluronan fragments-CD44, and maybe through biglycan, lumican or other component fragments and other receptors, would activate a pro-inflammatory response to remove cellular debris, or fight an infection.

IGFBP5 (insulin-like growth factor binding protein-5) appears to be very strongly expressed by fibroblast-like intimal synoviocytes [8]. It is an important regulator of fibroblast growth that increases IGF-1 binding to the fibroblast membrane by attaching to the extracellular matrix proteins, types III and IV collagen, laminin and fibronectin [110]. IGFBP5 may have an anti-inflammatory role that opposes the effect exhibited by IL-1 and TNF-α of stimulating proteoglycan degradation and decreasing proteoglycan synthesis [111]. The observation that IGFBP5 is further induced by exposure of cells to prostaglandin E2 [112] is of interest with respect to the pattern of morphologic changes and gene activation observed in synoviocyte cultures after the addition of this agent [741.

Of some interest, a novel gene in the sulfatase family, not previously identified in any libraries, primarily prepared from non synovial sources, was identified in both fibroblast like intimal synoviocytes and subintimal synoviocytes [8]. This gene has a high degree of homology with a chondroitin sulfatase found in C. elegans and could have an interesting role in synovial matrix biology.

Other proteoglycans and glycosaminoglycans are produced by the synovial fibroblast, and the reader is referred to comprehensive book chapters or review articles [5].

The level of tyrosine phosphorylation is elevated in rheumatoid arthritis synovia compared to that found in osteoarthritis synovia, suggesting that these cells are experiencing a high degree of activation of diverse signaling pathways. These pathways are analogous to that induced by src. C-fos expression is elevated [113] and so are several other activation-related genes (see table 5). Two recently described genes that are differentially expressed in rheumatoid arthritis synovial fibroblast cultured cells are interferon-induced, and markers of cellular activation. One is the 71 kd 20-50 oligoadenylate synthetase, a subunit of one of several interferon-induced enzymes that, when activated by double-stranded RNA, converts ATP into 2′-5′ linked oligomers of adenosine [114]. The second is the interferon-inducible 56 kd protein, which has unknown function, but in common with 2Õ-5Õ oligoadenylate synthetase is strongly induced by interferons [115].

The expression of these two genes directs attention to the presence of activation-like features in the phenotype of the rheumatoid arthritis synoviocytes. Whether the expression of these genes is found in quiescent normal fibroblast-like intimal synoviocytes, whether they reflect a type of ÒmemoryÓ of being harvested from a site of immune inflammation, or whether this is a lineage specific response of fibroblast-like intimal synoviocytes to in vitro culture conditions remains to be studied. We favor the last possibility as the most likely explanation, reflecting a higher degree of responsiveness in these cells to environmental effects that could parallel their response of hyperplasia in joint injury and inflammation. Although some genes related with cell activation continue to be expressed in cultured synoviocytes others like HLA-DR appear to be more dependent on the synovial tissue pro-inflammatory environment, and become greatly reduced after 2-3 passages [116].

There are many differences between the levels of mRNA for a variety of genes that are evident between whole synovial tissues obtained from rheumatoid arthritis and osteoarthritis patients. In previous studies, a dot blot assay format using a labeled cDNA probe based on total tissue mRNA enabled parallel quantitation of the amount of message from multiple MMP and other Zn-independent protease genes. mRNA levels for stromelysin, collagenase and cathepsin D along with TIMP-1 are elevated in the representative rheumatoid arthritis sample [13, 14]. Normally, these enzymes can attack all of the elements of connective tissue, participating in histogenesis, physiologic remodeling or pathologic destruction [117]. All are synthesized as proenzymes that are activated by proteolytic cleavage. They are of particular interest to the mechanism of synovitis because firstly they are induced from very low basal levels by a variety of cytokines and growth factors but are also constitutively expressed by a variety of transformed cells. The mesenchymal cell variety of collagenase has been strongly implicated in synovitis by the finding that its mRNA is expressed at high levels in the synovial lining [118]. The identification of abundant collagenase at the protein level in the vicinity of erosions but not in equivalent abundance in other regions of the synovium suggests that it may play a special role at these sites [119]. As with other metalloproteinases, especially stromelysin, collagenases are a major product of fibroblast-like intimal synoviocytes [120] The primary action of the stromelysin is to cleave proteoglycan core and link proteins fibronectin, elastin and procollagens I, II and III, thereby mediating the remodeling of most of the matrix components other than collagen. Stromelysin also participates in collagenase activation [121]. Stromelysin mRNA is strongly expressed in rheumatoid arthritis fibroblast-like intimal synoviocytes cells [118] Ritchlin, 1991 #1214]. Immunohistochemical staining reveals that stromelysin protein to be present in fibroblasts and endothelial cells [71], as well as in monocyte lineage lining cells using in situ probing [39]. Using in situ hybridization collagenase mRNA was co-localized with that for stromelysin suggesting that the production of these two metalloproteinases is coordinated [39, 118].

The cysteine proteinase cathepsin L, which is one of the major Ras-induced proteins in Ras-transformed cells, is also identifiable in half of rheumatoid synovia, being localized to the fibroblast-like intimal cells [28]. In contrast, cathepsin B was identified in both fibroblast-like intimal synoviocytes and sub intimal synoviocytes [8].

The activated metalloproteinases bind stoichiometrically to α₂-macroglobulin in the plasma, but their major regulation after activation is through the two tissue inhibitors of metalloproteinases, TIMP-1 and TIMP-2 [122, 123]. These are two homologous molecules that are secreted in a highly regulated manner by cells elaborating metalloproteinases. The TIMPs also stoichiometrically bind to the metalloproteinases [122]. The expression of TIMP was found in the same regions where stromelysin and collagenase are expressed, but was much greater in the osteoarthritis synovial tissue compared to rheumatoid synovial tissue [39, 124]. It appears that the ratio of synthesis of TIMP to specific metalloproteinase is a critical index of the potential of a tissue to mediate matrix remodeling. In cultured synoviocytes from osteoarthritis patients there is a much higher average ratio of TIMP to stromelysin than is found in rheumatoid arthritis [125]. This suggests that fibroblast-like intimal synoviocytes are likely characterized by a higher ratio of MMP to TIMP than in sub intimal synoviocytes.

Operation of genes in the normal synovium to attract an immune response into the joint. Taken together, the phenotype of the fibroblast-like intimal synoviocyte contains an intriguing array of gene products. Some of these are shared with sub intimal synoviocytes, while others are differentially or selectively expressed in the intimal synoviocyte. Many of these gene products have the potential to be involved in normal joint histogenesis and organizing immune surveillance of the joint cavity. However, the other face of this pattern of gene expression is that these same molecules could act to foster localization of an ongoing immune response to the joint. Some of these gene products could deviate the immune response as well as intensify it. Monocytoid intimal synoviocytes could serve as antigen presenting cells with functions that might verge on those provided by dendritic cells. Moreover, this combination of cell types could provide the milieu appropriate for a form of secondary lymphoid aggregation outside the regulatory structure of the normal lymphoid organ. Thus taken together the phenotype of the lining cells could act powerfully in the afferent limb of disease development by converting an autoimmune response into an autoimmune disease.

Interestingly, the chemokine receptors expressed by T cells infiltrating the rheumatoid synovium have been described as markers for Th1 cells and na•ve T cells as well as certain dendritic cell subsets. It is conceivable that in the normal synovium similar T cells and dendritic cells would be trafficking through the joint as a part of normal immunosurveillance and remain because the lining cell environment is favorable for continued stimulation of the clone.

Additionally as discussed by Edwards, some of the genes required for germinal center formation, like VCAM-1, an important ligand for B cells, are expressed by synovial fibroblasts and may have a role in the formation of germinal centers in the inflammatory synovium. Based on these data it appears that normal fibroblast-like intimal synoviocytes can support the development of germinal centers, B cell migration and affinity maturation. For example, an additional action of SDF-1 at higher concentrations could be the facilitation of earlier stages of peripheral B-cell development in the synovial milieu that are relevant to the presence and maturation of abundant B-cells in the rheumatoid synovium and to their production of rheumatoid factors [126]. Furthermore, several additional molecules produced by the synoviocyte can interact to facilitate other aspects of B-cell development. IL-6, a cytokine with effects on B-cell differentiation, is constitutively increased in synoviocytes obtained from rheumatoid arthritis patients [14] and its synthesis by monocytes is induced by Mac-2BP, as described above. Interleukin 7-dependent proliferation of pre-B cells is also enhanced upon exposure to biglycan [127].

Hyperplasia

Hyperplasia In addition to the likely role of the fibroblast-like intimal synoviocyte in facilitating the afferent limb of the development of the autoimmune response underlying synovitis, the intima also plays a major part in the loss of function and joint destruction that characterize fully developed rheumatoid arthritis. A feature of the rheumatoid synovium is the marked hyperplasia of the lining layer and the apparent invasion and destruction of cartilage and other joint structures by the mesenchymally-derived fibroblasts and the bone marrow derived monocytoid lineage cells. The cell biology of this response is covered in two chapters (apoptosis and cartilage destruction). The changes in the lining during hyperplasia include a massive increase in the number of fibroblast-like intimal synoviocytes and an altered cell-cell relationship with the monocytoid lineage synoviocytes. In fact, in a parallel to synovial hyperplasia there is also loss of contact inhibition of rheumatoid arthritis cultured synovial fibroblasts with a disorganized accumulation of cells [4, 20, 24]. We interpret this as a reflection of the normal biology of the fibroblast-like intimal synoviocyte revealed by its response to culture conditions.

In rheumatoid arthritis, it is unknown whether initiation of the autoimmune response and its localization to the joint occurs in the setting of entirely normal intima, or whether minor degrees of non-specific hyperplasia could play a role in localizing an immune response into the joint through the repertoire of immunologically relevant molecules expressed by these cells. Hyperplasia could be initiated by a non-specific minor traumatic event or even driven by a local immune response to a common pathogen, and the constitutive production of such chemokines might provide a non antigen-specific mechanism for localizing potential pathogenic immune responses to the joint. In other words, the production of such chemoattractant molecules would be part of the normal function of the fibroblast-like intimal synoviocytes and would have increased transcription when either activated or subjected to an inflammatory imprinting, or when the number of cells increase. The unusual behavior of fibroblast-like intimal synoviocytes in culture may reflect this behavior.

Hyperplasia appears to be an intrinsic response of intimal synoviocytes to injury and healing. Apparently in response to the events initiated within the T-cell compartment of the joint tissues, the synovial membrane undergoes this striking change in its form and in its pattern of gene expression. It is transformed from a nutritive tissue into one that is the central agent of joint destruction, most notably focussed on causing injury to the cartilage through expression of enhanced levels of degradative enzymes and through secretion of cytokines that can act to alter the pattern of gene expression in the chondrocyte. This alteration in the synovium involves a massive influx of monocyte-lineage cells and extensive neovascularization as well as marked hyperplasia of the intimal synoviocytes, likely mediated, in part, by genes described above. Three sets of biologic events are evident. 1. The intrinsic biology of the fibroblast-like intimal synoviocyte, where increased cell number simply is reflected as increased local concentration of mediators and cell surface molecules, 2. The pathways of mutual interaction of fibroblast-like and monocytoid intimal synoviocytes, and 3. Paracrine influences of the products of the autoimmune response on the intimal synoviocytes. It is possible that the loss of normal cell-matrix signals due to hyperplasia and its replacement by more extensive cell-cell receptor interactions result in reverse signaling that leads to a perpetuation of the hyperplasia.

However, the question remains as to why is it that this physiologic process performed by the synovial fibroblasts gets out of control leading to massive cell proliferation and invasion of cartilage? Although a definitive response for this question is not presently at hand, it has been recently considered that these cells not only are activated and have an increased proliferation rate, but also their cellular turnover through apoptosis is decreased, further-contributing to the cellular accumulation and hyperplasia seen in rheumatoid arthritis fibroblast-like intimal synoviocytes. This could well be a feature of cells of the synoviocyte lineage. Several genes overexpressed in these cells and involved in the increased cytokine expression as well as in the regulation of cell proliferation and/or survival in the rheumatoid synovium provide clues to the regulation of hyperplasia. Among these genes, recent studies suggest that NF-κB has a key role in the regulation of synovial fibroblast apoptosis and gene expression (discussed below). NF-κB is highly expressed in the rheumatoid synovium and synovial fibroblasts [128], and its inhibition has been demonstrated to render these cells more susceptible to both TNF-α and FAS-L mediated apoptosis [129]. Furthermore, its inhibition greatly decreases IL-1, IL-6, TNF-α and VCAM-1 expression in both Streptococcal cell wall- and pristane-induced arthritis in rats, thus not only regulating local and systemic mediators of joint injury, but also decreasing the expression of critical molecules involved in homing of lymphocytes to the joint [129].

Proliferation and Increased Oncogene Expression

Whether these events found in hyperplastic synoviocytes in rheumatoid arthritis are specific, unique changes that lead to the joint destruction in this disease, or whether they are simply a reflection of the intrinsic hyperresponsiveness of these cells to signalling circuits remains to be established. Fibroblast-like intimal synoviocytes from rheumatoid arthritis and other inflammatory arthropaties survive and continue to proliferate after several passages in culture. These cells have increased expression of oncogenes and proteins involved in cell-cycle regulation, mitosis and production of growth factors and cytokines. This is a reflection of the intrinsic property of the fibroblast-like intimal synoviocytes. Increased expression of some of these molecules, including a number of oncogenes, mediate cell proliferation and hyperplasia. Both autocrine and paracrine factors are involved in the regulation of these genes. Among the genes overexpressed in rheumatoid arthritis synovium and cultured synovial fibroblasts is egr-1 [131, 132] which regulates the transcription of ras and sis and is down-regulated by p53. c-fos and c-jun regulate the transcription of IL-1, IL-6, TNF and MMPs, and both oncogenes have their expression increased in rheumatoid arthritis fibroblast-like synoviocytes [113, 131, 133]. In fact, inhibition of c-fos reduced synovial fibroblast proliferation in culture [134], and ameliorated collagen-induced arthritis in mice [135]. C-myc [13, 28], like ras [28], is sometimes highly expressed in fibroblast-like intimal synoviocytes. Ras is involved in the regulation of cathepsin L expression, a protease involved in cartilage degradation [28]. Additionally, H-ras point mutations of yet unknown significance have been recently described in rheumatoid arthritis and osteoarthritis synovium [136]. Other oncogenes and proteins involved in cellular proliferation have been identified in rheumatoid arthritis synovial fibroblasts including PCNA, NOR [137], c-sis/PDGF [138]. The question remains whether the primary defect is an abnormal proliferation that must occur through the well-known pathways involving oncogenes, or alternatively, if the fundamental defect in rheumatoid arthritis is altered oncogene expression with increased proliferation being a consequence.

The expression of oncogenes and their down-stream regulatory functions in cellular replication and production of proteolytic enzymes like MMP, cathepsin and others is critical in the cartilage and bone damage caused by the infiltrating fibroblast-like intimal synoviocytes. Additionally, these genes interact with several genes and gene products regulating apoptosis, however, little of those interactions have been studied in the rheumatoid arthritis synovium.

Anormalities in Synovial Apoptosis

It has been suspected that not only the fibroblast-like intimal synoviocytes proliferation is increased, but also its longevity is increased, possibly due to defects in the regulation of apopotosis. Independent groups described abnormalities in apoptosis of rheumatoid arthritis fibroblast-like intimal synoviocytes, with increased number of apoptotic figures along the lining layer [139, 140]. Although increased numbers of apoptotic figures are seen in the fibroblast-like intimal synoviocytes, it is probably still an insufficient rate proportionally to the high rate of proliferation of those cells. The inbalance between proliferation and cell death, would lead to an increased accumulation of these cells [139, 140]. Increased Fas expression was identified in the rheumatoid fibroblast-like intimal synoviocytes [139-141]. Not only Fas was expressed, but also apoptosis could be induced with anti-Fas antibodies, demonstrating that Fas-mediated apoptosis pathway was preserved in these cells [139]. Subsequently, animal studies demonstrated that anti-Fas antibodies significanty ameliorate arthritis in mice [142, 143] raising the possibility of using pro-apoptotic strategies to eliminate the proliferating fibroblast-like intimal synoviocytes. However, despite the apparent integrity of the Fas-mediated pathway in rheumatoid fibroblast-like intimal synoviocytes, it was recently demonstrated that several pro-inflammatory molecules abundant in the rheumatoid synovium, like IL-1, IL-6, IL-8, TNFα, [144], and TGF-β [145]1, are capable of down-regulating Fas expression, thereby potentially preventing Fas-mediated apoptosis in vivo, and contributing to increased accumulation of the proliferating cells.

Other genes involved in cellular proliferation and apoptosis have also been studied. Among those, p53 expression was increased in rheumatoid arthritis fibroblast-like intimal synoviocytes by immunohistochemistry in synovial tissue and in cultured cells [146]. Firestein et al hypothesized that the increased p53 expression could be secondary to increased numbers of somatic mutations in the synovial tissue caused by local injury, for example due to oxygen radicals, and possibly even mutations on p53 leading to an inefficient induction of apoptosis. These investigators identified somatic mutations on the p53 gene in 7 out of 15 rheumatoid arthritis patients synovial fibroblasts [147]. The same group subsequently demonstrated that inhibiting p53 function in rheumatoid arthritis and normal fibroblast-like synoviocytes could change cellular survival, susceptibility to apoptosis and cartilage invasiveness [148]. However, it is not clear how often those mutations occur and whether a mutation rate of 1 to 100 or 1 to 1000 synovial fibroblasts would still be biologically relevant. Despite the presence of p53 mutations, cancer does not develop in the rheumatoid arthritis synovium, perhaps due to the absence of other mutated genes required for cancer development, or maybe due to some unknown synovial protective factor [147]. Semaphorin may also play a role in view of its identity as a multidrug resistance element and possible role in tumorgenesis. [97, 98]

An alternative mechanism of regulation of apoptosis is through TNF-mediated pathways. TNF-α is abundant in the joint, and activation of its receptors leads to increase levels of NF-κB, a transcription factor that is involved in the regulation of cellular proliferation which has an anti-apoptotic effect [149]. NF-κB is highly expressed in rheumatoid arthritis fibroblast-like synoviocytes and inhibition of its activity with N-acetyl-L-cysteine dramatically reduced the rate of cellular proliferation in vitro [128]. Recently, Miagkov et al studying Streptococcal cell wall and pristane-induced arthritis in rats demonstrated that NF-κB is expressed early on during the development of arthritis, and that the inhibition of NF-κB rendered synovial fibroblasts susceptible to TNF and FAS-mediated apoptosis [129]. Interestingly enough, some of the drugs used to treat RA, like gold salts and glucocorticoids, have also been demonstrated to interfere with the NF-κB activity [150-152], although it is not known how much of the effects of these drugs is due to an action on fibroblast-like intimal synoviocytes.

Conflicting findings have been described regarding the expression of the anti-apoptosis gene Bcl-2 in rheumatoid arthritis synovial fibroblasts [140, 153, 154] However, its expression appears to be upregulated by the same pro-inflammatory molecules present in the rheumatoid synovium that down-regulate Fas expression, thereby favoring an anti-apoptosis, pro-survival stimuli [144].

Strategies aiming at modifying the rheumatoid fibroblast-like intimal synoviocytes cell turn-over, either by increasing fibroblast-like intimal synoviocytes apoptosis or decreasing the cellular proliferation rate may prove helpful in managing RA, and the identification of such genes, and the better understanding of their function should lead to the development of new therapeutic agents.

Cartilage Injury

The ultimate clinically relevant consequences of joint inflammation in rheumatoid arthritis are the pain, tenderness and loss of function of synovitis and the destruction of cartilage mediated by the synovial events. Cartilage injury likely proceeds by two distinct mechanisms. An indirect one in which cytokines released by the synovial lining cells and infiltrating mononuclear cells activate chondrocytes to a pattern of gene expression that results in remodeling and degradation of the cartilage matrix. For example, IL-1 stimulates chondrocytes to release degradative enzymes [155, 156] and a direct mechanism in which metalloproteinases and other enzymes released by the fibroblast-like intimal synovicytes and perhaps the infiltrating monocytes directly act to digest the matrix [157, 158]. The junction between the hyperplastic synovium and the cartilage appears to be the principal site of interaction between these former biologic allies and members of the same lineage. Assessment of the rate and character of cartilage injury has been determined by measuring the fine structure of the products of proteoglycan fragmentation. The glycosaminoglycan rich region of the core protein predominates during the early phase of cartilage injury before there is significant damage evident on conventional radiographs [159]. Later when frank radiographic changes are evident, the joint fluid contains an abundance of hyaluronan binding domains and lesser amounts of the glycosaminoglycan rich region of the core protein. The fact that the disruption of the stromelysin-1 (MMP-3) gene did not protect mice from developing cartilage destruction in CIA suggests that redundant or compensatory functions exist among MMPs or between MMPs and other genes [160].

Recent studies have demonstrated that some types of cartilage injury occur without the presence of T cells, likely reflecting the constitutive release of cytokines, such as IL-1, described above. In experimental models where rheumatoid arthritis synovial fibroblasts were implanted together with cartilage in SCID mice, cartilage erosions occurred despite the absence of T cells [161, 162], suggesting that fibroblast like intimal synoviocytes have the intrinsicpotential to mediate this matrix remodeling. Additionally, H2-c-fos transgenic mice develop chronic arthritis, and the synovial proliferation and joint erosion occur in the absence of infiltrating lymphocytes in the synovium [163]. Scott et al [164] also demonstrated that the cartilage degradation depended on a critical fibroblast-like intimal-synoviocytes-macrophage interaction which was IL-1, IL-6, TNFα and CD44 dependent.

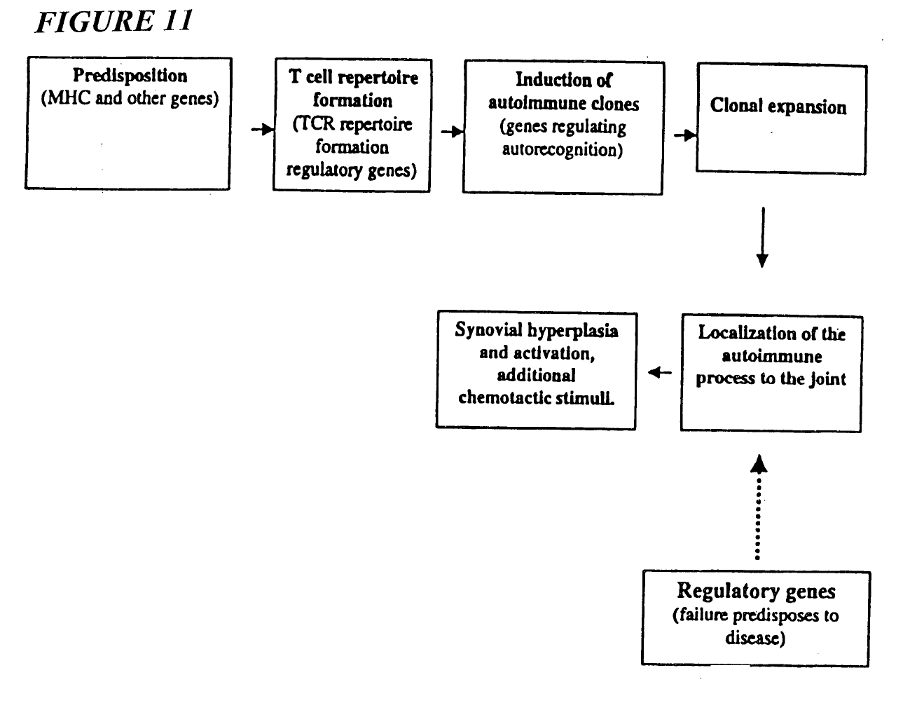

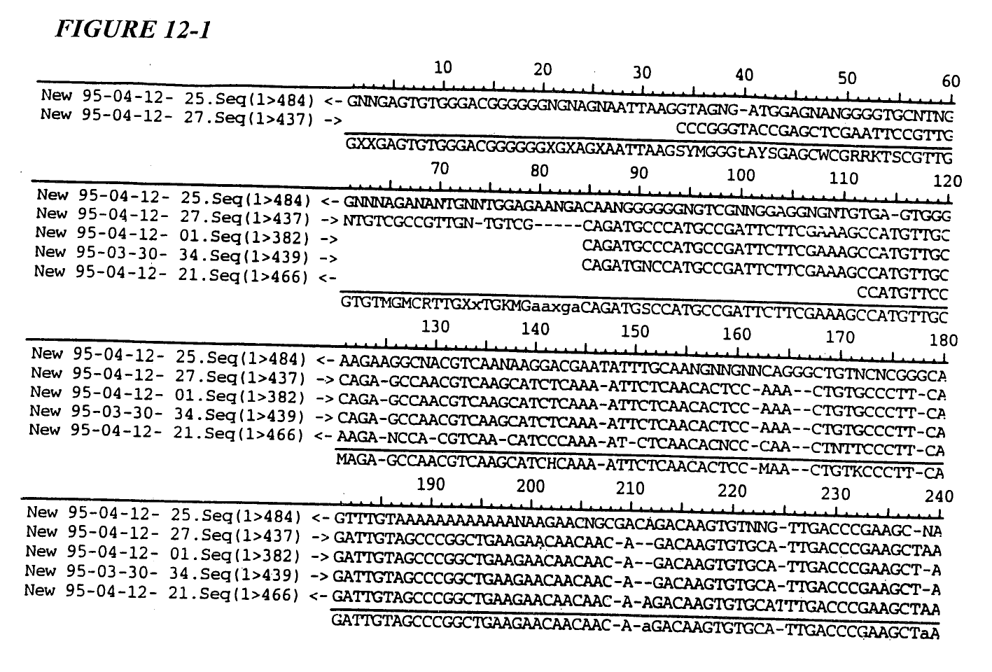

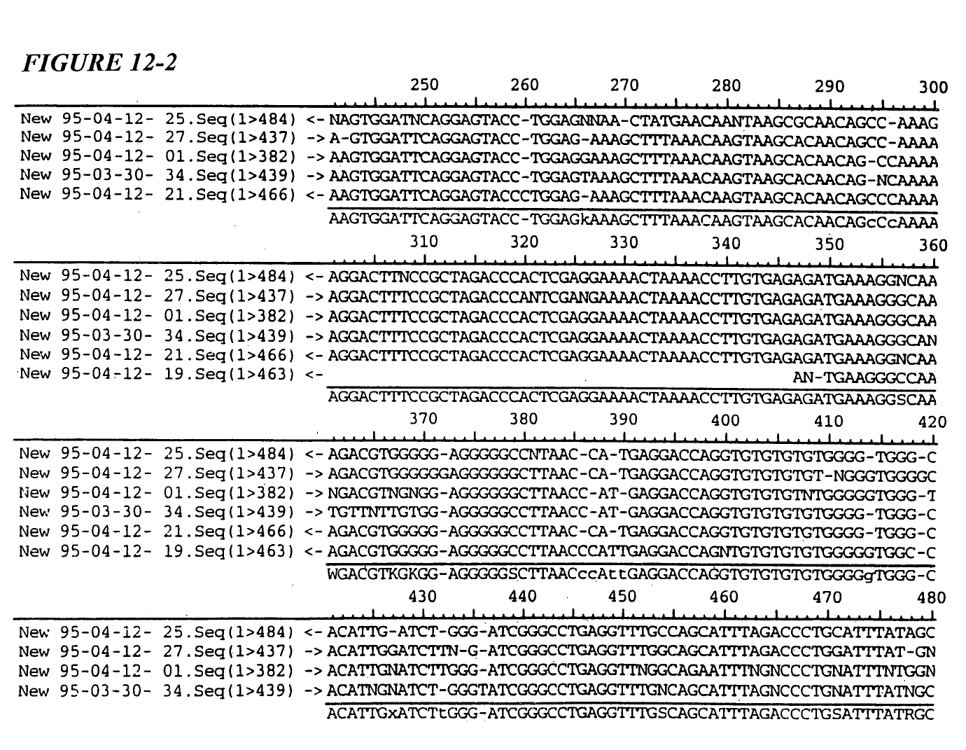

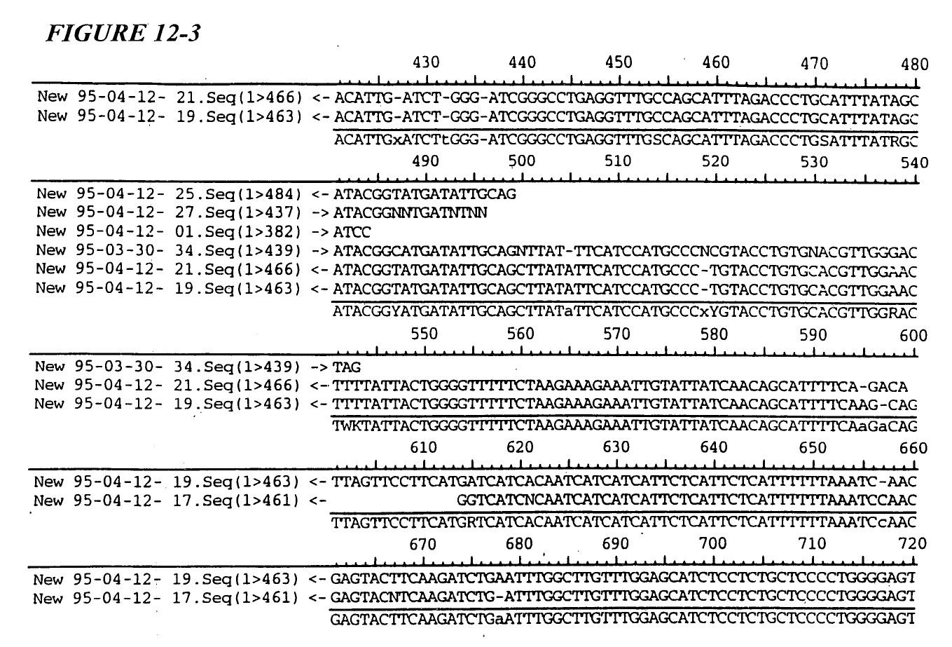

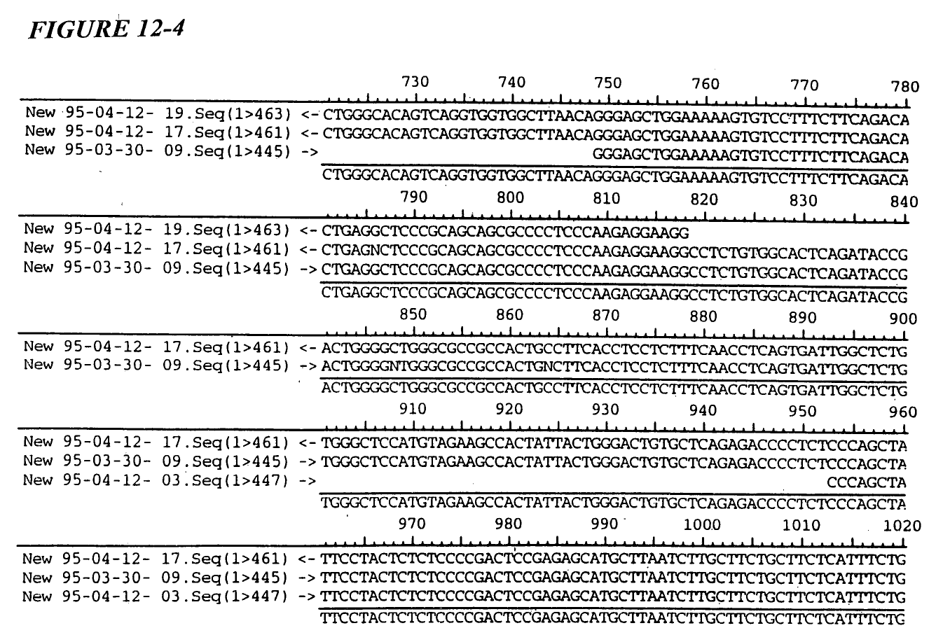

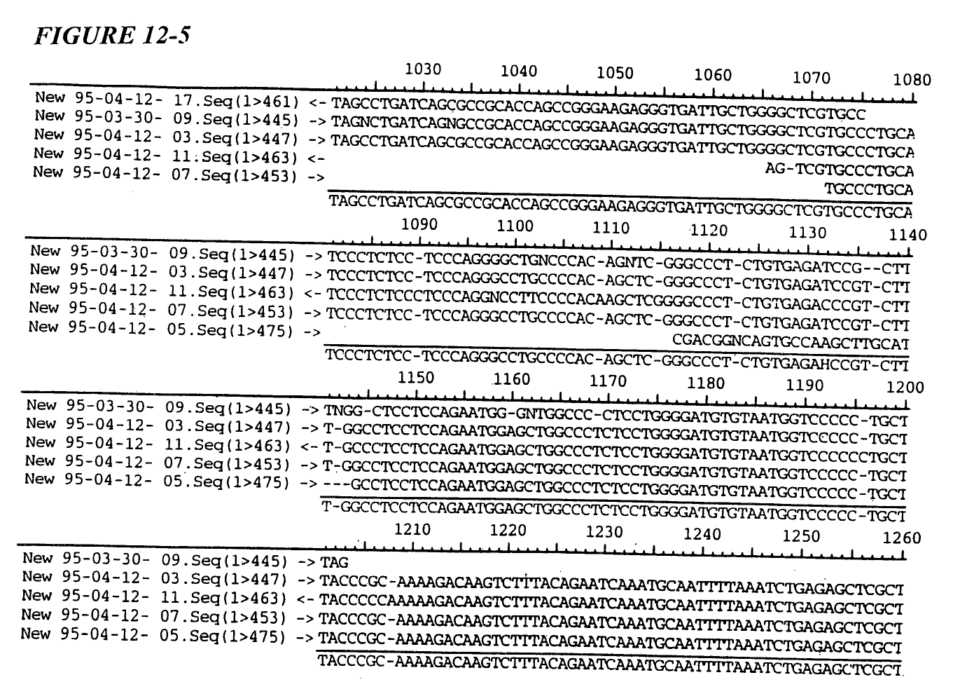

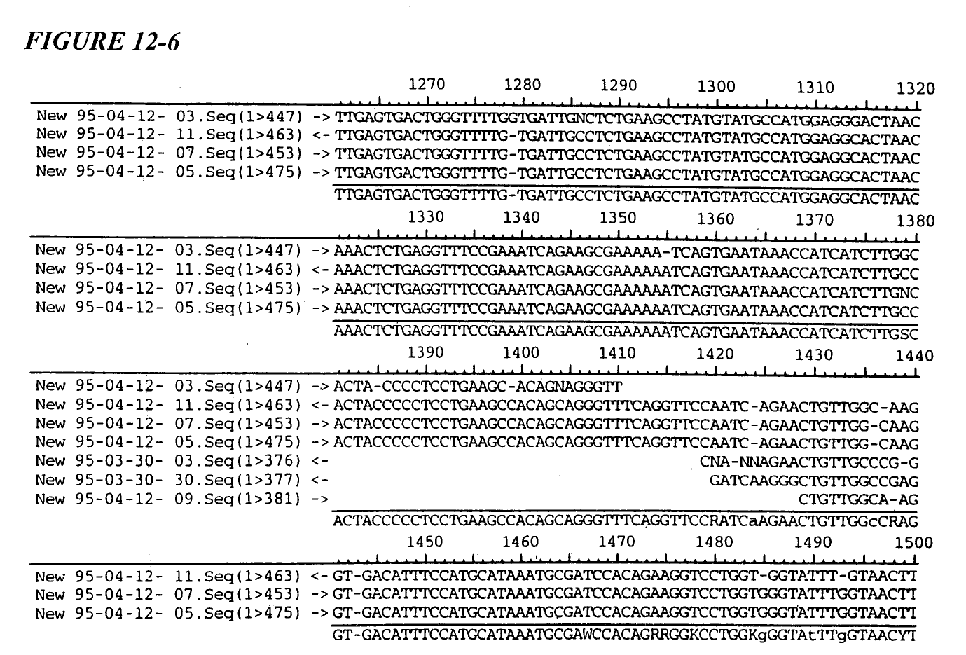

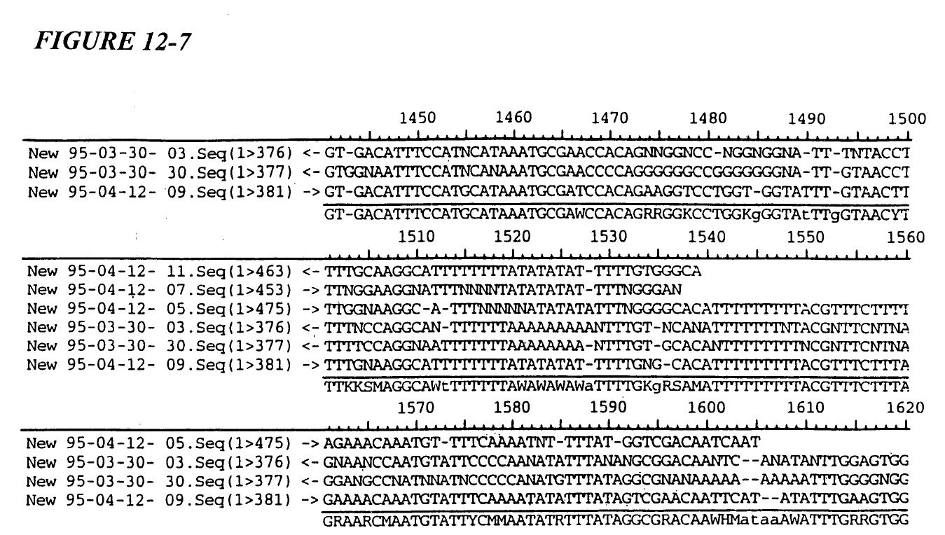

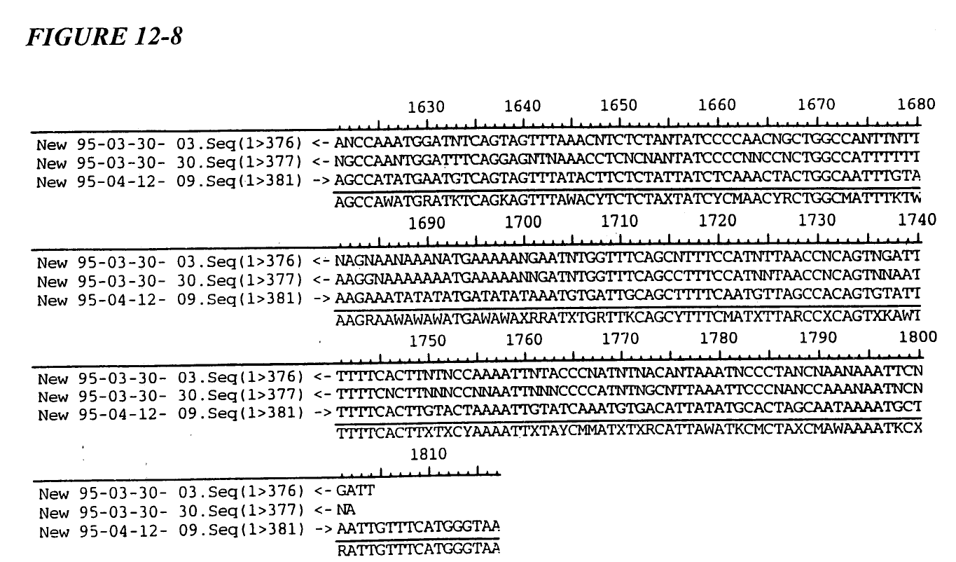

It is very likely that the development of rheumatoid arthritis proceeds through a variety of stages from susceptibility through the development of autoreactive T cell clones to overt disease as shown in FIG. 11. In particular macrophages and fibroblast-like intimal synoviocytes may have a more important role in localizing the autoimmune process to joint and in its perpetuation, as discussed earlier.

Genes regulating cellular functions at each one of these stages of disease development could be candidate susceptibility/severity genes and potential targets for therapy. One approach for additional gene discovery efforts could be the use of similar strategies used by our group [8, 12, 13], or using hybridization membranes, SAGE [165] or cDNA/EST hybridization microarrays [166] to study fibroblast-like intimal synoviocytes gene expression in very early RA, or to study in a similar fashion macrophages or T cells derived from the synovium and compare gene expression patterns with normal, osteoarthritis or tissues obtained from patients with established RA. This kind of approach would shed additional light regarding the contribution of different genes products at each stage of disease. In fact, a similar strategy could be used to determine which genes down or up regulation are critical for clinical improvement and response to drug therapy. It was first postulated by Dayer et al [167] that the lymphocytic response which initiates the cascade of immune interactions and cytokine production by acting directly on the target fibroblast cells and indirectly on them by activating monocyte lineage cells to release additional cytokines. Thus T cells may be more involved in the early stages of disease, while in chronic stages the inflammatory drive would be more macrophage and fibroblast-like intimal synoviocytes-dependent. [35, 168].

Genomic Dissection of the Fibroblast-Like Intimal Synoviocytes Phenotype

Some of the genes previously discussed may differentially regulate cellular functions at each one of these stages of disease development, they are assuming increasing importance as non-MHC genes involved in the definition of susceptibility This is especially so in the context of the paradigm underlying this chapter that distinguishes between an autoimmune response and the localization of the response to the joint that results in disease. Linkage studies have been done in both rheumatoid arthritis and in experimental models of arthritis in rodents to identify novel non-MHC genes. This type of analyses try to identify co-segregation between phenotype and genotype without prior knowledge of the trait causing/regulating genes. Linkage analysis is a powerful tool to identify new genes and new pathways involved in the regulation of a particular phenotype. Several susceptibility loci have been identified both in rodents and humans. It is not known which genes account for those susceptibility loci, however, several of them map to genomic regions containing some of the genes discussed herein (Table 4). Therefore, some of these genes are candidate susceptibility genes. In fact, some may be involved in the regulation of disease severity as well [89, 169-171] (Dracheva et al unpublished observations). How much of this differential gene expression is regulated at the germline genetic level versus being determined at the somatic level is unknown.

Among those candidate genes, one of the genes differentially expressed (biglycan) is located in genomic intervals where a rheumatoid arthritis susceptibility locus has been mapped [172] (Table 4). Additionally, 4 other differentially expressed genes (Semaphorin, VCAM-1, Lumican, Interferon-induced 71 kd 2050 oligoadenylate synthetase) map to human chromosomal intervals syntenic with rat regions where loci regulating experimental animal erosive arthritis have been mapped [89, 170-172], (Dracheva et al, unpublished observations).

For linkage analysis it is critical to have a well-defined phenotype to be mapped. Rheumatoid arthritis is a heterogeneous disease with manifestations that may vary from one patient to the other, creating potential confusing factors. Different clinical and laboratory manifestations may be regulated by different genes. One approach that may facilitate the genetic dissection of rheumatoid arthritis is to study sub-phenotypes of the disease, like for example, whether allelic genes are responsible for particular elements in the distinctive fibroblast-like intimal synoviocyte phenotype. For example, a certain gene may be more important to the capacity of a fibroblast-like intimal synoviocytes to degrade cartilage than it is to the complete phenotype that is RA. By sub-phenotyping disease we may obtain more clean phenotypes and increased the likelihood of identifying linkage.

### CONCLUSION

The synovial fibroblast, particularly the intimal cells, have a central role in localizing the autoimmune response in rheumatoid arthritis to the joint, and further, that this role in the afferent arm of the development of autoimmune disease may be in part an extension of the normal function of these stem-like cells seen during development, embryogenesis and in normal synovial physiology. It was also proposed that part of the differential gene expression seen in cultured rheumatoid arthritis synovial fibroblast is lineage-dependent and related to the initial proportion of intimal mesenchymal stem-like to subintimal cells in the biopsy or surgical specimens, with the differential expression representing increased number, or hyperplasia, of intimal cells. Thus much of the distinctive phenotype of cultured rheumatoid arthritis synoviocytes could be a combination of the intrinsic pattern of gene expression in this stem cell-like sublineage and its pattern of response to culture in vitro. The pattern of gene expression seen in the fibroblast-like synoviocyte suggests that this cell may represent a form of less differentiated cell, closer to the mesenchymal stem-cell than to the typical fibroblast.

The synovial fibroblast gene products operate in an autocrine and paracrine pattern further favoring the programmed constitutive functions of this intriguing cell. However, it is possible that among what we are considering as lineage differences there may be critical allelic differences governing gene expression that confer enhanced susceptibility for the development of rheumatoid arthritis. This possibility needs to be tested. Abnormalities in the expression of genes involved in the regulation of cell proliferation, like oncogenes, transcription factors critical for cytokine production and regulation of apoptosis, as well as other apoptosis regulatory genes, cytokines and chemokines could be a factor fostering either the afferent function of fibroblast-like intimal synoviocytes or their efferent effector function. Part of these abnormalities may relate to somatic mutations in the synovium, and part of this effect may be under germline genetic regulation. It was proposed that some of the genes regulating the fibroblast-like intimal synoviocyte, in the presence of other genes required in the earlier stages in the development of autoimmunity, would culminate in disease. In fact, several of the genes differentially expressed in rheumatoid arthritis as compared to osteoarthritis fibroblast-like synoviocytes in culture are located in chromosomal regions previously described to contain arthritis susceptibility and severity regulatory genes.

The combined study of gene expression, perhaps by cDNA microarray technologies, and function in synovial fibroblasts and linkage analysis may facilitate the gene discovery efforts in rheumatoid arthritis by creating simplified phenotypes, and the identification of these regulatory genes is likely to provide new targets for therapy as well as increase our understanding of the pathogenesis of arthritis.

### REFERENCES

Winchester R Rheumatoid Arthritis. Edited by M M Frank, K F Austen, H N Claman, E R Unanue. Sampter's Immunological Diseases: Boston, Mass.: Little, Brown and Company; 1995.
Barland P, Novikoff A B, Hamerman D Electron microscopy of the human synovial membrane. J Cell Biol 1962; 14:207-220.
Norton W L, Ziff M Electron microscopic observations in the rheumatoid synovial membrane. Arthritis Rheum 1966; 9:589-610.
Burmester G R, Dimitriu-Bona A, Waters S J, Wincnester R J Identification of three major synovial lining cell populations by monoclonal antibodies directed to Ia antigens and antigens associated with monocytes/macrophages and fibroblasts. Scand J Immunol 1983; 17:69-82.
Edwards J C, Leigh R D, Cambridge G Expression of molecules involved in B lymphocyte survival and differentiation by synovial fibroblasts. Clin Exp Immunol 1997; 108:407-414.
Morales-Ducret J, Wayner E, Elices M J, Alvaro-Gracia J M, Zvaifler N J, Firestein G S Alpha 4/beta 1 integrin (VLA4) ligands in arthritis. Vascular cell adhesion molecule-1 expression in synovium and on fibroblast-like synoviocytes. J Immunol 1992; 149:1424-1431.
Klareskog L, Forsum U, Scheynius A, Kabelitz D, Wigzell H Evidence in support of a self-perpetuating HLA-DR-dependent delayed-type cell reaction in rheumatoid arthritis. Proc Natl Acad Sci USA 1982; 79:3632-3636.
Trabandt A, Aicher W K, Gay R E, Sukhatme V P, Nilson Hamilton M, Hamilton R T, et al Expression of the collagenolytic and Ras-induced cysteine proteinase cathepsin L and proliferation-associated oncogenes in synovial cells of MRL/I mice and patients with rheumatoid arthritis. Matrix 1990; 10:349-361.
Firestein G S, Zvaifler N J How important are T cells in chronic rheumatoid synovitis? Arthritis Rheum 1990; 33:768-73.
Arend W P, Dayer J M Cytokines and cytokine inhibitors or antagonists in rheumatoid arthritis. Arthritis Rheum 1990; 33:305-15.
Koch A E, Kunkel S L, Burrows J C, Evanoff H L, Haines G K, Pope R M, et al. Synovial tissue macrophage as a source of the chemotactic cytokine IL-8. J Immunol 1991; 147:2187-2195.
Winchester R J, Burmester G R Demonstration of Ia antigens on certain dendritic cells and on a novel elongate cell found in human synovial tissue. Scand J Immunol 1981; 14:439-444.
Werb Z, Mainardi C L, Vater C A, Harris E D, Jr. Endogenous activation of latent collagenase by rheumatoid synovial cells. N Engl J Med 1977; 296:1017.
Castor C W, Ritchie J C, Scott M E, Whitney S L Connective tissue activation X1: stimulation of glycosaminoglycan and DNA formation by a platelet factor. Arthritis Rheum 1977; 20:859-868.
Bucala R, Ritchlin C, Winchester R, Cerami A Constitutive production of inflammatory and mitogenic cytokines by rheumatoid synovial fibroblasts. J Exp Med 1991; 173(3):569-574.
Smith C A Properties of synovial cells In culture. J Exp Med 1971; 134:306S.
Wynne-Roberts C R, Castor C W Ultrastructural comparison of rheumatoid and nonrheumatoid synovial fibroblasts grown in tissue culture. Arthritis Rheum 1972; 15:65-83.
Anastassiades T P, Len J, Wood A, Irwin D The growth kinetics of synovial fibroblastic cells from inflammatory and noninflammatory arthropathies. Arthritis Rheum 1978; 21:461-466.
Ponteziere C, Desmoulins D, Agneray J, Ekindjian O G, Cals M J Comparative proliferation of non-rheumatoid and rheumatoid human synovial cells. Int J Tissue React 1990; 12:229-236.
Goddard D H, Grossman S L, Moore M E Autocrine regulation of rheumatoid arthritis synovial cell growth in vitro Cytokine 1990; 2:149-155.
Winchester R, Su F, Ritchlin C Modulation of synoviocytes by inflammation—source of a persistent non-immunologic drive in synovitis: analysis of levels of mRNA expression by a simple multi-gene assay. Clin Exp Rheumatol 1993; 11(S8):87-90.
Kriegsmann J, Keyszer G M, Geiler T, Brauer R, Gay R E, Gay S Expression of vascular cell adhesion molecule-1 mRNA and protein in rheumatoid synovium demonstrated by in situ hybridization and immunohistochemistry. Lab Invest 1995; 72:209-214.
Ritchlin C T, Winchester R J Potential mechanisms for coordinate gene activation in the rheumatoid synoviocyte: implications and hypotheses. Springer Semin Immunopathol 1989; 11:219-234.
Ritchlin C, Dwyer E, Bucala R, Winchester R Sustained and distinctive patterens of gene activation in synovial fibroblasts and whole synovial tissue obtained from inflammatory synovitis. Scand J Immunol 1994; 40:292-8.
Lisitsyn N, Wigler M Cloning the differences between two complex genomes. Science 1993; 259:946-951.
Hubank M, Schatz D G Identifying differences in mRNA expression by representational difference analysis of cDNA. Nucleic Acids Res 1994; 22:5640-5648.
Sambrook J, Fritsch E F, Maniatis T: Molecular Cloning A laboratory Manual. 2nd edition. Cold Spring Harbor, N.Y., Cold Spring Harbor Laboratory, 1989
Seki T Identification of multiple isoforms of the low-affinity human IgG Fc receptor. Immunogenetics 1989; 30:5-12.
Shirozu M, Nakano T, Inazawa J, Tashiro K, Tada H, Shinohara T, et al Structure and chromosomal localization of the human stromal cell-derived factor 1 (SDF1) gene. Genomics 1995; 28:495-500.
Tan P L, Farmiloe S, Yeoman S, Watson J D Expression of the interleukin 6 gene in rheumatoid synovial fibroblasts. J Rheumatol 1990; 17:1608-1612.
Koths K, Taylor E, Halenbeck R, Casipit C, Wang A Cloning and characterization of a human Mac-2-binding protein, a new member of the superfamily defined by the macrophage scavenger receptor cysteine-rich domain. J Biol Chem 1993; 268:14245-14249.
Inohara H, Akahani S, Koths K, Raz A Interaction between galectin-3 and Mac-2-binding protein mediate cell-cell adhesion. Cancer Res 1996; 56:4530-4534.
Yu B, Wright S D LPS-dependent interaction of Mac-2-binding protein with immobilized CD14. J Inflamm 1995; 45:115-125.
Iacobelli S, Arno E, D'Orazio A, Coletti G Detection of antigen recognized by a novel monoclonal antibody in tissue and serum from patients with breast cancer. Cancer Res 1986; 46:3005-3010.
Ullrich A, Sures I, D'Egidio M, Jallal B, Powell T J, Herbst R, et al The secreted tumor-associated antigen 90K is a potent immune stimulator. J Biol Chem 1994; 269:18401-18407.
Luo Y, Shepherd I, Li J, Renzi M J, Chang S, Raper J A A family of molecules related to collapsin in the embryonic chick nervous system. Neuron 1995; 14:1131-1140.
Hall K T, Boumsell L, Schultze J L, Boussiotis V A, Dorfman D M, Cardoso A A, et al Human CD100, a novel leukocyte semaphorin that promotes B-cell aggregation and differentiation. Proc Natl Acad Sci USA 1996; 93:11780-5.
Mangasser-Stephan K, Dooley S, Welter C, Mutschler W, Hanselmann R G Identification of human semaphorin E gene expression on rheumatoid synovial cells by mRNA differential display. Biochem Biophys Res Commun 1997; 234:153-156.
Nagasawa T, Kikutani H, Kishimoto T Molecular cloning and structure of a pre-B-cell growth-stimulating factor. Proc Natl Acad Sci USA 1994; 91:2305-2309.
Tashiro K, Tada H, Heilker R, Shirozu M, Nakano T, Honjo T Signal sequence trap: a cloning strategy for secreted proteins and type I membrane proteins. Science 1993; 261:600-603.
D'Apuzzo M, Rolink A, Loetscher M, Hoxie J A, Clark-Lewis I, Melchers. F, et al The chemokine SDF-1, stromal cell-derived factor 1, attracts early stage B cell precursors via the chemokine receptor CXCR4. Eur J Immunol 1997; 27:1788-1793.
Ajuti A, Webb I J, Bleul C, Springer T, Gutierrez—Ramos J C The chemokine SDF-1 is a chemoattractant for human CD34+ hematopoietic progenitor cells and provides a new mechanism to explain the mobilization of CD34+ progenitors to peripheral blood. J Exp Med 1997; 185:111-120.
Nagasawa T, Hirota S, Tachibana K, Takakura N, Nishikawa S, Kitamura Y, et al Defects of B-cell lymphopoiesis and bone-marrow myelopoiesis in mice lacking the CXC chemokine PBSF/SDF-1. Nature 1996; 382:635-8.
Bleul C C, Farzan M, Choe H, Parolin C, Clark-Lewis I, Sodroski J, et al The lymphocyte chemoattractant SDF-1 is a ligand for LESTR/fusin and blocks HIV-1 entry. Nature 1996; 382:829-32.
Bleul C C, Fuhlbrigge R C, Casasnovas J M, Aiuti A, Springer T A A highly efficacious lymphocyte chemoattractant, stromal cell-derived factor 1 (SDF-1). J Exp Med 1996; 184:1101-9.
Rada J A, Cornuet P K, Hassell J R Regulation of corneal collagen fibrillogenesis in vitro by corneal keratan sulfate proteoglycan (lumican) and decorin core proteins. Exp Eye Res 1993; 56:635-48.
Grover J, Chen Z N, Korenberg J R, Roughley P J The human lumican gene. Organization, chromosomal location, and expression in articular cartilage. J Biol Chem 1995; 270:21942-21949.
Funderburgh J L, Mitchler R R, Funderburgh M L, Roth M R, Chapes S K, Conrad G W Macrophage receptors for lumican. Invest Ophthalmol Vis Sci 1997; 38:1159-1167.
Hildebrand A, Romaris M, Rasmussen L M, Heinegard D, Twardzik D R, Border W A, et al Interaction of the small interstitial proteoglycans biglycan, decorin and fibromodulin with transforming growth factor beta. Biochem J 1994; 302:527-534.
Ungefroren H, Krull N B Transcriptional regulation of the human biglycan gene. J Biol Chem 1996; 271:15787-15795.
Jones J I, Gockerman A, Busby W H, Jr, Camacho-Hubner C, Clemmons D R Extracellular matrix contains insulin-like growth factor binding protein-S: potentiation of the effects of IGF-I. J Cell Biol 1993; 121:679-687.
Tyler J A Insulin-like growth factor 1 can decrease degradation and promote synthesis of proteoglycan in cartilage exposed to cytokines. Biochem J 1989; 260:543-548.
Pash J M, Canalis E Transcriptional regulation of insulin-like growth factor-binding protein-5 by prostaglandin E2 in osteoblast cells. Endocrinology 1996; 137:2375-2382.
Krane S M, Dayer J M, Simon L S, Byrne M S Mononuclear cell-conditioned medium containing mononuclear cell factor (MCF), homologous with interleukin 1, stimulates collagen and fibronectin synthesis by adherent rheumatoid synovial cells: effects of prostaglandin E2 and indomethacin. Coll-Relat-Res 1985; 5:99-117.
Marie I, Hovanessian A G The 69-kDa 2-5A synthetase is composed of two homologous and adjacent functional domains. J Biol Chem 1992; 267:9933-9939.
Wathelet M, Moutschen S, Defilippi P, Cravador A, Collet M, Huez G, et al Molecular cloning, full-length sequence and preliminary characterization of a 56-kDa protein induced by human interferons. Eur J Biochem 1986; 155:11-17.
Mellors R C, et. al. Rheumatoid factor and the pathogenesis of rheumatoid arthritis. J Exp Med 1961; 113:475.
Oritani K, Kincade P W Identification of stromal cell products that intereact with pre-B cells. J Cell Biol 1996; 134:771-782.
Grimley P M, Sokoloff L Synovial giant cells rheumatoid arthritis. Am J Pathol 1966; 49:931.
Rankin, E. C. et al. The therapeutic effects of an engineered human anti-tumour necrosis factor α antibody (CDP 571) in rheumatoid arthritis. Br J Rheumatol 1995 April 34(4):334-42.
Dinant, H. J. and Dijkmans, B. A. New therapeutic targets for rheumatoid arthritis. Pharm World Sci 1999 April 21(2):49-59.
Maini, R. N. et al. Therapeutic efficacy of multiple intravenous infusions of anti-tumor necrosis factor alpha monoclonal antibody combinded with low-dose weekly methotrixate in rheumatoid arthritis. Arthritis Rheum 1998 September 41(9):1552-15563.
1. Dayer, J. M., et al., Production of collagenase and prostaglandins by isolated adherent rheumatoid synovial cells. Proc Natl Acad Sci USA, 1976. 73(3): p. 945-9.
2. Edwards, J. C., et al., The formation of human synovial joint cavities: a possible role for hyaluronan and CD44 in altered interzone cohesion. J Anat, 1994. 185(Pt 2): p. 355-67.
3. Burmester, G. R., et al., The tissue architecture of synovial membranes in inflammatory and non-inflammatory joint diseases. I. The localization of the major synovial cell populations as detected by monoclonal reagents directed towards Ia and monocyte-macrophage antigens. Rheumatol Int, 1983. 3(4): p. 173-81.
4. Hammerman, D., M. Stephens, and P. Barland, Comparative histology and metabolism of synovial tissue in normal arthritic joints, Inflammation and Disease of Connective Tissue., L. C. Mills and J. H. Moyer, Editors. 1961, W.B. Saunders: Philadelphia. p. 158-68.
5. Fox, R. I. and H. Kang, Structure and function of synoviocytes, in Arthritis and Allied Conditions, D. J. McCarthy and W. J. Koopman, Editors. 1993, Lea & Febiger: Malvern. p. 263-278.
6. Edwards, J. C., The nature and origins of synovium: experimental approaches to the study of synoviocyte differentiation. J Anat, 1994. 184(Pt 3): p. 493-501.
7. Cush, J. J. and P. E. Lipsky, Cellular basis for rheumatoid inflammation. Clin. Orthop., 1991. P 9-22: p. 1-22.
8. Seki, T., et al., Use of differential subtraction method to identify genes that characterize the phenotype of cultured rheumatoid arthritis synoviocytes. Arthritis Rheum, 1998. 41(8): p. 1356-64.
9. Okada, Y., et al., localization of type VI collagen in the lining cell layer of normal and rheumatoid synovium. Lab Invest, 1990. 63(5): p. 647-56.
10. Storm, E. E. and D. M. Kingsley, Joint patterning defects caused by single and double mutations in members of the bone morphogenetic protein (BMP) family. Development, 1996. 122(12): p. 3969-79.
11. Ritchlin, C. T. and R. J. Winchester, Potential mechanisms for coordinate gene activation in the rheumatoid synoviocyte: implications and hypotheses. Springer Semin Immunopathol, 1989. 11(3): p. 219-34.
12. Winchester, R., F. Su, and C. Ritchlin, Alteration of synoviocytes by inflammation—the source of a persistent non-immunologic drive in synovitis: analysis of levels of mRNA expression by a simple multi-gene assay. Clin Exp Rheumatol, 1993. 11 Suppl 8: p. S87-90.
13. Ritchlin, C., et al., Sustained and distinctive patterns of gene activation in synovial fibroblasts and whole synovial tissue obtained from inflammatory synovitis. Scand J Immunol, 1994. 40(3): p. 292-8.
14. Bucala, R., et al., Constitutive production of inflammatory and mitogenic cytokines by rheumatoid synovial fibroblasts. J Exp Med, 1991. 173(3): p. 569-74.
15. Winchester, R. J. and G. R. Burmester, Demonstration of Ia antigens on certain dendritic cells and on a novel elongate cell found in human synovial tissue. Scand J Immunol, 1981. 14(4): p. 439-44.
16. Klareskog, L., et al., Immune functions of human synovial cells. Phenotypic and T cell regulatory properties of macrophage-like cells that express HLA-DR. Arthritis Rheum, 1982. 25(5): p. 488-501.
17. Burmester, G. R., et al., Identification of three major synovial lining cell populations by monoclonal antibodies directed to Ia antigens and antigens associated with monocytes/macrophages and fibroblasts. Scand J Immunol, 1983. 17: p. 69-82.
18. Zvaifler, N. J., et al., Identification of immunostimulatory dendritic cells in the synovial effusions of patients with rheumatoid arthritis. J Clin Invest, 1985. 76(2): p. 789-800.
19. Burmester, G. R., et al., Ia+ T cells in synovial fluid and tissues of patients with rheumatoid arthritis. Arthritis Rheum, 1981. 24: p. 1370-1376.
20. Smith, C. A., Properties of synovial cells In culture. J Exp Med, 1971. 134: p. 306S.
21. Castor, C. W., Connective tissue activation. II. Abnormalities of cultured rheumatoid synovial cells. Arthritis Rheum, 1971. 14(1): p. 55-66.
22. Werb, Z., et al., Endogenous activiation of latent collagenase by rheumatoid synovial cells. Evidence for a role of plasminogen activator. N Engl J Med, 1977. 296(18): p. 1017-23.
23. Castor, C. W., et al., Connective tissue activation. XI. Stimulation of glycosaminoglycan and DNA formation by a platelet factor. Arthritis Rheum, 1977. 20(3): p. 859-68.
24. Imamura, F., et al., Monoclonal expansion of synoviocytes in rheumatoid arthritis [In Process Citation]. Arthritis Rheum, 1998. 41(11): p. 1979-86.
25. Kriegsmann, J., et al., Expression of vascular cell adhesion molecule-1 mRNA and protein in rheumatoid synovium demonstrated by in situ hybridization and immunohistochemistry. Lab Invest, 1995. 72(2): p. 209-14.
26. Ritchlin, C. and S. Haas-Smith, Collagenase and stromelysin expression in rheumatoid synovium and cartilage: comment on the article by Wolfe et al [letter; comment]. Arthritis Rheum, 1994. 37(12): p. 1831-3.
27. Winchester, R., Rheumatoid Arthritis, in Sampter's Immunological Diseases, M. M. Frank and K. F. Austen, Editors. 1995, Little, Brown and Company: Boston. p. 699-757.
28. Trabandt, A., et al., Expression of the collagenolytic and Ras-induced cysteine proteinase cathepsin L and proliferation-associated oncogenes in synovial cells of MRL/I mice and patients with rheumatoid arthritis. Matrix, 1990. 10(6): p. 349-61.
29. Hubank, M. and D. G. Schatz, Identifying differences in mRNA expression by representational difference analysis of cDNA. Nucleic Acids Res, 19.94. 22(25): p. 5640-8.
30. Lisitsyn, N. and M. Wigler, Cloning the differences between two complex genomes. Science, 1993. 259(5097). p. 946-51.
31. Hachicha, M., et al., Production of monocyte chemotactic protein-1 in human type B synoviocytes. Synergistic effect of tumor necrosis factor alpha and interferon-gamma. Arthritis Rheum, 1993. 36(1): p. 26-34.
32. Villiger, P. M., R. Terkeltaub, and M. Lotz, Production of monocyte chemoattractant protein-1 by inflamed synovial tissue and cultured synoviocytes. J Immunol, 1992. 149(2): p. 722-7.
33. Rathanaswami, P., et al., Expression of the cytokine RANTES in human rheumatoid synovial fibroblasts. Differential regulation of RANTES and interieukin-8 genes by inflammatory cytokines. J Biol Chem, 1993. 268(8): p. 5834-9.
34. Guerne, P. A., et al., Synovium as a source of interleukin 6 in vitro. Contribution to local and systemic manifestations of arthritis. J Clin Invest, 1989. 83(2): p. 585-92.
35. Firestein, G. S., et al., Quantitative analysis of cytokine gene expression in rheumatoid arthritis [published erratum appears in J Immunol 1990 Aug. 1; 145(3):1037]. J Immunol, 1990. 144(9): p. 3347-53.
36. Hermann, J. A., et al., Important immunoregulatory role of interleukin-11 in the inflammatory process in rheumatoid arthritis. Arthritis Rheum, 1998. 41(8): p. 1388-97.
37. Mino, T., et al.; Interleukin-1alpha and tumor necrosis factor alpha synergistically stimulate prostaglandin E2-dependent production of interleukin-11 in rheumatoid synovial fibroblasts [In Process Citation]. Arthritis Rheum, 1998. 41(11): p. 2004-13.
38. Firestein, G. S. and M. M. Paine, Stromelysin and tissue inhibitor of metalloproteinases gene expression in rheumatoid arthritis synovium. Am J Pathol, 1992. 140(6): p. 1309-14.
39. McCachren, S. S., Expression of metalloproteinases and metalloproteinase inhibitor in human arthritic synovium. Arthritis Rheum, 1991. 34(9): p. 1085-93.
40. Wicks, I. P., et al., The effect of cytokines on the expression of MHC antigens and ICAM-1 by normal and transformed synoviocytes. Autoimmunity, 1992. 12(1): p. 13-9.
41. Bombara, M. P., et al., Cell contact between T cells and synovial fibroblasts causes induction of adhesion molecules and cytokines. J Leukoc Biol, 1993. 54(5): p. 399-406.
42. Chin, J. E., et al., Role of cytokines in inflammatory synovitis. The coordinate regulation of intercellular adhesion molecule 1 and HLA class I and class II antigens in rheumatoid synovial fibroblasts. Arthritis Rheum, 1990. 33(12): p. 1776-86.
43. Demaziere, A. and N. A. Athanasou, Adhesion receptors of intimal and subintimal cells of the normal synovial membrane. J Pathol, 1992. 168(2): p. 20.9-15.
44. Lindsley, H. B., et al., Regulation of the expression of adhesion molecules by human synoviocytes. Semin Arthritis Rheum, 1992. 21(5): p. 330-4.
45. Rinaldi, N., et al., Increased expression of integrins on fibroblast-like synoviocytes from rheumatoid arthritis in vitro correlates with enhanced binding to extracellular matrix proteins. Ann Rheum Dis, 1997. 56(1): p. 45-51.
46. Croft, D. R., et al., Complex CD44 splicing combinations in synovial fibroblasts from arthritic joints. Eur J Immunol, 1997. 27(7): p. 1680-4.
47. Henderson, K. J., J. C. Edwards, and J. G. Worrall, Expression of CD44 in normal and rheumatoid synovium and cultured synovial fibroblasts. Ann Rheum Dis, 1994. 53(11): p. 729-34.
48. Yellin, M. J., et al., Ligation of CD40 on fibroblasts induces CD54 (ICAM-1) and CD106 (VCAM-1) up-regulation and IL-6 production and proliferation. J Leukoc Biol, 1995. 58(2): p. 209-16.
49. Seitz, M., et al., Production of interleukin-1 receptor antagonist, inflammatory chemotactic proteins, and prostaglandin E by rheumatoid and osteoarthritic synoviocytes—regulation by IFN-gamma and IL-4. J Immunol, 1994. 152(4): p. 2060-5.
50. Hogan, M., et al., Differential expression of the small inducible cytokines GRO alpha and GRO beta by synovial fibroblasts in chronic arthritis: possible role in growth regulation. Cytokine, 1994. 6(1): p. 61-9.
51. Koch, A. E., et al., Macrophage inflammatory protein-1 beta: a C-C chemokine in osteoarthritis. Clin Immunol Immunopathol, 1995. 77(3): p. 307-14.
52. Campbell, J. J., et al., Chemokines and the arrest of lymphocytes rolling under flow conditions. Science, 1998. 279(5349): p. 381-4.
53. Moser, B., et al., Lymphocyte responses to chemokines. Int Rev Immunol, 1998. 16(3-4): p. 323-44.
54. Dunon, D., L. Piali, and B. A. Imhof, To stick or not to stick: the new leukocyte homing paradigm. Curr Opin Biol, 1996. 8: p. 714-723.
55. Kim, C. H., et al., CK beta-11/macrophage inflammatory protein-3 beta/EBI1-ligand chemokine is an efficacious chemoattractant for T and B cells. J Immunol, 1998. 160(5): p. 2418-24.
56. Delgado, E., et al., Mature dendritic cells respond to SDF-1, but not to several beta-chemokines. Immunobiology, 1998. 198(5): p. 490-500.
57. Nagasawa, T., et al., Defects of B-cell lymphopoiesis and bone-marrow myelopoiesis in mice lacking the CXC chemokine PBSF/SDF-1. Nature, 1996. 382(6592): p. 635-8.
58. Zou, Y. R., et al., Function of the chemokine receptor CXCR4 in haematopoiesis and in cerebellar development [see comments]. Nature, 1998. 393(6685): p. 595-9.
59. Bonecchi, R., et al., Differential expression of chemokine receptors and chemotactic responsiveness of type 1 T helper cells (Thls) and Th2s. J Exp Med, 1998. 187(1): p. 129-34.
60. Qin, S., et al., The chemokine receptors CXCR3 and CCR5 mark subsets of T cells associated with certain inflammatory reactions. J Clin Invest, 1998. 101(4): p. 746-54.
61. Bedard, P. A. and E. E. Golds, Cytokine-induced expression of mRNAs for chemotactic factors in human synovial cells and fibroblasts. J Cell Physiol, 1993. 154(2): p. 433-41.
62. Hosaka, S., et al., Expression of the chemokine superfamily in rheumatoid arthritis. Clin. Exp Immunol, 1994. 97(3): p. 451-7.
63. Barnes, D. A., et al., Polyclonal antibody directed against human RANTES ameliorates disease in the Lewis rat adjuvant-induced arthritis model. J Clin Invest, 1998. 101(12): p. 2910-9.
64. Brennan, F. M., et al., Cytokine production in culture by cells isolated from the synovial membrane. J Autoimmun 1989. 2 Suppl: p. 177-86.
65. Buchan, G., et al., Interleukin-1 and tumour necrosis factor mRNA expression in rheumatoid arthritis prolonged production of IL-1 alpha. Clin Exp Immunol, 1988. 73(3): p. 449-55.
66. Alvaro-Gracia, J. M., et al., Cytokines in chronic inflammatory arthritis. VI. Analysis of the synovial cells involved in granulocyte-macrophage colony-stimulating factor production and gene expression in rheumatoid arthritis and its regulation by IL-1 and tumor necrosis factor-alpha. J Immunol, 1991. 146(10): p. 3365-71.
67. Okamoto, H., et al., The synovial expression and serum levels of interleukin-6, interleukin-11, leukemia inhibitory factor, and oncostatin M in rheumatoid arthritis. Arthritis Rheum, 1997. 40(6): p. 1096-105.
68. Tan, P. L., et al., Expression of the interleukin 6 gene in rheumatoid synovial fibroblasts. J Rheumatol, 1990. 17(12): p. 1608-12.
69. McInnes, I. B., et al., The role of interleukin-15 in T-cell migration and activation in rheumatoid arthritis. Nat Med, 1996. 2(2): p. 175-82.
70. Temime, N., et al., Autocrine stimulation of interleukin 1 in human adherent synovial lining cells: down regulation by interferon gamma. Hum Immunol, 1991. 31(4): p. 261-70.
71. Case, J. P., et al., IL-1 regulation of transin/stromelysin transcription in rheumatoid synovial fibroblasts appears to involve two antagonistic transduction pathways, an inhibitory, prostaglandin dependent pathway mediated by cAMP, and a stimulatory, protein kinase C-dependent pathway. J Immunol, 1990. 145(11): p. 3755-61.
72. Goddard, D. H., S. L. Grossman, and M. E. Moore, Autocrine regulation of rheumatoid arthritis synovial cell growth in vitro. Cytokine, 1990. 2(2): p. 149-55.
73. Koch, A. E., et al., Synovial tissue macrophage as a source of the chemotactic cytokine IL-8. J Immunol, 1991. 147(7): p. 2187-95.
74. Krane, S. M., et al., Mononuclear cell-conditioned medium containing mononuclear cell factor (MCF), homologous with interleukin 1, stimulates collagen and fibronectin synthesis by adherent rheumatoid synovial cells: effects of prostaglandin E2 and indomethacin. Coll Relat Res, 1985. 5(2): p. 99-117.
75. Alvaro-Gracia, J. M., N. J. Zvaifler, and G. S. Firestein, Cytokines in chronic inflammatory arthritis. V. Mutual antagonism between interferon-gamma and tumor necrosis factor-alpha on HLA-DR expression, proliferation, collagenase production, and granulocyte macrophage colony-stimulating factor production by rheumatoid arthritis synoviocytes. J Clin Invest, 1990. 86(6): p. 1790-8.
76. Lemaire, R., et al., Selective induction of the secretion of cathepsins B and L by cytokines in synoval fibroblast-like cells. Br J Rheumatol, 1997. 36(7): p. 735-43.
77. Keffer, J., et al., Transgenic mice expressing human tumour necrosis factor: a predictive genetic model of arthritis. Embo J, 1991. 10(13): p. 4025-31.
78. Moreland, L. W., et al., Treatment of rheumatoid arthritis with a recombinant human tumor necrosis factor receptor (p75)-Fc fusion protein [see comments]. N Engl J Med, 1997. 337(3): p. 141-7.
79. Mori, L., et al., Attenuation of collagen-induced arthritis in 55-kDa TNF receptor type 1 (TNFR1)-IgG1-treated and TNFR1-deficient mice. J Immunol, 1996. 157(7): p. 3178-82.
80. Elliott, M. J., et al., Randomised double-blind comparison of chimeric monoclonal antibody to tumour necrosis factor alpha (cA2) versus placebo in rheumatoid arthritis. Lancet, 1994. 344(8930): p. 1105-10.
81. Alonzi, T., et al., Interleukin 6 is required for the development of collagen-induced arthritis. J Exp Med, 1998. 187(4): p. 461-8.
82. Mihara, M., et al., Interleukin-6 (IL-6) induces the proliferation of synovial fibroblastic cells in the presence of soluble IL-6 receptor. Br J Rheumatol, 1995. 34(4): p. 321-5.
83. Ito, A., et al., Effects of interleukin-6 on the metabolism of connective tissue components in rheumatoid synovial fibroblasts. Arthritis Rheum, 1992. 35(10): p. 1197-201.
84. Lotz, M. and P. A. Guerne, Interleukin-6 induces the synthesis of tissue inhibitor of metalloproteinases-1/erythroid potentiating activity (TIMP-1/EPA). J Biol Chem, 1991. 266(4): p. 2017-20.
85. Richards, C. D., et al., Selective regulation of metalloproteinase inhibitor (TIMP-1) by oncostatin M in fibroblasts in culture. J Immunol, 1993. 150(12): p. 5596-603.
86. Lotz, M., T. Moats, and P. M. Villiger, Leukemia inhibitory factor is expressed in cartilage and synovium and can contribute to the pathogenesis of arthritis. J Clin Invest, 1992. 90(3): p. 888-96.
87. Langdon, C., et al., Oncostatin M stimulates monocyte chemoattractant protein-1- and interleukin-1-induced matrix metalloproteinase-1 production by human synovial fibroblasts in vitro. Arthritis Rheum, 1997. 40(12): p. 2139-46.
88. Cawston, T. E., et al., The role of oncostatin M in animal and human connective tissue collagen turnover and its localization within the rheumatoid joint [In Process Citation]. Arthritis Rheum, 1998. 41(10): p. 1760-71.
89. Gulko, P., et al., Identification of a new non-MHC genetic locus on chromosome 2 controlling disease severity in collagen-induced arthritis in rats. Arthrititis Rheum, 1998. In press.
90. McInnes, I. B., et al., Interleukin-15 mediates T cell-dependent regulation of tumor necrosis factor-alpha production in rheumatoid arthritis [see comments]. Nat Med, 1997. 3(2): p. 189-95.
91. Hamilton, J. A., et al., Cytokine regulation of colony-stimulating factor (CSF) production in cultured human synovial fibroblasts. II. Similarities and differences in the control of interleukin-1 induction of granulocyte-macrophage CSF and granuiocyte-CSF production. Blood, 1992. 79(6): p. 1413-9.
92. Campbell, I. K., et al., Protection from collagen-induced arthritis in granulocyte-macrophage colony-stimulating factor-deficient mice. J Immunol, 1998. 161(7): p. 3639-44.
93. Chu, C. Q., et al., Transforming growth factor-beta 1 in rheumatoid synovial membrane and cartilage/pannus junction. Clin Exp Immunol, 1991. 86(3): p. 380-6.
94. Mangasser-Stephan, K., et al., Identification of human semaphorin E gene expression in rheumatoid synovial cells by mRNA differential display. Biochem Biophys Res Commun, 1997. 234(1): p. 153-6.
95. Luo, Y., et al., A family of molecules related to collapsin in the embryonic chick nervous system [published erratum appears in Neuron 1995 November; 15(5):following 1218]. Neuron, 1995. 14(6): p. 1134-40.
96. Kidd, B. L., et al., A neurogenic mechanism for symmetrical arthritis [see comments]. Lancet, 1989. 2(8672): p. 1128-30.
97. Sekido, Y., et al., Human semaphorins A(V) and IV reside in the 3p21.3 small cell lung cancer deletion region and demonstrate distinct expression patterns. Proceedings of the National Academy of Sciences of the United States of America, 1996. 93: p. 4120-5.
98. Yamada, T., et al., Identification of semaphorin E as a non-MDR drug resistance gene of human cancers. Proc Natl Acad Sci USA, 1997. 94(26): p. 14713-8.
99. Koths, K., et al., Cloning and characterization of a human Mac-2-binding protein, a new member of the superfamily defined by the macrophage scavenger receptor cysteine-rich domain. J Biol Chem, 1993. 268(19): p. 14245-9.
100. Inohara, H., et al., Interactions between galectin-3 and Mac-2-binding protein mediate cell-cell adhesion. Cancer Res, 1996. 56(19): p. 4530-4.
101. Yu, B. and S. D. Wright, LPS-dependent interaction of Mac-2-binding protein with immobilized CD14. J Inflamm, 1995. 45(2): p. 115-25.
102. Jeng, K. C., L. G. Frigeri, and F. T. Liu, An endogenous lectin, galectin-3 (epsilon BP/Mac-2), potentiates IL-1 production by human monocytes. Immunol Lett, 1994% 42(3): p. 113-6.
103. Merrill, J., et al., Synovial fibroblast supernatants induce monocytes to form giant cells via CD18 integrins. Arthritis & Rheumatism, 1992. 35: p. S97.
104. Valencia, X., et al., Identification of cadherin-11 in type B synoviocytes derived from rheumatoid arthritis patients. Arthritis Rheum, 1998. 41 (supplement): p. S190.
105. Rada, J. A., P. K. Cornuet, and J. R. Hassell, Regulation of corneal collagen fibrillogenesis in vitro by corneal proteoglycan (lumican and decorin) core proteins. Exp Eye Res, 1993. 56(6): p. 635-48.
106. Funderburgh, J. L., et al., Macrophage receptors for lumican. A corneal keratan sulfate proteoglycan. Invest Ophthalmol Vis Sci, 1997. 38(6): p. 1159-67.
107. Hildebrand, A., et al., Interaction of the small interstitial proteoglycans biglycan, decorin and fibromodulin with transforming growth factor beta. Biochem J, 1994. 302(Pt 2): p. 527-34.
108. Ungefroren, H. and N. B. Krull, Transcriptional regulation of the human biglycan gene. J Biol Chem, 1996. 271(26): p. 15787-95.
109. McKee, C. M., et al., Hyaluronan (HA) fragments induce chemokine gene expression in alveolar macrophages. The role of HA size and CD44. J Clin Invest, 1996. 98 (10) p 0.2403-13.
110. Jones, J. I., et al., Extracellular matrix contains insulin-like growth factor binding protein-5: potentiation of the effects of IGF-I. J Cell Biol, 1993. 121(3): p. 679-87.
111. Tyler, J. A., Insulin-like growth factor 1 can decrease degradation and promote synthesis of proteoglycan in cartilage exposed to cytokines. Biochem J, 1989. 260(2): p. 543-8.
112. Pash, J. M. and E. Canalis, Transcriptional regulation of insulin-like growth factor-binding protein-5 by prostaglandin E2 in osteoblast cells. Endocrinology, 1996. 137(6): p. 2375-82.
113. Remmers, E. F., H. Sano, and R. L. Wilder, Platelet-derived growth factors and heparin-binding (fibroblast) growth factors in the synovial tissue pathology of rheumatoid arthritis. Semin Arthritis Rheum, 1991. 21(3): p. 191-9.
114. Marie, I. and A. G. Hovanessian, The 69-kDa 2-5A synthetase is composed of two homologous and adjacent functional domains. J Biol Chem, 1992. 267(14): p. 9933-9.
115. Wathelet, M., et al., Molecular cloning, full-length sequence and preliminary characterization of a 56-kDa protein induced by human interferons. Eur J Biochem, 1986. 155(1): p. 11-7.
116. Burmester, G. R., et al., Differential expression of Ia antigens by rheumatoid synovial lining cells. J Clin Invest, 1987. 80(3): p. 595-604.
117. Edwards, J. C., et al., Matrix metalloproteinases in the formation of human synovial joint cavities. J Anat, 1996. 188(Pt 2): p. 355-60.
118. Gravallese, E. M., et al., In situ hybridization studies of stromelysin and collagenase messenger RNA expression in rheumatoid synovium. Arthritis Rheum, 1991. 34(9): p. 1076-84.
119. Woolley, D. E., M. J. Crossley, and J. M. Evanson, Collagenase at sites of cartilage erosion in the rheumatoid joint. Arthritis Rheum, 1977. 20(6): p. 1231-9.
120. Goldberg, G. I., et al., Human fibroblast collagenase. Complete primary structure and homology to an oncogene transformation-induced rat protein. Journal of Biological Chemistry, 1986. 261: p. 6600-6605.
121. Suzuki, K., et al., Mechanisms of activation of tissue procollagenase by matrix metalloproteinase 3 (stromelysin). Biochemistry, 1990. 29(44): p. 10261-70.
122. Cawston, T. E., et al., The interaction of purified rabbit bone collagenase with purified rabbit bone metalloproteinase inhibitor. Biochemical Journal, 1983. 211: p. 313-318.
123. Stetler Stevenson, W. G., et al., Tissue inhibitor of metalloproteinases-2 (TIMP-2) mRNA expression in tumor cell lines and human tumor tissues. J Biol Chem, 1990. 265: p. 13933-13938.
124. Okada, Y., et al., Matrix metalloproteinase 2 from human rheumatoid synovial fibroblasts. Purification and activation of the precursor and enzymic properties. Eur J Biochem, 1990. 194(3): p. 721-30.
125. Ritchlin, C., et al., Patterns of gene activation in synovial fibroblasts and whole synovial tissue obtained from patients with inflammatory and non-inflammatory synovitis. Clinical Research, 1991. 39: p. 341.
126. Mellors, R. C. and et. al., Rheumatoid factor and the pathogenesis of rheumatoid arthritis. Journal of Experimental Medicine, 1961. 113: p. 475.
127. Oritani, K. and P. W. Kincade, Identification of stromal cell products that interact with pre-B cells. J Cell Biol, 1996. 134(3): p. 771-82.
128. Fujisawa, K., et al., Activation of transcription factor NF-kappa B in human synovial cells in response to tumor necrosis factor alpha. Arthritis Rheum, 1996. 39(2): p. 197-203.
129. Miagkov, A. V., et al., NF-kappaB activation provides the potential link between inflammation and hyperplasia in the arthritic joint [In Process Citation]. Proc Natl Acad Sci USA, 1998. 95(23): p. 13859-64.
130. Muller-Ladner, U., et al., Oncogenes in rheumatoid arthritis. Rheum Dis Clin North Am, 1995. 21(3): p. 675-90.
131. Trabandt, A., et al., Spontaneous expression of immediately-early response genes c-fos and egr-1 in collagenase-producing rheumatoid synovial fibroblasts. Rheumatol Int, 1992. 12(2): p. 53-9.
132. Aicher, W. K., et al., Overexpression of zinc-finger transcription factor Z-225/Egr-1 in synoviocytes from rheumatoid arthritis patients [published erratum appears in J Immunol 1994 Dec. 1; 153(11):5347]. J Immunol, 1994. 152(12): p. 5940-8.
133. Dooley, S., et al., Constitutive expression of c-fos and c-jun, overexpression of ets-2, and reduced expression of metastasis suppressor gene nm23-H1 in rheumatoid arthritis. Ann Rheum Dis, 1996. 55(5): p. 298-304.
134. Morita, Y., et al., Antisense oligonucleotides targeting c-fos mRNA inhibit rheumatoid synovial fibroblast proliferation. Ann Rheum Dis, 1998. 57(2): p. 122-4.
135. Shiozawa, S., et al., Studies on the contribution of c-fos/AP-1 to arthritic joint destruction. J Clin Invest, 1997. 99(6): p. 1210-6.
136. Roivainen, A., et al., H-ras oncogene point mutations in arthritic synovium. Arthritis Rheum, 1997. 40(9): p. 1636-43.
137. Qu, Z., et al., Local proliferation of fibroblast-like synoviocytes contributes to synovial hyperplasia. Results of proliferating cell nuclear antigen/cyclin, c-myc, and nucleolar organizer region staining [see comments]. Arthritis Rheum, 1994. 37(2): p. 212-20.
138. Remmers, E. F., et al., Production of platelet derived growth factor B chain (PDGF-B/c-sis) mRNA and immunoreactive PDGF B-like polypeptide by rheumatoid synovium: coexpression with heparin binding acidic fibroblast growth factor-1. J Rheumatol, 1991. 18(1): p. 7-13.
139. Nakajima, T., et. al., Apoptosis and functional Fas antigen in rheumatoid arthritis synoviocytes. Arthritis Rheum, 1995. 38(4): p. 485-91.
140. Firestein, G. S., M. Yeo, and N. J. Zvaifler, Apoptosis in rheumatoid arthritis synovium. J Clin Invest, 1995. 96(3): p. 1631-8.
141. Hashimoto, H., et al., Soluble Fas ligand in the joints of patients with rheumatoid arthritis and osteoarthritis. Arthritis Rheum, 1998. 41(4): p. 657-62.
142. Fujisawa, K., et al., Therapeutic effect of the anti-Fas antibody on arthritis in HTLV-1 tax transgenic mice. J. Clin. Invest, 1996. 98(2): p. 271-8.
143. Zhang, H., et al., Amelioration of collagen-induced arthritis by CD95 (Apo-1/Fas)-ligand gene transfer. J Clin Invest, 1997. 100(8): p. 1951-7.
144. Wakisaka, S., et al., Modulation by proinflammatory cytokines of Fas/Fas ligand-mediated apoptotic cell death of synovial cells in patients with rheumatoid arthritis (RA) [In Process Citation]. Clin Exp Immunol, 1998. 114(1): p. 0.119-28.
145. Kawakami, A., et al., Inhibition of Fas antigen-mediated apoptosis of rheumatoid synovial cells in vitro by transforming growth factor beta 1. Arthritis Rheum, 1996. 39(8): p. 1267-76.
146. Firestein, G. S., et al., Apoptosis in rheumatoid arthritis: p53 overexpression in rheumatoid arthritis synovium. Am J Pathol, 1996. 149(6): p. 2143-51.
147. Firestein, G. S., et al., Somatic mutations in the p53 tumor suppressor gene in rheumatoid arthritis synovium. Proc Natl Acad Sci USA, 1997. 94(20): p. 10895-900.
148. Aupperle, K. R., et al., Regulation of synoviocyte proliferation, apoptosis, and invasion by the p53 tumor suppressor gene. Am J Pathol, 1998. 152(4): p. 1091-8.
149. Van Antwerp, D. J.; et al., Suppression of TNF-alpha-induced apoptosis by NF-kappaB [see comments]. Science, 1996. 274(5288): p. 787-9.
150. Scheinman, R. I., et al., Role of transcriptional activation of I kappa B alpha in mediation of immunosuppression by glucocorticoids [see comments]. Science, 1995. 270(5234): p. 283-6.
151. Auphan, N., et al., Immunosuppression by glucocorticoids: inhibition of NF-kappa B activity through induction of kappa B synthesis [see comments]. Science, 1995. 270(5234): p. 286-90.
152. Yang, J. P., et al., Inhibition of the DNA-binding activity of NF-kappa B by gold compounds in vitro. FEBS Lett, 1995. 361(1): p. 89-96.
153. Isomaki, P., et al., Expression of bcl-2 in rheumatoid arthritis. Br J Rheumatol, 1996. 35(7): p. 611-9.
154. Matsumoto, S., et al., Ultrastructural demonstration of apoptosis, Fas and Bcl-2 expression of rheumatoid synovial fibroblasts. J Rheumatol, 1996. 23(8): p. 1345-52.
155. Dingle, J. T., Heberden oration 1978. Recent studies on the control of joint damage: the contribution of the Strangeways Research Laboratory. Ann Rheum Dis, 1979. 38(3): p. 201-14.
156. Steinberg, J., et al., A tissue-culture model of cartilage breakdown in rheumatoid arthritis. Quantitative aspects of proteoglycan release. Biochem J, 1979. 180(2): p. 403-12.
157. Harris, E. D., Jr., D. R. DiBona, and S. M. Krane, Amechanism for cartilage destruction in rheumatoid arthritis. Trans. Assoc. Am. Physicians, 1970. 83: p. 267.
158. Kingsley-Mills, W. M., Pathology of the knee joint in rheumatoid arthritis. Journal of Bone & Joint Surgery 1970. 52: p. 746.
159. Saxne, T. and D. Heinegard, Synovial fluid analysis of two groups of proteoglycan epitopes distinguishes early and late cartilage lesions. Arthritis Rheum, 1992. 35(4): p. 385-90.
160. Mudgett, J. S., et al., Susceptibility of stromelysin 1-deficient mice to collagen-induced arthritis and cartilage destruction. Arthritis Rheum, 1998. 41(1): p. 110-21.
161. Muller-Ladner, U., et al., Synovial fibroblasts of patients with rheumatoid arthritis attach to and invade normal human cartilage when engrafted into SCID mice. Am J Pathol, 1996. 149(5): p. 1607-15.
162. Geiler, T., et al., A new model for rheumatoid arthritis generated by engraftment of rheumatoid synovial tissue and normal human cartilage into SCID mice. Arthritis Rheum, 1994. 37(11): p. 1664-71.
163. Shiozawa, S., et al., Destructive arthritis without lymphocyte infiltration in H2-c-fos transgenic mice. J Immunol, 1992. 148(10): p. 3100-4.
164. Scott, B. B., et al., Rheumatoid arthritis synovial fibroblast and U937 macrophage/monocyte cell line interaction in cartilage degradation. Arthritis Rheum, 1997. 40(3): p. 490-8.
165. Velculescu, V. E., et al., Serial analysis of gene expression [see comments]. Science, 1995. 270(5235): p. 484-7.
166. Heller, R. A., et al., Discovery and analysis of inflammatory disease-related genes using cDNA microarrays. Proc Natl Acad Sci USA, 1997. 94(6): p. 2150-5.
167. Dayer, J. M., et al., Participation of monocyte-macrophages and lymphocytes in the production of a factor that stimulates collagenase and prostaglandin release by rheumatoid synovial cells. Journal of Clinical Investigation, 1979. 64: p. 1386-1392.
168. Koch, A. E., P. J. Polyerini, and S. J. Leibovich, Stimulation of neovascularization by human rheumatoid synovial tissue macrophages. Arthritis Rheum, 1986. 29(4): p. 471-9.
169. Lorentzen, J. C., et al., Identification of rat susceptibility loci for adjuvant oil-induced arthritis. Proc Natl Acad Sci USA, 1998. 95(11): p. 6383-7.
170. Kawahito, Y., et al., Localization of quantitative trait loci regulating adjuvant induced arthritis in rats: evidence for genetic factors common to multiple autoimmune diseases. J Immunol, 1998. 161(8): p. 4411-9.
171. Remmers, E. F., et al., A genome scan localizes five non-MHC loci controlling collagen-induced arthritis in rats. Nat Genet, 1996. 14(1): p. 82-5.
172. Cornelis, F., et al., New susceptibility locus for rheumatoid arthritis suggested by a genome-wide linkage study [In Process Citation]. Proc Natl Acad. Sci USA, 1998. 95(18): p. 10746-50.

1. A method for treating rheumatoid arthritis or other form of inflammatory arthritis which comprises administering to a subject an amount of an agent effective to inhibit the activation of the CXCR4 receptor by SDF-1.

2. The method of claim 1, wherein the agent is an oligopeptide or a polypeptide.

3. The method of claim 1, wherein the agent is an antibody or a portion of an antibody.

4. The method of claim 3, wherein the antibody is a human antibody, a chimeric antibody or a humanized antibody.

5. The method of claim 1, wherein the agent is a nonpeptidyl agent.

6. The method of claim 5, wherein the nonpeptidyl agent is a bicyclam.

7-17. (canceled)


SEQUENCE LISTING (23 sequences)

SEQ ID NO: 1 - DNA, 13 bp, Artificial Sequence
  Primer
gatccgcggccgc

SEQ ID NO: 2 - DNA, 10 bp, Artificial Sequence
  Primer
gcggccgcgt

SEQ ID NO: 3 - DNA, 24 bp, Artificial Sequence
  Primer
accgacgtcgactatccatgaacg

SEQ ID NO: 4 - DNA, 12 bp, Artificial Sequence
  Primer
gatccgttcatg

SEQ ID NO: 5 - DNA, 24 bp, Artificial Sequence
  Primer
aggcaactgtgctatccgagggag

SEQ ID NO: 6 - DNA, 12 bp, Artificial Sequence
  Primer
gatcctccctcg

SEQ ID NO: 7 - DNA, 24 bp, Artificial Sequence
  Primer
agcactctccagcctctcaccgag

SEQ ID NO: 8 - DNA, 12 bp, Artificial Sequence
  Primer
gatcctcggtga

SEQ ID NO: 9 - protein, 507 aa, mouse
  MISC_FEATURE (337)..(337): x= to any amino acid
SAVCVYHLSDIQTVFNGPFAHKEGPNHQLISYQGRIPYPRSAVCVYHLSDIQTVFNGPFAHKEGPNHQLISYQGRIPYPRSAVCVYSMADIRMVFNGPFAHKEGPNYQWMPFSGKMPYPRSAVCVYSMNDVRRAFLGPFAHKEGPMHQWVSYQGRVPYPRSAVCMYSMSDVRRVRRVFLGPYAHRDGPNYQWVPYQGRVPYPRPGTCPGGAFTPNMRTTKDFPDDVVTFIRNHPLMYNSISPIPGTCPGGALTPNMRTTKEFPDDVVTFIRNHPLMYNSIYPIPGTCPGGTFTPSMKSTKDYPDEVINFMRSHPLMYQAVYPLPGMCPSKTFGTFSXSTKDFPDDVIFARNHPLMYNSVLPTPGTCPSKTFGGFDXSTKDLPDDVITFARSHPAMYNPVFPMHRRPLIVRIGTDYKYTKIAVDHKRPLIVRIGTDYKYTKIAVDQRRPLVVRTGAPYRLTTIAVDGGRPLFLQVGANYTFTQIAADNNRPIVIKTDVNYQFTQIVVD

SEQ ID NO: 10 - protein, 396 aa, Human
SYPAPHGPEDPAPQFAHMFENEISHRTGSWNFAPNPDKQWLLQRTSHAAPHGPEDSAPQFSELYPNASQHITPSYNYAPNMDKHWIMQYTATPAPHSPWTAAPQYQKAFQNVFAPRNKNFNIHGTNKHWLIRQAKGKMNDVHISFTDLLHRRRLQTLQSVDEGIERLFNLLRELNQLWNTGPMLPIHMEFTNILQRKRLQTLMSVDDSVERLYNMLVETGELENTTPMTNSSIQFLDNAFRKRWQTLLSVDDLVEKLVKRLEFTGELNNTYAIYTSDHGYHLGQFGLLKGKNMPYEFDIRVPFFMRGPGIPRYIIYTADHGYHIGQFGLVKGKSMPYDFDIRVPFFIRGPSVEPYIFYTSDNGYHTGQFSLPIDKRQLYEFDIKVPLLVRGPGIKP

SEQ ID NO: 11 - protein, 102 aa, Human
SAVCVYYSMADIRMVFNGPFAHKEGPNYQWMPFSGKMPYPRPGTCPGGTFTPSMKSTKBYPDEVINFMRSHPLMYQAVYPLQRRPLVVRTGAPYRLTTIAVD

SEQ ID NO: 12 - protein, 101 aa, Human
  MISC_FEATURE (54)..(54): X= to any amino acid
SAVCVYSMNDVRRAFLGPFAHKEGPMHQWVSYQGRVPYPRPGMCPSKTFGTFSXSTKDFPDDVIQFARNHPKMYNSVLPTGGRPLFLQVGANYTFTQIAAD

SEQ ID NO: 13 - protein, 101 aa, Human
  MISC_FEATURE (54)..(54): X=to any amino acid
SAVCMYSMSDVRRVFLGPYAHRDGPNYQWVPYQGRVPYPRPGTCPSKTFGGFDXSTKDLPDDVITFARSHPAMYNPVFPMNNRPIVIKTDVNYQFTQIVVD

SEQ ID NO: 14 - protein, 90 aa, Worm
SYPAPHGPEDPAPQFAHMFENEISHRTGSWNFAPNPDKQWLLQRTGKMNDVHISFTDLLHRRRLQTLQSVDEGIERLFNLLRELNQLWNT

SEQ ID NO: 15 - protein, 132 aa, Worm
SHAAPHGPEDSAPQFSELYPNASQHITPSYNYAPNMDKHWIMQYTGPMLPIHMEFTNILQRKRLQTLMSVDDSVERLYNMLVETGELENTYIIYTADHGYHIGQFGLVKGKSMPYDFDIRVPFFIRGPSVEP

SEQ ID NO: 16 - protein, 130 aa, Human
ATPAPHSPWTAAPQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKP

SEQ ID NO: 17 - protein, 410 aa, Human
GNNGAGTGTGGGACGGGGGGNGNAGNAATTAAGGTAGNGATGGAGNANGGGGTGCNTNGGNNNAGANANTGNNTGGAGAANGACAANGGGGGNGTCGNNGGAGCNGNTGTGAGTGGGAAGAAGGCNACGTCAANAAGGACGAATATTTGCAANGNNGNNCAGGGCTGTNCNCGGGCAGTTTGTAAAAAAAAAAANAAGAACNGCGACAGACAAGTGTNNGTTGACCCGAAGCNANAGTGGATNCAGGAGTACCTGGAGNNAACTATGAACAANTAAGCGCAACAGCCAAAGAGGACTTNCCGCTAGACCCACTCGAGGAAAACTAAAACCTTGTGAGAGATGAAAGGNCAAAGACGTGGGGGAGGGGGCCNTAACCATGAGGACCAGGTGTGTGTGTGTGTGGGGTGGGC

SEQ ID NO: 18 - protein, 425 aa, Human
CCCGGGTACCGAGCTCGAATTCCGTTGNTGTCGCCGTTGNTGTCGCAGATGCCCATGCCCATGCCGATTCTTCGAAAGCCATGTTGCCAGAGCCAACGTCAAGCATCTCAAAATTCTCAACACTCCAAACTGTGCCCTTCAGATTGTAGCCCGGCTGAAGAACAACAACAGACAAGTGTGCATTGACCCGAAGCTAAAGTGGATTCAGGAGTACCTGGAGAAAGCTTTAAACAAGTAAGCACAACAGCCAAAAAGGACTTTCCGCTAGACCCANTCGAGAAAACTAAAACCTTGTGAGAGATGAAAGGGCAAAGACGTGGGGGGAGGGGGGCTTAACCATGAGGACCAGGTGTGTGTGTNGGGTGGGGCACATTGGATCTTNGATCGGGCCTGAGGTTTGGCAGCATTTAGACCCTGGATTATGN

SEQ ID NO: 19 - protein, 376 aa, Human
CAGATGCCCATGCCGATTCTTCGAAAGCCATGTTGCCAGAGCCAACGTCAAGCATCTCAAAATTCTCAACACTCCAAACTGTGCCCTTCAGATTGTAGCCCGGCTGAAGAACAACAACAGACAAGTGTGCATTGACCCGAAGCTAAAGTGGATTCAGGAGTACCTGGAGGAAAGCTTTAAACAAGTAAGCACAACAGCCAAAAAGGACTTTCCGCTAGACCCACTCGAGGAAAACTAAAACCTTGTGAGAGATGAAAGGGCAANGACGTNGNGGAGGGGGGCTTAACCATGAGGACCAGGTGTGTNTGGGGGTGGGTACATTGNATCTTGGGATCGGGCCTGAGGTTNGGCAGAATTTNGNCCCTGNATTTNTGGN

SEQ ID NO: 20 - protein, 377 aa, Human
CAGATGNCCATGCCGATTCTTCGAAAGCCATGTTGCCAGAGCCAACGTCAAGCATCTCAAAATTCTCAACACTCCAAACTGTGCCCTTCAGATTGTAGCCCGGCTGAAGAACAACAACAGACAAGTGTGCATTGACCCGAAGCTAAAGTGGATTCAGGAGTACCTGGAGTAAAGCTTTAAACAAGTAAGCACAACAGNCAAAAAGGACTTTCCGCTAGACCCACTCGAGGAAAACTAAAACCTTGTGAGAGATGAAAGGGCANTGTTNTTGTGGAGGGGGCCTTAACCATGAGGACCAGGTGTGTGTGTGGGGTGGGCACATNGNATCTGGGTATCGGGCCTGAGGTTTGNCAGCATTTAGNCCCTGNATTTATNGC

SEQ ID NO: 21 - protein, 292 aa, Human
CCATGTTCCAAGANCCACGTCAACATCCCAAAATCTCAACACNCCCAACTNTTCCCTTCAGATTGTAGCCCGGCTGAAGAACAACAACAAGACAAGTGTGCATTTGACCCGAAGCTAAAAGTGGATTCAGGAGTACCCTGGAGAAAGCTTTAAACAAGTAAGCACAACAGCCCAAAAAGGACTTTCCGCTAGACCCACTCGAGGAAAACTAAAACCTTGTGAGAGATGAAAGGNCAAAGACGTGGGGGAGGGGGCCTTAACCATGAGGACCAGGTGTGTGTGTGGGGTGGGC

SEQ ID NO: 22 - protein, 75 aa, Human
ANTGAAGGGCCAAAGACGTGGGGGAGGGGGCCTTAACCCATTGAGGACCAGNTGTGTGTGGGGGTGGGGGTGGCC

SEQ ID NO: 23 - protein, 462 aa, Human
  MISC_FEATURE (2)..(2): X = to any amino acid
GXXGAGTGTGGGACGGGGGXGXAATTAAGSYMGGGTAYSGAGCWCGRRKTSCGTTGGTGTMGMCRTTGXXTGKMGAAXGACAGATGSCCATGCCGATTCTTCGAAAGCCATGTTGCMAGAGCCAACGTCAAGCATCHCAAAATTCTCAACACTCCMAACTGTGCCCTTCAGATTGTAGCCCGGCTGAAGAACAACAACAAGACAAGTGTGTGCATTGACCCGAAGCTAAAAGTGGATTCAGGAGTACCTGGAGKAAAGCTTTAAACAAGTAAGCACAACAGCCCAAAAAGGACTTTCCGCTAGACCCACTCGAGGAAAACTAAAACCTTGTGAGAGATGAAAGGSCAAWGACGTKGKGGAGGGGGSCTTAACCCATTGAGGACCAGGTGTGTGTGGGGGTGGCACATTGXATCTTGGGATCGGGCCTGAGGTTTGSCAGCATTTAGACCCTGSATTTATRGC